SEQUENCE LISTING (151 sequences)

SEQ ID NO: 1 - DNA, 1312 bp, Bacillus
  misc_feature: Bacillus sp. BT1B_CT2
gagggaaaaggagcaatccgctttgagatggcctcgcgtccgattagctagttggtgaggtaatggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacaggcatagaaatatgttttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggtagccgtaggagaacctgcggctggatcacctcctttct

SEQ ID NO: 2 - DNA, 1523 bp, Bacillus
  misc_feature: Bacillus sp. BT1B_CT2
agagtttgatcctggctcaggatgaacgctggcggcgtgcttaacacatgcaagtcgaacggagctttgaggaagtgcttgcacagaattaaagcttagtggcggacgggtgagtaacgcgtgaggaacctgcctttcacagtggaataacaacgggaaaccgttgctaatggcgcatgacatcgcattgaggcatctcagagcgattaaaggagaaatccggtgaaagatggactcgcgtccgattagctagttggtgaggtaacggcccaccaaggcgacgatcggtagccgaactgagaggttgatcggccacattgggactgagacacggcccagactcctacgggaggcagcagtggggaatattgggcaatgggcgaaagcctgacccagcaacgccgcgtgaagtatgaaggccttcgggttgtaaacttctttgatcagggaagaaacaaatgacggtacctgaaaaacaagccacggctaactacgtgccagcagccgcggtaatacgtaggtggcgagcgttatccggatttactgggtgtaaagggcgtgcaggcgggctgataagtcagatgtgaaatccccgagcttaactcgggaactgcatctgatactgttggtcttgagtgctggagaggatagtggaattcctagtgtagcggtaaaatgcgcagatattaggaggaacaccagtggcgaaggcggctatctggacagtaactgacgctgaggcgcgaaagcgtgggtagcaaacaggattagataccctggtagtccacgccgtaaacgatgattactaggtgtaggaggtatcgaccccttctgtgccggagttaacacaataagtaatccacctggggagtacggccgcaaggttgaaactcaaaggaattgacgggggcccgcacaagcagtggagtatgtggtttaattcgacgcaacgcgaagaaccttaccagggtttgacatcccttgaacgatgtagagatacataattcccttcggggacaaggagacaggtggtgcatggttgtcgtcagctcgtgtcgtgagatgttgggttaagtcccgcaacgagcgcaacccctattgccagttgccatcatttagttgggcactcaggcgagactgccgttgataaaacggaggaaggtggggatgacgtcaaatcatcatgccccttatatcctgggctacacacgtactacaatggctacaacagagagcagcgacgtcgcgaggcgaagcaaatccccaaatgtagtctcagttcggattgcaggctgcaactcgcctgcatgaagccggaattgctagtaatggcaggtcagcatactgccgtgaatacgttctcgggccttgtacacaccgcccgtcacaccatgagagtcagtaacacccgaagtcagtagtctaaccgcaagggggacgctgccgaaggtgggaccgataattggggtgaagtcgtaacaaggtagccgtatcggaaggtgcggctggatcacctcctttct

SEQ ID NO: 3 - DNA, 1523 bp, Bacillus; identical to SEQ ID NO: 2
  misc_feature: Bacillus sp. BT1B_CT2

SEQ ID NO: 4 - DNA, 1532 bp, Bacillus
  misc_feature: Bacillus sp. BT1B_CT2
tttgatttgagagtttgatcctggctcaggatgaacgctggcggcgtgcttaacacatgcaagtcgaacggagctttgaggaagtgcttgcacagaattaaagcttagtggcggacgggtgagtaacgcgtgaggaacctgcctttcacagtggaataacaacgggaaaccgttgctaatggcgcatgacatcgcattgaggcatctcagagcgattaaaggagaaatccggtgaaagatggactcgcgtccgattagctagttggtgaggtaacggcccaccaaggcgacgatcggtagccgaactgagaggttgatcggccacattgggactgagacacggcccagactcctacgggaggcagcagtggggaatattgggcaatgggcgaaagcctgacccagcaacgccgcgtgaagtatgaaggccttcgggttgtaaacttctttgatcagggaagaaacaaatgacggtacctgaaaaacaagccacggctaactacgtgccagcagccgcggtaatacgtaggtggcgagcgttatccggatttactgggtgtaaagggcgtgcaggcgggctgataagtcagatgtgaaatccccgagcttaactcgggaactgcatctgatactgttggtcttgagtgctggagaggatagtggaattcctagtgtagcggtaaaatgcgcagatattaggaggaacaccagtggcgaaggcggctatctggacagtaactgacgctgaggcgcgaaagcgtgggtagcaaacaggattagataccctggtagtccacgccgtaaacgatgattactaggtgtaggaggtatcgaccccttctgtgccggagttaacacaataagtaatccacctggggagtacggccgcaaggttgaaactcaaaggaattgacgggggcccgcacaagcagtggagtatgtggtttaattcgacgcaacgcgaagaaccttaccagggtttgacatcccttgaacgatgtagagatacataattcccttcggggacaaggagacaggtggtgcatggttgtcgtcagctcgtgtcgtgagatgttgggttaagtcccgcaacgagcgcaacccctattgccagttgccatcatttagttgggcactcaggcgagactgccgttgataaaacggaggaaggtggggatgacgtcaaatcatcatgccccttatatcctgggctacacacgtactacaatggctacaacagagagcagcgacgtcgcgaggcgaagcaaatccccaaatgtagtctcagttcggattgcaggctgcaactcgcctgcatgaagccggaattgctagtaatggcaggtcagcatactgccgtgaatacgttctcgggccttgtacacaccgcccgtcacaccatgagagtcagtaacacccgaagtcagtagtctaaccgcaagggggacgctgccgaaggtgggaccgataattggggtgaagtcgtaacaaggtagccgtatcggaaggtgcggctggatcacctcctttct

SEQ ID NO: 5 - DNA, 1312 bp, Bacillus; identical to SEQ ID NO: 1
  misc_feature: Bacillus sp. BT1B_CT2

SEQ ID NO: 6 - DNA, 1533 bp, Bacillus
  misc_feature: Bacillus sp. BT1B_CT2
tacaatgaagagtttgatcctggctcaggatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatgggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccctagggtaaaactggtgactggggctaagtcgtaacaaggtagccgtaccggaaggtgcggctggaacacctcctt

SEQ ID NO: 7 - DNA, 1531 bp, Bacillus
  misc_feature: Bacillus sp. BT1B_CT2
ttacaatgaagagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcataattcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcctgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacctcctttct

SEQ ID NO: 8 - DNA, 1567 bp, Bacillus
  misc_feature: Bacillus sp. BT1B_CT2
tggagagtttgatcctggctcaggacgaacgctggcggcgtgcttaacacatgcaagtcgaacgagaggacatgggaagcttgcttcctatgaaatcgagtggcaaacgggtgagtaacgcgtaaacaacctgcccttcggatggggataacagccggaaacggctgctaataccgaatacgatcttttcgtcgcatgacggaaagaagaaaggatggcctctatacaaagctatcgccgaaggaggggtttgcgtctgattagctggttggaggggtaacggcccaacaaggcgatgatcagtagccggtctgagaggatgaacggccacattgggactgagacacggcccagactcctacgggaggcagcagtggggaatcttccgcaatggacgaaagtctgacggagcaacgccgcgtgagtgaagaaggtcttcggattgtaaaagctctgttatacgggacgaaaaagacggatgccaacagtatccgtccgtgacggtaccgtaagagaaagccacggctaactacgtgccagcagccgcggtaatacgtaggtggcaagcgttgtccggaattattgggcgtaaagggcgcgcaggcggttcggtaagtctgtcttaaaagtgcggggcttaaccccgtgaggggacggaaactgtcgaacttgagtgtcggagaggaaagcggaattcctagtgtagcggtgaaatgcgtagatattaggaggaacaccggtggcgaaagcggctttctggacgacaactgacgctgaggcgcgaaagcgtggggagcaaacaggattagataccctggtagtccacgccgtaaacgatggatactaggtgtaggaggtatcgaccccttctgtgccgtagttaacgctataagtatcccgcctggggagtacggccgcaaggttgaaactcaaaggaattgacgggggcccgcacaagcggtggagtatgtggtttaattcgacgcaacgcgaagaaccttaccaagccttgacattgatcgcaattttcagagatgagaagttcctcttcggaggacgagaaaacaggtggtgcacggctgtcgtcagctcgtgtcgtgagatgttgggttaagtcccgcaacgagcgcaacccctatcttctgttaccagcacgtaagggtggggactcaggagagactgccgcagacaatgcggaggaaggcggggatgacgtcaagtcatcatgccccttatggcttgggctacacacgtactacaatggctctaaatagagggaagcgaaggagcgatccggagcaaaacccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccgcaaggagccagccgtcgaaggtgggggcgatgattggggtgaagtcgtaacaaggtagccgtatcggaaggtgcggctggatcacctcctttct

SEQ ID NO: 9 - DNA, 1543 bp, Bacillus
  misc_feature: Bacillus sp. BT1B_CT2
atcaaatggagagtttgatcctggctcaggatgaacgctggcggcatgcctaatacatgcaagtcgaacgaagtctctaggagcttgcttccaaagagacttagtggcgaacgggtgagtaacacgtaggtaacctgcccatgtgtctgggataactgctggaaacggtagctaaaaccggataggtatacggagcgcatgctctgtatattaaagcacccctcaaggtgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatggggggaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtaggctagagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggatgcaaaggctctagagatagagagataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacttggtgacaaggagcgaatctcataaaggccgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccgatgactggggttaagtcgtaacaaggtatccctacgggaacgtggggatggatcacctcctttct

SEQ ID NO: 10 - DNA, 1554 bp, Bacillus
  misc_feature: Bacillus sp. BT1B_CT2
acttcatggagagtttgatcctggctcaggacgaacgctggcggcgtgcctaatacatgcaagtcgagcggaccgacgggagcttgctcccttaggtcagcggcggacgggtgagtaacacgtgggtaacctgcctgtaagactgggataactccgggaaaccggggctaataccggatgcttgattgaaccgcatggttcaatcataaaaggtggcttttagctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgttagggaagaacaagtaccgttcgaatagggcggtaccttgacggtacctaaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagcaaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggcttccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatgggcagaacaaagggcagcgaagccgcgaggctaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaaccttttggagccagccgccgaaggtgggacagatgattggggtgaagtcgtaacaaggtagccgtatcggaaggtgcggctggatcacctcctttct

SEQ ID NO: 11 - DNA, 1543 bp, Bacillus; identical to SEQ ID NO: 9
  misc_feature: Bacillus sp. BT1B_CT2

SEQ ID NO: 12 - DNA, 1554 bp, Bacillus; identical to SEQ ID NO: 10
  misc_feature: Bacillus sp. BT1B_CT2

SEQ ID NO: 13 - DNA, 1488 bp, Bacteroides coprocola
  misc_feature: Bacteroides coprocola M16
agagtttgatcctggctcaggatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatggcaccattaaagatttattggtaaacgatggggatgcgttccattaggtagtaggcggggtaacggcccacctagcctgcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgagggctctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaaccggtaattggggctaagtctaacaaggtaacc

SEQ ID NO: 14 - DNA, 1518 bp, Bacteroides coprocola
  misc_feature: Bacteroides coprocola M16
gtttgatcctggctcaggatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatggcaccattaaagatttattggtaaacgatggggatgcgttccattaggtagtaggcggggtaacggcccacctagcctgcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggttataaacttcttttatacgggaataaagttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgagggctctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcacggccatgaaagccgggggtacctgaagtgcgtaaccgcaagagcgccctagggtaaaaccggtaattggggctaagtcgtaacaaggtagccgtaccggaaggtgcggctggaacacctcctttct

SEQ ID NO: 15 - DNA, 1532 bp, Bacteroides coprocola
  misc_feature: Bacteroides coprocola M16
ttacaatgaagagtttgatcctggctcaggatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatggcaccattaaagatttattggtaaacgatggggatgcgttccattaggtagtaggcggggtaacggcccacctagcctgcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgagggctctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaaccggtaattggggctaagtcgtaacaaggtagccgtaccggaaggtgcggctggaacacctcctttct

SEQ ID NO: 16 - DNA, 1532 bp, Bacteroides coprocola; identical to SEQ ID NO: 15
  misc_feature: Bacteroides coprocola M16

SEQ ID NO: 17 - DNA, 1532 bp, Bacteroides coprocola; identical to SEQ ID NO: 15
  misc_feature: Bacteroides coprocola M16

SEQ ID NO: 18 - DNA, 1532 bp, Bacteroides coprocola; identical to SEQ ID NO: 15
  misc_feature: Bacteroides coprocola M16

SEQ ID NO: 19 - DNA, 1532 bp, Bacteroides coprocola; identical to SEQ ID NO: 15
  misc_feature: Bacteroides coprocola M16

SEQ ID NO: 20 - DNA, 1532 bp, Bacteroides coprocola; identical to SEQ ID NO: 15
  misc_feature: Bacteroides coprocola M16

SEQ ID NO: 21 - DNA, 1539 bp, Bacteroides coprocola
  misc_feature: Bacteroides coprocola M16
ttttacaatgaagagtttgatcctggctcaggatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatggcaccattaaagatttattggtaaacgatggggatgcgttccattaggtagtaggcggggtaacggcccacctagcctgcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgagggctctggccagactgccatcgtaagatgtgaggaaggtggggatgacgtcaaatcagcacggcccttacgtccggggctacacacgtgttacaatgggaggtacagaaggccgctacccggcaacgggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaaccggtaattggggctaagtcgtaacaaggtagccgtaccggaaggtgcggctggaacacctcctttctggagc

SEQ ID NO: 22 - DNA, 1539 bp, Bacteroides coprocola; identical to SEQ ID NO: 21
  misc_feature: Bacteroides coprocola M16

SEQ ID NO: 23 - DNA, 1539 bp, Bacteroides coprocola; identical to SEQ ID NO: 21
  misc_feature: Bacteroides coprocola M16

SEQ ID NO: 24 - DNA, 1274 bp, Bacteroides coprocola
  misc_feature: Bacteroides coprocola M16
ttttacaatgaagagtttgatcctggctcaggatgaacgctagctacaggcttaacacatgcaagtcgaggggcagcatgaacttagcttgctaagtttgatggcgaccggcgcacgggtgagtaacacgtatccaaccttccgtttactcagggatagcctttcgaaagaaagattaatacctgatagtatggtgagattgcatgatggcaccattaaagatttattggtaaacgatggggatgcgttccattaggtagtaggcggggtaacggcccacctagcctgcgatggataggggttctgagaggaaggtcccccacattggaactgagacacggtccaaactcctacgggaggcagcagtgaggaatattggtcaatgggcgagagcctgaaccagccaagtagcgtgaaggatgaaggtcctacggattgtaaacttcttttatacgggaataaagtttcctacgtgtaggattttgtatgtaccgtatgaataagcatcggctaactccgtgccagcagccgcggtaatacggaggatgcgagcgttatccggatttattgggtttaaagggagcgcagacgggagattaagtcagttgtgaaagtttgcggctcaaccgtaaaattgcagttgatactggtttccttgagtgcagttgaggcaggcggaattcgtggtgtagcggtgaaatgcttagatatcacgaagaaccccgattgcgaaggcagcttgctaaactgtaactgacgttcatgctcgaaagtgtgggtatcaaacaggattagataccctggtagtccacacggtaaacgatggatactcgctgttggcgatatactgtcagcggccaagcgaaagcattaagtatcccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaaccctcgtggtcagttactaacaggttaagctgagggctctggccagactgccatcgtagatgtgaggaaggtggggatgacgtcaaatcagcacgcctacgtcgggctaccacgtgtacatgggagtacagaaggccgctaccggcacggatgccaatccccaaa

SEQ ID NO: 25 - DNA, 1516 bp, Bacteroides eggerthii
  misc_feature: Bacteroides eggerthii CCUG 9559
gtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggccaatggacgagagtctgaaccagccaagtagcgtgaaggatgactgccctatggttttaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaccgcaggagcgtcctagggtaaaactggtgattggggctaagtcgtaacaaggtagccgtaccggaaggtgcggctggaacacctcctttct

SEQ ID NO: 26 - DNA, 1539 bp, Bacteroides eggerthii
  misc_feature: Bacteroides eggerthii CCUG 9559
cttttacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctttctggagc

SEQ ID NO: 27 - DNA, 1527 bp, Bacteroides eggerthii
  misc_feature: Bacteroides eggerthii CCUG 9559
tacaatgaagagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtagccgtaccggaaggtgcggctggaacacctcctt

SEQ ID NO: 28 - DNA, 1527 bp, Bacteroides eggerthii; identical to SEQ ID NO: 27
  misc_feature: Bacteroides eggerthii CCUG 9559

SEQ ID NO: 29 - DNA, 1421 bp, Bacteroides eggerthii
  misc_feature: Bacteroides eggerthii CCUG 9559
ttgatcatggctcaggatgaacgttagctacaggacttacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcattattaaagaatttcggttatcgatggggatgcgttccattagatagttggcggggtaacggcccaccaagtcaacgatggataggggttctgagaggaaggtcccccacattggaactgagacacggtccaaattcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcggccgctgtgaagtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgmtagtaatcgcgcatcagccacggcgcggtgaatacgttcccgggccttgtacacaccgcccgtcaagccatgaaagccgggggtacctgaagtacgta

SEQ ID NO: 30 - DNA, 1488 bp, Bacteroides eggerthii
  misc_feature: Bacteroides eggerthii CCUG 9559
agagtttgatcctggctcaggatgaacgctagctacaggcttaacacatgcaagtcgaggggcagcatgattgaagcttgcttcaatcgatggcgaccggcgcacgggtgagtaacacgtatccaacctgccgataactcggggatagcctttcgaaagaaagattaatacccgatagtatagtttttccgcatggtttcactattaaagaatttcggttatcgatggggatgcgttccattagatagttggcggggtaacggcccaccaagtcaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtggagtatgcatactcctttgtatgtaccgtatgaataaggatcggctaactccgtgccagcagccgcggtaatacggaggatccgagcgttatccggatttattgggtttaaagggagcgtaggcgggtgcttaagtcagttgtgaaagtttgcggctcaaccgtaaaattgcagttgatactgggcgccttgagtgcagcataggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgttggcgatacacagtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagcggaatgtagtggaaacattacagccttcgggccgctgtgaaggtgctgcatggttgtcgtcagctcgtgccgtgaggtgtcggcttaagtgccataacgagcgcaacccttatctatagttactatcaggtcatgctgaggactctatggagactgccgtcgtaagatgtgaggaaggtggggatgacgtcaaatcagcacggcccttacgtccggggctacacacgtgttacaatggggggtacagaaggcagctacctggcgacaggatgctaatcccgaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaaggagcgtcctagggtaaaactggtgattggggctaagtcgtaacaaggtaacc

SEQ ID NO: 31 - DNA, 1531 bp, Bacteroides vulgatus
  misc_feature: Bacteroides vulgatus 8482
  misc_feature (33)..(33): n is a, c, g, or t
  misc_feature (95)..(95): n is a, c, g, or t
  misc_feature (97)..(97): n is a, c, g, or t
  misc_feature (393)..(393): n is a, c, g, or t
  misc_feature (479)..(479): n is a, c, g, or t
  misc_feature (542)..(542): n is a, c, g, or t
  misc_feature (742)..(742): n is a, c, g, or t
  misc_feature (930)..(930): n is a, c, g, or t
  misc_feature (1202)..(1202): n is a, c, g, or t
  misc_feature (1253)..(1253): n is a, c, g, or t
  misc_feature (1497)..(1497): n is a, c, g, or t
  misc_feature (1508)..(1516): n is a, c, g, or t
ttacaatgaagagtttgatcctggctcaggatnaacgctagctacaggcttaacacatgcaagtcgaggggcagcatggtcttagcttgctaagncnatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagccngaaccagccaagtagcgtgaaggatgactgccctatgggttgtaaacttcttttataaaggaataaagtcgggtatggatacccgnttgcatgtactttatgaataaggatcggctaactccgtgccagcagccgcggtaatacggagnatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctnagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggccngcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttatgctgaggactctgacaagactgccatcgtaagatgtgaggaaggtggggatgacgtcaaatcagcacngcccttacgtccggggctacacacgtgttacaatggggggtacagagggcngctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagtcatgggagccgggggtacctgaagtgcgtaaccgcgaggagcgccctagggtaaaactggtgactggggctaagtcgtaacaaggtagcngtaccggaagnnnnnnnnngaacacctcctttct

SEQ ID NO: 32 - DNA, 1393 bp, Bacteroides vulgatus
  misc_feature: Bacteroides vulgatus 8482
caggcttaccacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaagatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaacttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgta

SEQ ID NO: 33 - DNA, 1520 bp, Bacteroides vulgatus
  misc_feature: Bacteroides vulgatus 8482
gtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttct

SEQ ID NO: 34 - DNA, 1532 bp, Bacteroides vulgatus
  misc_feature: Bacteroides vulgatus 8482
ttacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatatttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttttgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttct

SEQ ID NO: 35 - DNA, 1532 bp, Bacteroides vulgatus; identical to SEQ ID NO: 34
  misc_feature: Bacteroides vulgatus 8482

SEQ ID NO: 36 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 37 - DNA, 1538 bp, Bacteroides vulgatus; identical to SEQ ID NO: 36
  misc_feature: Bacteroides vulgatus 8482

SEQ ID NO: 38 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 39 - DNA, 1538 bp, Bacteroides vulgatus
  misc_feature: Bacteroides vulgatus 8482
tattacaatgaagagtttgatcctagctcaggatgaacgctagctacaggcttaacacatgcaagtcgaggggcagcatggtcttagcttgctaaggccgatggcgaccggcgcacgggtgagtaacacgtatccaacctgccgtctactcttggatagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtcatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 40 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaggaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttctgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 41 - DNA, 1538 bp, Bacteroides vulgatus
  misc_feature: Bacteroides vulgatus 8482
tattacaatgaagagtttgatcctggctcaggatgaacgctagctacaggcttaacacatgcaagtcgaggggcagcatggtcttagcttgctaagactgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggtttcgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaaggtgcggctggaacacctcctttctggag

SEQ ID NO: 42 - DNA, 1489 bp, Bacteroides vulgatus
  misc_feature: Bacteroides vulgatus 8482
agagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtaacc

SEQ ID NO: 43 - DNA, 1486 bp, Butyricimonas virosa
  misc_feature: Butyricimonas virosa MT12
agagtttgatcctggctcaggatgaacgctagcgacaggcttaacacatgcaagtcgaggggcagcacggtgtagcaatacactggtggcgaccggcgcacgggtgagtaacacgtgtgcaaccaaccccgtaccgggagataacccgcggaaacgtggactaacatcccatgatactcgagagccgcatggctctcgatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgagagcctgaaccagccaagtcgcgtgagggaagaatggtctatggcctgtaaacctcttttgtcagggaagaataaggatgacgagtcattcgatgccagtacttgacgaataagcatcggctaactccgtgccagcagccgcggtaatacgggggatgcgagcgttatccggatttattgggtttaaagggcgcgtaggcgggacgtcaagtcagcggtaaaagactgcagctaaactgtagcacgccgttgaaactggcgccctggagacgagacgagggaggcggaacaagtgaagtagcggtgaaatgcatagatatcacttggaaccccgatagcgaaggcagcttcccaggctcgttctgacgctgatgcgcgagagcgtgggtagcgaacaggattagataccctggtagtccacgccgtaaacgatgctcactggatcttggcgatacactgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtagattgcatgaggtggaaacgcttcttcccttcggggctatttacaaggtgctgcatggttgtcgtcagctcgtgccgtgaggtgtcgggttaagtcccataacgagcgcaacccctatcgccagttgccatcggttgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcgaggaacgggctagggtaataccggtaactggggctaagtcgtaacaaggtaacc

SEQ ID NO: 44 - DNA, 1440 bp, Coprococcus eutactus
  misc_feature: Coprococcus eutactus ATCC 27759
gatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacrgtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcraacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtg

SEQ ID NO: 45 - DNA, 1298 bp, Coprococcus eutactus
  misc_feature: Coprococcus eutactus ATCC 27759
gatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttctaaggaa

SEQ ID NO: 46 - DNA, 1528 bp, Coprococcus eutactus
  misc_feature: Coprococcus eutactus ATCC 27759
tttttaatgagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacagtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcgaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttctaaggaa

SEQ ID NO: 47 - DNA, 1493 bp, Coprococcus eutactus
  misc_feature: Coprococcus eutactus ATCC 27759
cgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggtttcttaaccgagacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttctaaggaa

SEQ ID NO: 48 - DNA, 1528 bp, Coprococcus eutactus
  misc_feature: Coprococcus eutactus ATCC 27759
tttttaatgagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacagtttcttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttctaaggaa

SEQ ID NO: 49 - DNA, 1487 bp, Coprococcus eutactus
  misc_feature: Coprococcus eutactus ATCC 27759
cgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggtttcttaaccgagacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 50 - DNA, 1292 bp, Coprococcus eutactus
  misc_feature: Coprococcus eutactus ATCC 27759
gatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 51 - DNA, 1514 bp, Coprococcus eutactus
  misc_feature: Coprococcus eutactus ATCC 27759
gagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacagtttcttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 52 - DNA, 1514 bp, Coprococcus eutactus
  misc_feature: Coprococcus eutactus ATCC 27759
gagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacagtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcgaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 53 - DNA, 1522 bp, Coprococcus eutactus
  misc_feature: Coprococcus eutactus ATCC 27759
tttttaatgagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacggtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacagtttcttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcaaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 54 - DNA, 1522 bp, Coprococcus eutactus
  misc_feature: Coprococcus eutactus ATCC 27759
tttttaatgagagtttgatcctggctcaggatgaacgctggcggcgtgcttaacacatgcaagtcgaacgattgaagctggagcttgctccggccgatttagtggcggacgggtgagtaacgcgtgggtaacctgcctcatacagggggatagcagttggaaacgactggtaaaaccgcataagcgcacagtatcgcatgatacagtgtgaaaacactccggtggtatgagatggacccgcgtctgattagctagttggtgaggtaacggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggtgcgtaggtggcaaggcaagtctgaagtgaaaatccggggctcaaccccggaactgctttggaaactgtttagctagagtacaggagaggtaagtggaattcctagtgtagcggtgaaatgcgtagatattaggaggaacaccagtggcgaaggcgacttactggactgctactgacactgaggcacgaaagcgtggggagcaaacaggattagataccctggtagtccacgccgtaaacgatgaatactaggtgttgggttccaaagggactcggtgccgtcgcaaacgcattaagtattccacctggggagtacgttcgcaagaatgaaactcaaaggaattgacggggacccgcacaagcggtggagcatgtggtttaattcgaagcaacgcgaaaaaccttaccaagtcttgacatcctgatgacggttccttaaccggaacttctcttcggagcatcagagacaggtggtgcatggttgtcgtcagctcgtgtcgtgagatgttgggttaagtcccgcaacgagcgcaacccctatttccagtagccagcagtaagatgggcactctggagagactgccggggataacccggaggaaggtggggatgacgtcaaatcatcatgccccttatgatttgggctacacacgtgctacaatggcagttacaaagagaagcgaacctgtgaaggcgagcaaacctcaaaaaggctgtctcagttcggattgtagtctgcaactcgactacatgaagctggaatcgctagtaatcgcagatcagcatgctgcggtgaatacgttcccgggtcttgtacacaccgcccgtcacaccatgggagtcggtaacgcccgaagtcagtgacccaaccgcaaggagggagctgccgaaggcaggatcgataactggggtgaagtcgtaacaaggtagccgtatcggaaggtgcggctggatcacctcctttct

SEQ ID NO: 55 - DNA, 1292 bp, Coprococcus eutactus; identical to SEQ ID NO: 50
  misc_feature: Coprococcus eutactus ATCC 27759

SEQ ID NO: 56 - DNA, 1487 bp, Coprococcus eutactus; identical to SEQ ID NO: 49
  misc_feature: Coprococcus eutactus ATCC 27759

SEQ ID NO: 57 - DNA, 1542 bp, Desulfovibrio piger
  misc_feature: Desulfovibrio piger DSM 749
agagtttgatcctggctcagattgaacgctggcggcgtgcttaacacatgcaagtcgtacgcgaaagggacttcggtcccgagtaaagtggcgcacgggtgagtaacacgtggataatctgcctctatgatggggataacagttggaaacgactgctaataccgaatacgctcatgatgaactttgtgaggaaaggtggcctctgcttgcaagctatcgcatagagatgagtccgcgtcccattagctagttggtggggtaacggcctaccaaggcaacgatgggtagccgatctgagaggatgatcggccacactggaactgaaacacggtccagactcctacgggaggcagcagtggggaatattgcgcaatgggcgaaagcctgacgcagcgacgccgcgtgagggatgaaggtcttcggatcgtaaacctctgtcagaagggaagaaactagggtgttctaatcatcatcctactgacggtaccttcaaaggaagcaccggctaactccgtgccagcagccgcggtaatacggagggtgcaagcgttaatcggaatcactgggcgtaaagcgcacgtaggctgttatgtaagtcaggggtgaaagcccacggctcaaccgtggaactgcccttgatactgcacgactcgaatccgggagagggtggcggaattccaggtgtaggagtgaaatccgtagatatctggaggaacatcagtggcgaaggcggccacctggaccggtattgacgctgaggtgcgaaagcgtggggagcaaacaggattagataccctggtagtccacgccgtaaacgatggatgctagatgtcgggatgtatgtctcggtgtcgtagttaacgcgttaagcatcccgcctggggagtacggtcgcaaggctgaaactcaaagaaattgacgggggcccgcacaagcggtggagtatgtggtttaattcgatgcaacgcgaagaaccttacctaggtttgacatctggggaaccctcccgaaaatgaggggtgcccttcggggagccccaagacaggtgctgcatggctgtcgtcagctcgtgtcgtgagatgttgggttaagtcccgcaacgagcgcaacccctatgcatagttgccagcaagtaaagttgggcactctatgcagactgcccgggttaaccgggaggaaggtggggacgacgtcaagtcatcatggcccttacacctagggctacacacgtactacaatggcacgcacaaagggcagcgataccgtgaggtggagccaatcccaaaaaacgtgtcccagtccggattgcagtctgcaactcgactgcatgaagtcggaatcgctagtaattcgaggtcagcatactcgggtgaatgcgttcccgggccttgtacacaccgcccgtcacaccacgaaagtcggttttacccgaagccggtgagccaactagcaatagaggcagccgtctacggtagggccgatgattggggtgaagtcgtaacaaggtagccgtaggggaacctgcggctggatcacctcctt

SEQ ID NO: 58 - DNA, 1523 bp, Dolosigranulum pigrum
  misc_feature: Dolosigranulum pigrum IFO 15550
  misc_feature (225)..(225): n is a, c, g, or t
tttgatcctggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacgaattcattaaaggtggctttgctaccactcatggatantcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcgtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgccgtaaacgatgagtgctaagtgttgggggctttccggccctcggtgctggagctaacgtattaagcactccgcctggggattacgaccgcaaggttgaaactcaaaggaattgacggggaccgcacaagcggtggagcatgtggtttaattcgaagcaacgcgaagaacttaccagctcttgacatcttctgacaactctagagatagagcgttcttcggggacagaatgacaggtggtgcatggttgtcgtcagctcgtgtcgtgagatgttgggttaagtcccgtaagcagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacc

SEQ ID NO: 59 - DNA, 1551 bp, Dolosigranulum pigrum
  misc_feature: Dolosigranulum pigrum IFO 15550
ttaaacatgagagtttgatcttggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacgaattcattaaaggtggctctgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgccgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttct

SEQ ID NO: 60 - DNA, 1331 bp, Dolosigranulum pigrum
  misc_feature: Dolosigranulum pigrum IFO 15550
gctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgctgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttct

SEQ ID NO: 61 - DNA, 1551 bp, Dolosigranulum pigrum
  misc_feature: Dolosigranulum pigrum IFO 15550
ttaaacatgagagtttgatcttggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgactaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgctgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttct

SEQ ID NO: 62 - DNA, 1472 bp, Dolosigranulum pigrum
  misc_feature: Dolosigranulum pigrum IFO 15550
  misc_feature (171)..(171): n is a, c, g, or t
  misc_feature (788)..(788): n is a, c, g, or t
gacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacnaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgcngtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaag

SEQ ID NO: 63 - DNA, 1551 bp, Dolosigranulum pigrum; identical to SEQ ID NO: 61
  misc_feature: Dolosigranulum pigrum IFO 15550

SEQ ID NO: 64 - DNA, 1551 bp, Dolosigranulum pigrum; identical to SEQ ID NO: 59
  misc_feature: Dolosigranulum pigrum IFO 15550

SEQ ID NO: 65 - DNA, 1331 bp, Dolosigranulum pigrum; identical to SEQ ID NO: 60
  misc_feature: Dolosigranulum pigrum IFO 15550

SEQ ID NO: 66 - DNA, 1557 bp, Dolosigranulum pigrum
  misc_feature: Dolosigranulum pigrum IFO 15550
tttaaacatgagagtttgatcttggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgactaattcattaaaggtggctttgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgctgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttctaagga

SEQ ID NO: 67 - DNA, 1557 bp, Dolosigranulum pigrum
  misc_feature: Dolosigranulum pigrum IFO 15550
tttaaacatgagagtttgatcttggctcaggacgaacgctggcggcatgcctaatacatgcaagtcgaacgatgatatcactgcttgcagtgattgattagtggcgaacgggtgagtaacacgtgaggaacttgcccatgagcgggggacaacattcggaaacggatgctaataccccataggtggattggtcgcatgacgaattcattaaaggtggctctgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgccgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttctaagga

SEQ ID NO: 68 - DNA, 1337 bp, Dolosigranulum pigrum
  misc_feature: Dolosigranulum pigrum IFO 15550
tgctaccactcatggatagcctcgcggcgtattagctagttggtaaggtaatggcttaccaaggcagtgatacgtagccgacttgagagggtgatcggccacactgggactgagacacggcccagactcctacgggaggcagcagtagggaatcttccacaatgggtgcaaacctgatggagcaatgccgcgtgagtgaagaaggtcttcggatcgtaaagctctgttgttagagaagaacacgtgctaggtaactactagcgccttgacggtatctaaccagaaagtcacggctaactacgtgccagcagccgcggtaatacgtaggtgacaagcgttgtccggatttattgggcgtaaagggagcgcaggcggtctgtttagtctaatgtgaaagcccacggcttaaccgtggaacggcattggaaactgacagacttgaatgtagaagaggaaaatggaattccaagtgtagcggtggaatgcgtagatatttggaggaacaccagtggcgaaggcgattttctggtctaacattgacgctgaggctcgaaagcgtggggagcgaacaggattagataccctggtagtccacgctgtaaacgatgagtgctaagtgttgggggctttccggccctcggtgctggagctaacgtattaagcactccgcctggggattacgaccgcaaggttgaaactcaaaagaattgacggggacccgcacaagcggtggagcatgtggtttaattcgaagcaacgcgaagaaccttaccagctcttgacatcttctgacaactctagagatagagcgttcccttcggggacagaatgacaggtggtgcatggttgtcgtcagctcgtgtcgtgagatgttgggttaagtcccgtaacgagcgcaacccttgttgttagttgccagcattaagttgggcactttaacgagactgccggtgataaaccggaggaaggcggggatgacgtcaaatcatcatgccccttatgagctgggctacacacgtgctacaatggatggtacaacgagcagcgatcccgcaagggctagctaatctttcaaagccattctcagttcggattgcactctgcaactcgagtgcatgaagtcggaatcgctagtaatcgcgaatcagaatgtcgcggtgaatccgttcccgggtcttgtacacaccgcccgtcacaccacgagagtttgaaacacccgaagtcggccggccaaccctatgggaggcagccgtcgaaggtgggttgaatgattggggtgaagtcgtaacaaggtagccgtatcggaaggtgcggctggatcacctcctttctaagga

SEQ ID NO: 69 - DNA, 1483 bp, Eubacterium biforme
  misc_feature: Eubacterium biforme DSM 3989
  misc_feature (27)..(27): n is a, c, g, or t
  misc_feature (116)..(116): n is a, c, g, or t
  misc_feature (238)..(238): n is a, c, g, or t
  misc_feature (337)..(338): n is a, c, g, or t
  misc_feature (575)..(575): n is a, c, g, or t
  misc_feature (924)..(925): n is a, c, g, or t
  misc_feature (929)..(929): n is a, c, g, or t
  misc_feature (964)..(964): n is a, c, g, or t
  misc_feature (1225)..(1225): n is a, c, g, or t
  misc_feature (1381)..(1381): n is a, c, g, or t
tggagagtttgatcctggctcaggatnaacgctggcggcatgcctaatacatgcaagtcgaacgagaggaaggaaagcttgcttttctgaatctagtggcgaacgggtgagtaacncgtaggtaacctgcccatgtgcccgggataacttctggaaacggatgctaaaaccggataggtagcagacaagcatttgactgctattaaagtggctaaggccatgaacatggatggacctncggtgcattagctagttggtgaggtaacggcccaccaaggcgacgatgcatagccggcctgagagggcggacggccacattgggactgagacacggccnnaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtanagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacggnngccngcacaagcggtggagtatgtggtttaattcgaagnaacgcgaagaaccttaccaggccttgacatcccttgcagagrtatagagatatatccgaggttaacaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtnctacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggcnttgtacacaccgcccgtcaaaccatgggagttggtaatgcccgaagccggtggcataacctttcgaggagtgagccgtcgaaggcaggaccgatgactgggg

SEQ ID NO: 70 - DNA, 1548 bp, Eubacterium biforme
  misc_feature: Eubacterium biforme DSM 3989
caaatggagagtttgatcctggctcaggatgaacgctggcggcatgcctaatacatgcaagtcgaacgagaggaaggaaagcttgcttttctgaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtaaagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtggcataacctttcgaggagtgagccgtcgaaggcaggaccgatgactggggttaagtcgtaacaaggtatccctacgggaacgtggggatggatcacctcctttctaaggag

SEQ ID NO: 71 - DNA, 1538 bp, Eubacterium biforme
  misc_feature: Eubacterium biforme DSM 3989
tggagagtttgatcctggctcaggatgaacgctggcggcatgcctaatacatgcaagtcgaacgagaggaaggaaagcttgcttttctgaatctagtggcgaacgggtgagtaacacgtaggtaacctgcccatgtgcccgggataacttctggaaacggatgctaaaaccggataggtagcagacaagcatttgactgctattaaagtggctaaggccatgaacatggatggacctgcggtgcattagctagttggtgaggtaacggcccaccaaggcgacgatgcatagccggcctgagagggcggacggccacattgggactgagacacggcccaaactcctacgggaggcagcagtagggaattttcgtcaatggggggaaccctgaacgagcaatgccgcgtgagtgaggaaggtcttcggatcgtaaagctctgttgtaagagaaaaacgacattcatagggaatgatgagtgagtgatggtatcttaccagaaagtcacggctaactacgtgccagcagccgcggtaatacgtaggtggcgagcgttatccggaatgattgggcgtaaagggtgcgtaggtggcagaacaagtctggagtaaaaggtatgggctcaacccgtactggctctggaaactgttcagctagagaacagaagaggacggcggaactccatgtgtagcggtaaaatgcgtagatatatggaagaacaccggtggcgaaggcggccgtctggtctgttgctgacactgaagcacgaaagcgtggggagcaaataggattagataccctagtagtccacgccgtaaacgatgagaactaggtgttgggggaataactcagtgccgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatcccttgcagaggtatagagatatatccgaggttatcaaggagacaggtggtgcatggttgtcgtcagctcgtgtcgtgagatgttgggttaagtcccgcaacgagcgcaacccttgtgatatgttactaacattgagttgaggactcatatcagactgccggtgataaaccggaggaaggtggggatgacgtcaaatcatcatgccccttatggcctgggctacacacgtactacaatggcgtctacaaagagcggcaagcctgtgaaggcaagcgaatctcataaaggacgtctcagttcggattgaagtctgcaacccgacttcatgaagctggaatcgctagtaatcgcgaatcagcatgtcgcggtgaatacgttctcgggccttgtacacaccgcccgtcaaaccatgggagttggtaatgcccgaagccggtggcataacctttcgaggagtgagccgtcgaaggcaggaccgatgactggggttaagtcgtaacaaggtatccctacgggaacgtggggatggatcacctcctttct

SEQ ID NO: 72 - DNA, 1538 bp, Eubacterium biforme; identical to SEQ ID NO: 71
  misc_feature: Eubacterium biforme DSM 3989

SEQ ID NO: 73 - DNA, 1474 bp, Eubacterium eligens
  misc_feature: Eubacterium eligens DSM 3376
  misc_feature (534)..(534): n is a, c, g, or t
  misc_feature (601)..(601): n is a, c, g, or t
  misc_feature (615)..(617): n is a, c, g, or t
  misc_feature (921)..(921): n is a, c, g, or t
  misc_feature (955)..(956): n is a, c, g, or t
  misc_feature (1183)..(1183): n is a, c, g, or t
  misc_feature (1389)..(1389): n is a, c, g, or t
  misc_feature (1466)..(1466): n is a, c, g, or t
tgagagtttgatcctggctcaggatgaacgctggcggcgtgcttaacacatgcaagtcgaacgaagcatttacgaacagattayttcggtatgaagttcctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcnagcgttatccggatttactgggtgtaaagggagtgtaggtggccatgcaagtcagaagtgaaaatcnggggctyaaccccnnnactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggaccngcacaagcggtggagcatgtggtttaattcgaannaacgcgaagaaccttaccaagtcttgacatcccactgaccggacagtaatgtgtcctttcttcgggacartrgagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcnaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgnccgtcacaccatgggagtcgaaaatgcccgaagtcggtgacctaacgtaagaaggagccgccgaaggcaggtttganaactgggg

SEQ ID NO: 74 - DNA, 1535 bp, Eubacterium eligens
  misc_feature: Eubacterium eligens DSM 3376
ctttttatgagagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtggcctttccttcgggacaagagagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttct

SEQ ID NO: 75 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattacttcggtttgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcctcttgaccggtcagtaatgtggcctttccttcgggacaagagagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 76 - DNA, 1541 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccaaaagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 77 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatagtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggcttaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggtgacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgaaagaaggagccgccgaaggcaggtttgataactggggtgaagtcgtaacaaggtagccgtatcggaaggtgcggctggatcacctcctttctaaggaa

SEQ ID NO: 78 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttagaacagattatttcggtatgaagttctttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtgaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcaaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgcaagaaggagccgccgaaggcaggtttgataactggggtgaagtcgtaacaaggtagccgtatcggaaggtgcggctggatcacctcctttctaaggaa

SEQ ID NO: 79 - DNA, 1540 bp, Eubacterium eligens
  misc_feature: Eubacterium eligens DSM 3376
tttttatgagagtttgatcctggctcaggatgaacgctggcggcgtgcttaacacatgcaagtcgaacgaagcatttgcgacagattttttcggaatgaagttgcttatgactgagtggcggacgggtgagtaacgcgtgggtaacctgccttgtactgggggatagcagctggaaacggctggtaataccgcataagcgcacaatgttgcatgacatggtgtgaaaaactccggtggtataagatggacccgcgtctgattagctagttggtgagataacagcccaccaaggcgacgatcagtagccgacctgagagggtgaccggccacattgggactgagacacggcccagactcctacgggaggcagcagtggggaatattgcacaatggaggaaactctgatgcagcgacgccgcgtgagtgaagaagtaattcgttatgtaaagctctatcagcagggaagatagtgacggtacctgactaagaagctccggctaaatacgtgccagcagccgcggtaatacgtatggagcaagcgttatccggatttactgggtgtaaagggagtgtaggtggccatgcaagtcagaagtgaaaatccggggctcaaccccggaactgcttttgaaactgtaaggctggagtgcaggaggggtgagtggaattcctagtgtagcggtgaaatgcgtagatattaggaggaacaccagtggcgaaggcggctcactggactgtaactgacactgaggctcgaaagcgtggggagcaaacaggattagataccctggtagtccacgccgtaaacgatgaatactaggtgtcggggcccataagggcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgaccggacagtaatgtgtcctttccttcgggacagtggagacaggtggtgcatggttgtcgtcagctcgtgtcgtgagatgttgggttaagtcccgcaacgagcgcaacccctatccttagtagccagcagtaagatgggcactctagggagactgccagggataacctggaggaaggtggggatgacgtcaaatcatcatgccccttatgacttgggctacacacgtgctacaatggcgtaaacaaagtgaagcgaagtcgtgaggccaagcaaatcacaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcgaaaatgcccgaagtcggtgacctaacgtaagaaggagccgccgaaggcaggtttgataactggggtgaagtcgtaacaaggtagccgtatcggaaggtgcggctggatcacctcctttctaaggaa

SEQ ID NO: 80 - DNA, 1461 bp, Fusobacterium prausnitzii
  misc_feature: Fusobacterium prausnitzii ATCC 27768
gatcctggctcaggcgaacgctggcggcgcgcctaacacatgcaagtcgaacgagcgagagagagcttgctttctcaagcgagtggcgaacgggtgagtaacgcgtgaggaacctgcctcaaagagggggacaacagttggaaacgactgctaataccgcataagcccacgacccggcatcgggtagagggaaaaggagcaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttgaggaagataatgacggtactcaacaaggaagtgacggctaactacgtgccagcagccgcggtaaaacgtaggtcacaagcgttgtccggaattactgggtgtaaagggagcgcaggcgggaaggcaagttggaagtgaaatccatgggctcaacccatgaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacactgtggccgatgtttactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcctgcgacgcacatagaaatatgtgtttccttcgggacgcagagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgcagatcagcatgctgcggtgaatacgttcccgggccttgtacacaccgcccgtcacaccatgagagccggggggacccgaagtcggtagtctaaccgcaaggaggacgccgccgaaggtaaaactggtgattggggtgaagtcgtaacaaggta

SEQ ID NO: 81 - DNA, 1526 bp, Howardella ureilytica
  misc_feature: Howardella ureilytica GPC 589
agagtttgatcctggctcaggatgaacgctggcggcgtgcttaacacatgcaagtcgaacggagtttataagacagattccttcgggatgaagacttataaacttagtggcggacgggtgagtaacgcgtgggtaacctgccttacacagggggatagcagttggaaacgactggtaataccgcataccttgcattagttgcatgacttatgcaagaaagatttatcggtgtaagatggacccgcgtctgattagctagttggtacggtaacggcgtaccaaggcgacgatcagtagccggcctgagagggtgaacggccacattgggactgagatacggcccagactcctacgggaggcagcagtggggaatattgcacaatggaggcaactctgatgcagcgacgccgcgtgagcgaagaagtatttcggtatgtaaagctctatcagtagggaagataatgacggtacctacagaagaagccccggctaaatacgtgccagcagccgcggtaatacgtatggggcaagcgttatccggatttactgggtgtaaagggagtgtaggcggcagtacaagtcaggagtgaaaacttggggctcaaccccaagactgcttttgaaactgtacagctagagtgtaggaagggcaagcggaattcctggtgtagcggtgaaatgcgtagatatcaggaagaacaccggtggcgaaggcggcttgctggactataactgacgctgagactcgaaagcgtggggagcgaacaggattagataccctggtagtccacgctgtaaacgatgaatactaggtgttgggtctcgatagagattcggtgccgaagcaaacgcattaagtattccacctggggagtacgttcgcaagaatgaaactcaaaggaattgacggggacccgcacaagcggtggagcatgtggtttaattcgaagcaacgcgaagaaccttaccaggtcttgacatcctgctgcccggtcagtaatgtgaccttttcttcggaacagcagagacaggtggtgcatggttgtcgtcagctcgtgtcgtgagatgttgggttaagtcccgcaacgagcgcaaccctcatctatagtagccagcatttcggatgggcactctatagagaccgccggggataacccggaggaggatggggatgacgtcaaatcatcatgccccttatggcctgggctacacacgtgctacaatggcactaaacaaagtgaggcgagacagtgatgttaagcagagcacaaaaacagtgtcccagttcggattgtagtctgcaactcgactacatgaagccggaatcgctagtaatcgcagatcagcatgctgcggtgaatacgttcccgggtcttgtacacaccgcccgtcacaccatgggagtcggaaacgcccgaagccggcggcctaaccgtaaggaaggagccgtcgaaggcgggttcggtgactggggtgaagtcgtaacaaggtagccgtatcggaaggtgcggctggatcacctcctt

SEQ ID NO: 82 - DNA, 1417 bp, Megasphaera elsdenii
  misc_feature: Megasphaera elsdenii LC1
agagtttgatcctggctcaggacgaacgctggcggcgtgcttaacacatgcaagtcgaacgagaagagatgagaagcttgcttcttatcgattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtaggggaatcttccgcaatggacgaaagtctgacggagcaacgccgcgtgaacgatgacggccttcgggttgtaaagttctgttatacgggacgaatggcgtacgggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgagggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcgagttaagtcggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccg

SEQ ID NO: 83 - DNA, 1568 bp, Megasphaera elsdenii
  misc_feature: Megasphaera elsdenii LC1
atatcatggagagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacctcctttct

SEQ ID NO: 84 - DNA, 1568 bp, Megasphaera elsdenii
  misc_feature: Megasphaera elsdenii LC1
atggagagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctatggatagagatatccagttcctcttcggaggacaagaaaacaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccttttggagccagccgtcgaaggtgggggcgatgattggggtgaagtcgtaacaaggtagccgtatcggaaggtgcggctggatcacctcctttctaggga

SEQ ID NO: 85 - DNA, 1568 bp, Megasphaera elsdenii; identical to SEQ ID NO: 84
  misc_feature: Megasphaera elsdenii LC1

SEQ ID NO: 86 - DNA, 1568 bp, Megasphaera elsdenii; identical to SEQ ID NO: 84
  misc_feature: Megasphaera elsdenii LC1

SEQ ID NO: 87 - DNA, 1568 bp, Megasphaera elsdenii; identical to SEQ ID NO: 84
  misc_feature: Megasphaera elsdenii LC1

SEQ ID NO: 88 - DNA, 1568 bp, Megasphaera elsdenii; identical to SEQ ID NO: 84
  misc_feature: Megasphaera elsdenii LC1

SEQ ID NO: 89 - DNA, 1568 bp, Megasphaera elsdenii; identical to SEQ ID NO: 84
  misc_feature: Megasphaera elsdenii LC1

SEQ ID NO: 90 - DNA, 1568 bp, Megasphaera elsdenii; identical to SEQ ID NO: 84
  misc_feature: Megasphaera elsdenii LC1

SEQ ID NO: 91 - DNA, 1492 bp, Parabacteroides johnsonii
  misc_feature: Parabacteroides johnsonii M-165
agagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggtcttctagcaatagctgattacgaggtgctgcatggttgtcgtcagctcgtgccgtgaggtgtcggcttaagtgccataacgagcgcaacccttatcactagttactaacaggtcaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtaacc

SEQ ID NO: 92 - DNA, 1546 bp, Parabacteroides johnsonii
  misc_feature: Parabacteroides johnsonii M-165
caatttcaacaacgaagagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctttctggagc

SEQ ID NO: 93 - DNA, 1530 bp, Parabacteroides johnsonii
  misc_feature: Parabacteroides johnsonii M-165
acaacgaagagtttgatcctggctcaggatgaacgctagcgacaggcttaacacatgcaagtcgaggggcagcatggtaagtagcaatacttattgatggcgaccggcgcacgggtgagtaacgcgtatgcaacttacctatcagagggggatagcccggcgaaagtcggattaatactccataaaacaggggttccgcatgggactatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggatgaaggatctatggtttgtaaacttcttttataggggaataaagtgtgggacgtgttccattttgtatgtaccctatgaataagcatcggctaactccgtgccagcagccgcggtaatacggaggatgcgagcgttatccggatttattgggtttaaagggtgcgtaggtggtaatttaagtcagcggtgaaagtttgtggctcaaccataaaattgccgttgaaactgggttacttgagtgtgtttgaggtaggcggaatgcgtggtgtagcggtgaaatgcatagatatcacgcagaactccaattgcgaaggcagcttactaaaccataactgacactgaagcacgaaagcgtgggtatcaaacaggattagataccctggtagtccacgcagtaaacgatgattactaggagtttgcgatacacagtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtcagaccgaccttgaaagaggttttctagcaatagctgattacgaggtgctgcatggttgtcgtcagctcgtgccgtgaggtgtcggcttaagtgccataacgagcgcaacccttatcactagttactaacaggttaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctctaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggtagccgtaccggaaggtgcggctggaacacctcctt

SEQ ID NO: 94 - DNA, 1530 bp, Parabacteroides johnsonii; identical to SEQ ID NO: 93
  misc_feature: Parabacteroides johnsonii M-165

SEQ ID NO: 95 - DNA, 1662 bp, Phascolarctobacterium
  misc_feature: Phascolarctobacterium sp. YIT 12067
ctttattggagagtttgatcctggctcaggacgaacgctggcggcatgcctaacacatgcaagtcgaacggagaaagttcaacaccaagtatttcatccgctgaagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtgaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaaggtgcggctggatcacctcctttct

SEQ ID NO: 96 - DNA, 1667 bp, Phascolarctobacterium
  misc_feature: Phascolarctobacterium sp. YIT 12067
tttattggagagtttgatcctggctcaggacgaacgctggcggcatgcctaacacatgcaagtcgaacggagaaagttcaacaccaagtatttcatccgctgaagtgtagcggtaaaaattgcgaagcaatttttactacgcattaaaagcatgaactaacacggtggttgaagtattaggtgttgaactttcttagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtgaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaaggtgcggctggatcacctcctttctatggag

SEQ ID NO: 97 - DNA, 1444 bp, Phascolarctobacterium
  misc_feature: Phascolarctobacterium sp. YIT 12067
tagtggcgaacgggtgagtaacgcgtgggcaacctgccctctagatggggacaacatcccgaaaggggtgctaataccgaatgtgacagcaatctcgcatgaggatgctgtgaaagatggcctctatttataagctatcgctagaggatgggcctgcgtctgattagctagttggtggggtaacggcctaccaaggcgatgatcagtagccggtctgagaggatgaacggccacattgggactgagacacggcccagactcctacgggaggcagcagtggggaatcttccgcaatgggcgaaagcctgacggagcaatgccgcgtgagtgatgaaggaattcgttccgtaaagctcttttgtttatgacgaatgtgcagattgtaaataatgatctgtaatgacggtagtaaacgaataagccacggctaactacgtgccagcagccgcggtaatacgtaggtggcgagcgttgtccggaattattgggcgtaaagagcatgtaggcggttttttaagtctggagtgaaaatgcggggctcaaccccgtatggctctggatactggaagacttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacgtccgcaaggatgaaactcaaaggaattgacgggggcccgcacaagcggtggagtatgtggtttaattcgacgcaacgcgaagaaccttaccaaggcttgacattgaatgaccgctccagagatggagctttcccttcggggacatgaaaacaggtggtgcatggctgtcgtcagctcgtgtcgtgagatgttgggttaagtcccgcaacgagcgcaacccctatcctatgttaccagcgggtaatgccggggactcataggagactgccaaggacaacttggaggaaggcggggatgacgtcaagtcatcatgccccttatgtcttgggctacacacgtactacaatggtcggcaacagagggaagcaaagccgtgaggcagagcaaaccccagaaacccgatcccagttcggattgcaggctgcaactcgcctgcatgaagtcggaatcgctagtaatcgcaggtcagcatactgcggtgaatacgttcccgggccttgtacacaccgcccgtcacaccacgaaagttggtaacacccgaagccggtggggtaaccgtaaggagccagccgtctaaggtggggccgatgattggggtgaagtcgtaacaaggtagccgtatcggaagg

SEQ ID NO: 98 - DNA, 1662 bp, Phascolarctobacterium; identical to SEQ ID NO: 95
  misc_feature: Phascolarctobacterium sp. YIT 12067

SEQ ID NO: 99 - DNA, 1480 bp, Roseburia hominus
  misc_feature: Roseburia hominus A2-183
gatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgcggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacagagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaagg

SEQ ID NO: 100 - DNA, 1536 bp, Roseburia hominus
  misc_feature: Roseburia hominus A2-183
tataacatgagagtttgatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccggtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacagagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaaggtagccgtatcggaaggtgcggctggatcacctcctttct

SEQ ID NO: 101 - DNA, 1540 bp, Roseburia hominus
  misc_feature: Roseburia hominus A2-183
taacatgagagtttgatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacaaagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaaggtagccgtatcggaaggtgcggctggatcacctcctttctaaggaa

SEQ ID NO: 102 - DNA, 1540 bp, Roseburia hominus
  misc_feature: Roseburia hominus A2-183
taacatgagagtttgatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccagtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacagagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaaggtagccgtatcggaaggtgcggctggatcacctcctttctaaggaa

SEQ ID NO: 103 - DNA, 1540 bp, Roseburia hominus; identical to SEQ ID NO: 102
  misc_feature: Roseburia hominus A2-183

SEQ ID NO: 104 - DNA, 1540 bp, Roseburia hominus
  misc_feature: Roseburia hominus A2-183
taacatgagagtttgatcctggctcaggatgaacgctggcggcgtgcttaacacatgcaagtcgaacgaagcactttaattgatttcttcggaatgaagtttttgtgactgagtggcggacgggtgagtaacgcgtgggtaacctgcctcatacagggggataacagttggaaacgactgctaataccgcataagcgcacaggattgcatgatccggtgtgaaaaactccggtggtatgagatggacccgcgtctgattagccagttggcggggtaacggcccaccaaagcgacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaagaatgacggtacctgactaagaagcaccggctaaatacgtgccagcagccgcggtaatacgtatggtgcaagcgttatccggatttactgggtgtaaagggagcgcaggcggtacggcaagtctgatgtgaaatcccggggctcaaccccggtactgcattggaaactgtcggactagagtgtcggaggggtaagtggaattcctagtgtagcggtgaaatgcgtagatattaggaggaacaccagtggcgaaggcggcttactggacgattactgacgctgaggctcgaaagcgtggggagcaaacaggattagataccctggtagtccacgccgtaaacgatgaatactaggtgtcggggagcattgctcttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccactgacagagtatgtaatgtactttctcttcggagcagtggtgacaggtggtgcatggttgtcgtcagctcgtgtcgtgagatgttgggttaagtcccgcaacgagcgcaacccctattcttagtagccagcggttcggccgggcactctagggagactgccagggataacctggaggaaggtggggatgacgtcaaatcatcatgccccttatgacttgggctacacacgtgctacaatggcgtaaacaaagggaagcaatcccgcgagggggagcaaatctcaaaaataacgtctcagttcggactgtagtctgcaactcgactacacgaagctggaatcgctagtaatcgcgaatcagaatgtcgcggtgaatacgttcccgggtcttgtacacaccgcccgtcacaccatgggagttggtaatgcccgaagtcagtgacccaaccgcaaggagggagctgccgaaggcaggactgataactggggtgaagtcgtaacaaggtagccgtatcggaaggtgcggctggatcacctcctttctaaggaa

SEQ ID NO: 105 - DNA, 1370 bp, Ruminococcus callidus
  misc_feature: Ruminococcus callidus VPI 57-31
taacacatgcaagtcgaacggagaacattgagcttgcttaatgttcttagtggcggacgggtgagtaacacgtgagtaacctgcctctgagagtgggatagcttctggaaacggatggtaataccgcataacatcatggattcgcatgtttctgtgatcaaagatttatcgcttagagatggactcgcgtctgattagctagttggtaaggtaacggcttaccaaggcgacgatcagtagccggactgagaggttgatcggccacattgggactgagacacggcccagactcctacgggaggcagcagtggggaatattgcacaatgggggaaaccctgatgcagcgatgccgcgtggaggaagaaggttttcggattgtaaactcctgttgaagaggacgataatgacggtactcttttagaaagctccggctaactacgtgccagcagccgcggtaatacgtagggagcgagcgttgtccggaattactgggtgtaaagggagcgtaggcgggatggcaagtcagatgtgaaaactatgggctcaacccatagactgcatttgaaactgttgttcttgagtgaggtagaggtaagcggaattcctggtgtagcggtgaaatgcgtagagatcaggaggaacatcggtggcgaaggcggcttactgggcctttactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgagtgacgtacctagagataggtattttcttcggaacacaaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggctacacacgtactacaatggcaatataacagagggaagcaatacagcgatgtggagcaaatccccaaaaattgtcccagttcagattgcaggctgcaactcgcctgcatgaagtcggaattgctagtaatcgcagatcagcatgctgcggtgaatacgttcccgggccttgtacacaccgcccgtcacaccatgggagtcggtaacacccaaagccggtcgtctaacctt

SEQ ID NO: 106 - DNA, 1517 bp, Ruminococcus callidus
  misc_feature: Ruminococcus callidus VPI 57-31
ttaaataaagagtttgatcctggctcaggacgaacgctggcggcacgcttaacacatgcaagtcgaacggagaacattgagcttgcttaatgttcttagtggcggacgggtgagtaacacgtgagtaacctgcctctgagagtgggatagcttctggaaacggatggtaataccgcataacatcatggattcgcatgtttctgtgatcaaagatttatcgcttagagatggactcgcgtctgattagctagttggtaaggtaacggcttaccaaggcgacgatcagtagccggactgagaggttgatcggccacattgggactgagacacggcccagactcctacgggaggcagcagtggggaatattgcacaatgggggaaaccctgatgcagcgatgccgcgtggaggaagaaggttttcggattgtaaactcctgttgaagaggacgataatgacggtactcttttagaaagctccggctaactacgtgccagcagccgcggtaatacgtagggagcgagcgttgtccggaattactgggtgtaaagggagcgtaggcgggatggcaagtcagatgtgaaaactatgggctcaacccatagactgcatttgaaactgttgttcttgagtgaggtagaggtaagcggaattcctggtgtagcggtgaaatgcgtagagatcaggaggaacatcggtggcgaaggcggcttactgggcctttactgacgctgaggctcgaaagcgtggggagcaaacaggattagataccctggtagtccacgccgtaaacgatgattactaggtgtggggggactgaccccttccgtgccgcagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcgagtgacgtacctagagataggtattttcttcggaacacaaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggctacacacgtactacaatggcaatataacagagggaagcaatacagcgatgtggagcaaatccccaaaaattgtcccagttcagattgcaggctgcaactcgcctgcatgaagtcggaattgctagtaatcgcagatcagcatgctgcggtgaatacgttcccgggccttgtacacaccgcccgtcacaccatgggagtcggtaacacccaaagccggtcgtctaaccttcgggaggatgccgtctaaggtgggattgatgactggggtgaagtcgtaacaaggtagccgtatcggaaggtgcggctggatcacctcctttctaagga

SEQ ID NO: 107 - DNA, 1545 bp, Acinetobacter johnsonii
  misc_feature: Acinetobacter johnsonii B8
tttaactgaagagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctaccggggttaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 108 - DNA, 1545 bp, Acinetobacter johnsonii
  misc_feature: Acinetobacter johnsonii B8
tttaactgaagagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctacttggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 109 - DNA, 1545 bp, Acinetobacter johnsonii
  misc_feature: Acinetobacter johnsonii B8
tttaactgaagagtttgatcatggctcagattgaacgctggcggcaggcttaacacatgcaagtcgagcggggagatgtagcttgctacatttcctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctaccgaaattaatactttaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 110 - DNA, 1545 bp, Acinetobacter johnsonii
  misc_feature: Acinetobacter johnsonii B8
tttaactgaagagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctaccgagattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 111 - DNA, 1545 bp, Acinetobacter johnsonii
  misc_feature: Acinetobacter johnsonii B8
tttaactgaagagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctactttgattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 112 - DNA, 1545 bp, Acinetobacter johnsonii; identical to SEQ ID NO: 110
  misc_feature: Acinetobacter johnsonii B8

SEQ ID NO: 113 - DNA, 1213 bp, Acinetobacter johnsonii
  misc_feature: Acinetobacter johnsonii B8
tttaactgaagagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctacttggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacg

SEQ ID NO: 114 - DNA, 1545 bp, Acinetobacter johnsonii; identical to SEQ ID NO: 108
  misc_feature: Acinetobacter johnsonii B8

SEQ ID NO: 115 - DNA, 1460 bp, Acinetobacter johnsonii
  misc_feature: Acinetobacter johnsonii B8
  misc_feature (578)..(578): n is a, c, g, or t
  misc_feature (763)..(764): n is a, c, g, or t
  misc_feature (1384)..(1384): n is a, c, g, or t
aacgctggcggcaggcttaacacatgcaagtcgagcggggagaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctacttggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatcccngagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctnntagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggantttgttgcaccagaagtaggtagtctaaccgcaaggaggacgcttaccacggtgtggccgatgactggggtgaagt

SEQ ID NO: 116 - DNA, 1417 bp, Acinetobacter johnsonii
  misc_feature: Acinetobacter johnsonii B8
  misc_feature (170)..(170): n is a, c, g, or t
  misc_feature (1367)..(1367): n is a, c, g, or t
cctggctcagattgaacgctggcggcaggcttaacacatgcaagtcgagcggggagaggtagcttgctacctaacctagcggcggacgggtgagtaatgcttaggaatctgcctattagtgggggacaacattccgaaaggaatgctaataccgcatacgtcctacgggngaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttttggttgtaaagcactttaagcgaggaggaggctacttggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggncttgtacacaccgcccgtcacaccatgggagtttgttgcaccagaagtag

SEQ ID NO: 117 - DNA, 1530 bp, Acinetobacter johnsonii
  misc_feature: Acinetobacter johnsonii B8
agagtttgatcatggctcagattgaacgctggcggcaggcttaacacatgcaagtcgagcggggaaaggtagcttgctacctaacctagcggcggacgggtgagtaatgcttaggaatctgcctattagtgggggacaacattccgaaaggaatgctaataccgcatacgccctacgggggaaagcaggggatcttcggaccttgcgctaatagatgagcctaagtcagattagctagttggtggggtaaaggcctaccaaggcgacgatctgtagcgggtctgagaggatgatccgccacactgggactgagacacggcccagactcctacgggaggcagcagtggggaatattggacaatgggcgaaagcctgatccagccatgccgcgtgtgtgaagaaggccttttggttgtaaagcactttaagcgaggaggaggctacttggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccctgagcttaacttaggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggaatttgttgcaccagaagtaggtagtctaaccgcaaggaggacgcttaccacggtgtggccgatgactggggtgaagtcgtaacaaggtagccgtaggggaacctgcggctggatcacctcctt

SEQ ID NO: 118 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttttggttgtaaagcactttaagcgaggaggaggctacctggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctggagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtagaaactttccagagatggattggtgccttcgggaatttagatacaggtgctgcatggctgtcgtcagctcgtgtcgtgagatgttgggttaagtcccgcaacgagcgcaacccttttccttatttgccagcgagtaaagtcgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgtaaggaggacgcttaccacggtgtggccgatgactggggtgaagtcgtaacaaggtagccgtaggggaacctgcggctggatcacctccttaacgaa

SEQ ID NO: 119 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttatggttgtaaagcactttaagcgaggaggaggctacctagattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 120 - DNA, 1417 bp, Acinetobacter
  misc_feature: Acinetobacter sp. LMG 995
  misc_feature (321)..(321): n is a, c, g, or t
cctggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctncgggaggcagcagtggggaatattggacaatgggcggaagcctgatccagccatgccgcgtgtgtgaagaagcccttatggttgtaaagcactttaagcgaggaggagcctacctagattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtgcgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactaagaactttccagagatggattggtgccttcgggaatctagatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtag

SEQ ID NO: 121 - DNA, 1536 bp, Acinetobacter
  misc_feature: Acinetobacter sp. LMG 995
aactgaagagtttgatcatggctcagattgaacgctggcggcaggcttaacacatgcaagtcgagcgggggaaggtagcttgctactggacctagcggcggacgggtgagtaatgcttaggaatctgcctattagtgggggacaacatctcgaaagggatgctaataccgcatacgtcctacgggagaaagcaggggatcttcggaccttgcgctaatagatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatggggggaaccctgatccagccatgccgcgtgtgtgaagaaggccttatggttgtaaagcactttaagcgaggaggaggctactctagttaatacctagggatagtggacgttactcgcagaataagcaccggctaactctgtgccagcagccgcggtaatacagagggtgcgagcgttaatcggatttactgggcgtaaagcgtgcgtaggcggcttattaagtcggatgtgaaatccccgagcttaacttgggaattgcattcgatactggtgagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttacttgccagcatttcggatgggaactttaaggatactgccagtgacaaactggaggaaggcggggacgacgtcaagtcatcatggcccttacggccagggctacacacgtgctacaatggtcggtacaaagggttgctacacagcgatgtgatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtagctagcctaactgcaaagagggcggttaccacggtgtggccgatgactggggtgaagtcgtaacaaggtagccgtaggggaacctgcggctggatcacctcctta

SEQ ID NO: 122 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttttggttgtaaagcactttaagcgaggaggaggctacctggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgtaaggaggacgcttaccacggtgtggccgatgactggggtgaagtcgtaacaaggtagccgtaggggaacctgcggctggatcacctccttaacgaa

SEQ ID NO: 123 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttttggttgtaaagcactttaagcgaggaggaggctacttagattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctggagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 124 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttatggttgtaaagcactttaagcgaggaggaggctacttagattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttatttgccagcgagtaatgtcgggaactttaaggatactgccagtgacaaactggaggaaggcggggacgacgtcaagtcatcatggcccttacggccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgtaaggaggacgcttaccacggtgtggccgatgactggggtgaagtcgtaacaaggtagccgtaggggaacctgcggctggatcacctccttaacgaa

SEQ ID NO: 125 - DNA, 1545 bp, Acinetobacter
  misc_feature: Acinetobacter sp. LMG 995
ttaaactgaagagtttgatcatggctcagattgaacgctggcggcaggcttaacacatgcaagtcgagcggggaagggtagcttgctacctaacctagcggcggacgggtgagtaatgcttaggaatctgccatttagtgggggacaacattccgaaaggaatgctaataccgcatacgtcctacgggagaaagcaggggatcttcggaccttgcgctaaatgatgagcctaagtcggattagctagttggtggggtaaaggcctaccaaggcgacgatctgtagcgggtctgagaggatgatccgccacactgggactgagacacggcccagactcctacgggaggcagcagtggggaatattggacaatgggcggaagcctgatccagccatgccgcgtgtgtgaagaaggccttttggttgtaaagcactttaagcgaggaggaggctacctggattaatactctaggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctggagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggtcttgacatagtaagaactttccagagatggattggtgccttcgggaacttacatacaggtgctgcatggctgtcgtcagctcgtgtcgtgagatgttgggttaagtcccgcaacgagcgcaacccttttccttatttgccagcgggttaagccgggaactttaaggatactgccagtgacaaactggaggaaggcggggacgacgtcaagtcatcatggcccttacgaccagggctacacacgtgctacaatggtcggtacaaagggttgctacctagcgataggatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgcttaccacggtgtggccgatgactggggtgaagtcgtaacaaggtagccgtaggggaacctgcggctggatcacctccttaacgaa

SEQ ID NO: 126 - DNA, 1502 bp, Alicycliphilus denitrificans
  misc_feature: Alicycliphilus denitrificans K601
cctggctcagattgaacgctggcggcatgccttacacatgcaagtcgaacggtaacaggtcttcggatgctgacgagtggcgaacgggtgagtaatacatcggaacgtgcccgatcgtgggggataacgaagcgaaagctttgctaataccgcatacgatctacggatgaaagcgggggatcttcggacctcgcgcggacggagcggccgatggcagattaggtagttggtgggataaaagcttaccaagccgacgatctgtagctggtctgagaggatgatcagccacactgggactgagacacggcccagactcctacgggaggcagcagtggggaattttggacaatgggcgcaagcctgatccagccatgccgcgtgcaggatgaaggccttcgggttgtaaactgcttttgtacggaacgaaaaggtgccttctaataaagggcgctcatgacggtaccgtaagaataagcaccggctaactacgtgccagcagccgcggtaatacgtagggtgcgagcgttaatcggaattactgggcgtaaagcgtgcgcaggcggttttgtaagacagaggtgaaatccccgggctcaacctgggaactgcctttgtgactgcaaggctggagtgcggcagacggggatggaattccgcgtgtatcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcgactggttgttggggattaattttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtatggaatcccgaagagatttgggagtgctcgaaagagagccataacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggtagccaacccgcgagggggagccaatcccacaaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggtctcgccagaagtgggtagcctaaccgcaaggagggcgcttaccacggcggggttcgtgactggggtgaagtcgtaacaaggtagccgtatcggaaggtgcggctggat

SEQ ID NO: 127 - DNA, 1533 bp, Alicycliphilus denitrificans
  misc_feature: Alicycliphilus denitrificans K601
tcgaactgtagagtttgatcctggctcagattgaacgctggcggcatgccttacacatgcaagtcgaacggtaacaggtcttcggatgctgacgagtggcgaacgggtgagtaatacatcggaacgtgcccgatcgtgggggataacgaagcgaaagctttgctaataccgcatacgatctacggatgaaagcgggggatcttcggacctcgcgcggacggagcggccgatggcagattaggtagttggtgggataaaagcttaccaagccgacgatctgtagctggtctgagaggatgatcagccacactgggactgagacacggcccagactcctacgggaggcagcagtggggaattttggacaatgggcgcaagcctgatccagccatgccgcgtgcaggatgaaggccttcgggttgtaaactgcttttgtacggaacgaaaaggtgccttctaataaagggcgctcatgacggtaccgtaagaataagcaccggctaactacgtgccagcagccgcggtaatacgtagggtgcgagcgttaatcggaattactgggcgtaaagcgtgcgcaggcggttttgtaagacagaggtgaaatccccgggctcaacctgggaactgcctttgtgactgcaaggctggagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttggggattaattttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtatggaatcccgaagagatttgggagtgctcgaaagagagccataacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggtagccaacccgcgagggggagccaatcccacaaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggtctcgccagaagtgggtagcctaaccgcaaggagggcgcttaccacggcggggttcgtgactggggtgaagtcgtaacaaggtagccgtatcggaaggtgcggctggatcacctcctttct

SEQ ID NO: 128 - DNA, 1537 bp, Alicycliphilus denitrificans
  misc_feature: Alicycliphilus denitrificans K601
agatcgaactgtagagtttgatcctggctcagattgaacgctggcggcatgccttacacatgcaagtcgaacggtaacaggtcttcggatgctgacgagtggcgaacgggtgagtaatacatcggaacgtgcccgatcgtgggggataacgaagcgaaagctttgctaataccgcatacgatctacggatgaaagcgggggatcttcggacctcgcgcggacggagcggccgatggcagattaggtagttggtgggataaaagcttaccaagccgacgatctgtagctggtctgagaggatgatcagccacactgggactgagacacggcccagactcctacgggaggcagcagtggggaattttggacaatgggcgcaagcctgatccagccatgccgcgtgcaggatgaaggccttcgggttgtaaactgcttttgtacggaacgaaaaggtgccttctaataaagggcgctcatgacggtaccgtaagaataagcaccggctaactacgtgccagcagccgcggtaatacgtagggtgcgagcgttaatcggaattactgggcgtaaagcgtgcgcaggcggttttgtaagacagaggtgaaatccccgggctcaacctgggaactgcctttgtgactgcaaggctggagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttggggattaattttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatgtatggaatcccgaagagatttgggagtgctcgaaagagagccataacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggctggtacagagggtagccaacccgcgagggggagccaatcccacaaagccagtcgtagtccggatcgcagtctgcaactcgactgcgtgaagtcggaatcgctagtaatcgcggatcagaatgtcgcggtgaatacgttcccgggtcttgtacacaccgcccgtcacaccatgggagcgggtctcgccagaagtgggtagcctaaccgcaaggagggcgcttaccacggcggggttcgtgactggggtgaagtcgtaacaaggtagccgtatcggaaggtgcggctggatcacctcctttctg

SEQ ID NO: 129 - DNA, 1537 bp, Alicycliphilus denitrificans; identical to SEQ ID NO: 128
  misc_feature: Alicycliphilus denitrificans K601

SEQ ID NO: 130 - DNA, 1537 bp, Alicycliphilus denitrificans; identical to SEQ ID NO: 128
  misc_feature: Alicycliphilus denitrificans K601

SEQ ID NO: 131 - DNA, 1416 bp, Aquabacterium parvum
  misc_feature: Aquabacterium parvum B6
  misc_feature (23)..(23): n is a, c, g, or t
  misc_feature (1007)..(1007): n is a, c, g, or t
tgaacgctggcggcatgccttanacatgcaagtcgaacggtaacaggtcttcggatgctgacgagtggcgaacgggtgagtaatgcatcggaacgtgcccagtagtgggggatagcccggcgaaagccggattaataccgcatacgacctgagggtgaaagggggggatcgcaagacctctcgctattggagcggccgatgtcagattagctagttggtggggtaaaggcctaccaaggcaacgatctgtagttggtctgagaggacgaccagccacactgggactgagacacggcccagactcctacgggaggcagcagtggggaattttggacaatgggcgcaagcctgatccagcaatgccgcgtgcaggaagaaggccttcgggttgtaaactgcttttgtcagggaagaaatcttctgggttaatacctcgggaggatgacggtacctgaagaataagcaccggctaactacgtgccagcagccgcggtaatacgtagggtgcgagcgttaatcggaattactgggcgtaaagcgtgcgcaggcggctttgcaagacagaggtgaaatccccgggctcaacctgggaactgcctttgtgactgcaaggctagagtacggcagagggggatggaattccgcgtgtagcagtgaaatgcgtagatatgcggaggaacaccaatggcgaaggcaatcccctgggcctgtactgacgctcatgcacgaaagcgtggggagcaaacaggattagataccctggtagtccacgccctaaacgatgtcaactggttgttggacggcttgctgttcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgctaggaatcttgcagagatgtgagagtgctcgaaagagaacctaganacaggtgctgcatggccgtcgtcagctcgtgtcgtgagatgttgggttaagtcccgcaacgagcgcaacccttgtcatcagttgctacgaaagggcactctgatgagactgccggtgacaaaccggaggaaggtggggatgacgtcaggtcctcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcttgccgcggtgaatacgttcccgggtcttgtacacaccgcccgtcacaccatgggagcgggttctgccagaagtagttagcctaaccgcaaggagg

SEQ ID NO: 132 - DNA, 1489 bp, Bacteroides plebeius
  misc_feature: Bacteroides plebeius M12
agagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcaaaggaatgatctggaaacaggtcagtcttcggacctttgtgaaggtgctgcatggttgtcgtcagctcgtgccgtgaggtgtcggcttaagtgccataacgagcgcaaccctcgccgccagttactaacaggtaatgctgaggactctggcgggactgccatcgtaagatgtgaggaaggtggggatgacgtcaaatcagcacggcccttacgtccggggctacacacgtgttacaatgggcggtacagaaggcagctacctggcgacaggatgccaatcccgaaagccgctctcagttcggactggagtctgcaacccgactccacgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaaccggtaattggggctaagtcgtaacaaggtaacc

SEQ ID NO: 133 - DNA, 1128 bp, Bacteroides plebeius
  misc_feature: Bacteroides plebeius M12
ttttacaatgaagagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttattcgatgatacgcgaggaaccttacccgggcttaattgcaaaggaatgatctgaaacagtcagtcttcggactttgtgaggtgctgcatgtgtcgtcagctcgtgccgtgagtgtcgcttaagtgcaataacgagcgcatccctccgccgccagttacctaacaggtaa

SEQ ID NO: 134 - DNA, 1315 bp, Bacteroides plebeius
  misc_feature: Bacteroides plebeius M12
ttttacaatgaagagtttgatcctggctcaggatgaacgctagctacaggcttaacacatgcaagtcgaggggcagcgggattgaagcttgcttcaattgccggcgaccggcgcacgggtgagtaacgcgtatccaaccttccgtacactcagggatagcctttcgaaagaaagattaatacctgatggtatgatgagattgcatgatagcatcattaaagatttatcggtgtacgatggggatgcgttccattaggtagtaggcggggtaacggcccacctagcctacgatggataggggttctgagaggaaggtcccccacattggaactgagacacggtccaaactcctacgggaggcagcagtgaggaatattggtcaatggacgagagtctgaaccagccaagtagcgtgaaggatgaaggtcctacggattgtaaacttcttttataagggaataaaacctcccacgtgtgggagcttgtatgtaccttatgaataagcatcggctaactccgtgccagcagccgcggtaatacggaggatgcgagcgttatccggatttattgggtttaaagggagcgcagacgggtcgttaagtcagctgtgaaagtttggggctcaaccttaaaattgcagttgatactggcgtccttgagtgcggttgaggtgtgcggaattcgtggtgtagcggtgaaatgcttagatatcacgaagaactccgattgcgaaggcagcacactaagccgtaactgacgttcatgctcgaaagtgtgggtatcaaacaggattagataccctggtagtccacacggtaaacgatggatactcgctgttggcgatatacagtcagcggcttagcgaaagcgttaagtatcccacctggggagtacgccggcaacggtgaaactcaaaggaattgacgggggcccgcacaagcggaggaacatgtggtttaattcgatgatacgcgaggaaccttacccgggcttaaattgcaaaggaatgatctggaaacaggtcagtcttcggacctttgtgaaggtgctgcatggttgtcgtcagctcgtgccgtgaggtgtcggcttaagtgccataacgagcgcaaccctcgccgccagttactaacaggtaatgctgaggactctggcgggactgccatcgtagatgtgaggaaggtggggatgacgtcaatcagcacggcccttacgtccggggctacacacgtgttacaatgggcggtacagagcagctactgcgacagatgcatccgaagccgcttctcagttcggactggagttctgcaacccgact

SEQ ID NO: 135 - DNA, 1537 bp, Bacteroides
  misc_feature: Bacteroides sp 2_2_4
cttttacaatgaagagtttgatcctggctcaggatgaacgctagctacaggcttaacacatgcaagtcgaggggcagcatttcgctagcaatagtgaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactgggagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaataatctggaaacaggttagccgcaaggcatttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacctcctttctggagc

SEQ ID NO: 136 - DNA, 1525 bp, Bacteroides
  misc_feature: Bacteroides sp 2_2_4
tacaatgaagagtttgatcctggctcaggatgaacgctagctacaggcttaacacatgcaagtcgaggggcagcatttcgctagcaatagtgaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactgggagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaataatctggaaacaggttagccgcaaggcatttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacctcctt

SEQ ID NO: 137 - DNA, 1533 bp, Dechloromonas
  misc_feature: Dechloromonas sp. HZ
agagtttgatcctggctcagattgaacgctggcggcatgccttacacatgcaagtcgaacggcagcacgggcttcggtctggtggcgagtggcgaacgggtgagtaatgtatcggaacgtacctttcagtgggggataacgtagcgaaagttacgctaataccgcatattctgtgagcaggaaagcaggggatcgcaagaccttgcgctgattgagcggccgatatcagattagctagttggtggggtaaaggcctaccaaggctacgatctgtagcgggtctgagaggatgatccgccacactggaactgagacacggtccagactcctacgggaggcagcagtggggaattttggacaatgggggcaaccctgatccagccatgccgcgtgagtgaagaaggccttcgggttgtaaagctctttcggccgggaagaaatcgcataggttaataccctgtgtggatgacggtaccggaataagaagcaccggctaactacgtgccagcagccgcggtaatacgtagggtgcgagcgttaatcggaattactgggcgtaaagcgtgcgcaggcggtttcgtaagacagaggtgaaatccccgggctcaacctgggaactgcctttgtgactgcgaagctagagtacggcagaggggggtggaattccacgtgtagcagtgaaatgcgtagagatgtggaggaacaccgatggcgaaggcagccccctgggccgatactgacgctcatgcacgaaagcgtgggtagcaaacaggattagataccctggtagtccacgccctaaacgatgtcaactaggtgttgggtgggtaaaaccatttagtaccgtagctaacgcgtgaagttgaccgcctggggagtacggccgcaaggttaaaactyaaatgaattgacggggacccgcacaagcggtggatgatgtggattaattcgatgcaacgcgaaaaaccttacctacccttgacatgttcagaatcccggagagatttgggagtgcccgaaagggagctggaacacaggtgctgcatggctgtcgtcagctcgtgtcgtgagatgttgggttaagtcccgcaacgagcgcaacccttgtcgttaattgccatcatttagttgggcactttaacgagactgccggtgacaaaccggaggaaggtggggatgacgtcaagtcctcatggcccttatgggtagggcttcacacgtcatacaatggtcggtacagagggttgccaagccgcgaggtggagccaatcccagaaagccgatcgtagtccggattgcaggctgcaactcgcctgcatgaagtcggaatcgctagtaatcgcggatcagcatgtcgcggtgaatacgttcccgggtcttgtacacaccgcccgtcacaccatgggagcgggttccgccagaagtaggtagcctaaccgcaaggggggcgcttaccatggcggggttcgtgactggggtgaagtcgtaacaaggtagccgtaggggaacctgcggctggatcacctcctttct

SEQ ID NO: 138 - DNA, 1530 bp, Dialister invisus
  misc_feature: Dialister invisus E7.25
agagtttgatcctggctcaggacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtaacc

SEQ ID NO: 139 - DNA, 1574 bp, Dialister invisus
  misc_feature: Dialister invisus E7.25
atggagagtttgatcctggctcaggacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgcggctggatcacctcctttctaaggaga

SEQ ID NO: 140 - DNA, 1575 bp, Dialister invisus
  misc_feature: Dialister invisus E7.25
atggagagtttgatcctggctcaggacgaacgctggcggcgtgcttaacacatgcaagtcgaacgaaaagagggaaagagcttgctctttccggaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgcggctggatcacctcctttctaaggaga

SEQ ID NO: 141 - DNA, 1575 bp, Dialister invisus
  misc_feature: Dialister invisus E7.25
atggagagtttgatcctggctcaggacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaatgccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgcggctggatcacctcctttctaaggaga

SEQ ID NO: 142 - DNA, 1567 bp, Dialister invisus
  misc_feature: Dialister invisus E7.25
tggagagtttgatcctggctcaggacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaatgccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgcggctggatcacctcctttct

SEQ ID NO: 143 - DNA, 1524 bp, Dialister invisus
  misc_feature: Dialister invisus E7.25
gacgaacgctggcggcgtgcttaacacatgcaagtcgaacgaaaagacggaaagagcttgctcttttcagaattgagtggcaaacgggtgagtaacacgtaaacaacctgccttcaggatggggacaacagacggaaacgactgctaataccgaataagttccaagagccgcatggcccatggaagaaaaggtggcctctacctgtaagctatcgcctgaagaggggtttgcgtctgattagctggttggaggggtaacggcccaccaaggcgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaaactctgtgatccgggacgaaaaggcagagtgcgaagaacaaactgcattgacggtaccggaaaagcaagccacggctaactacgtgccagcagccgcggtaatacgtaggtggcaagcgttgtccggaattattgggcgtaaagcgcgcgcaggcggcttcccaagtccctcttaaaagtgcggggcttaaccccgtgatgggaaggaaactgggaagctggagtatcggagaggaaagtggaattcctagtgtagcggtgaaatgcgtagagattaggaagaacaccggtggcgaaggcgactttctggacgaaaactgacgctgaggcgcgaaagcgtggggagcaaacaggattagataccctggtagtccacgccgtaaacgatggatactaggtgtaggaggtatcgacccctcctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaggtcttgacattgatcgcgatctgcagaaatgcggagttcttcttcggaagacgagaaaacaggtggtgcacggctgtcgtcagctcgtgtcgtgagatgttgggttaagtcccgcaacgagcgcaacccctatcatttgttaccagcacgtaaaggtggggactcaaatgagaccgccgcggacaacgcggaggaaggcggggacgacgtcaagtcatcatgccccttatgacctgggctacacacgtactacaatgggtgtcaacaaagagaagcgaacccgcgaggaagagcaaacctcaaaaacacacccccagttcagatcgcaggctgcaacccgcctgcgtgaagtaggaatcgctagtaatcgcgggtcagcataccgcggtgaatacgttcccgggccttgtacacaccgcccgtcacactatgagagtcagaaacacccgaagccggtgaggtaaccgcaaggagccagccgtcgaaggcggagctgatgattggagtgaagtcgtaacaaggtagccgtatcggaaggtgc

SEQ ID NO: 144 - DNA, 1567 bp, Dialister invisus; identical to SEQ ID NO: 142
  misc_feature: Dialister invisus E7.25

SEQ ID NO: 145 - DNA, 1487 bp, Micromonas micros
  misc_feature: Micromonas micros ACM 5086
agagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtaacc

SEQ ID NO: 146 - DNA, 1536 bp, Micromonas micros
  misc_feature: Micromonas micros ACM 5086
tttaaatagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctttctaaggag

SEQ ID NO: 147 - DNA, 1521 bp, Micromonas micros
  misc_feature: Micromonas micros ACM 5086
tagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctt

SEQ ID NO: 148 - DNA, 1349 bp, Micromonas micros
  misc_feature: Micromonas micros ACM 5086
agtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagg

SEQ ID NO: 149 - DNA, 1530 bp, Micromonas micros
  misc_feature: Micromonas micros ACM 5086
tttaaatagagagtttgatcctggctcaggacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcgtaacaaggtagccgtatcggaaggtgcggctggatcacctcctttct

SEQ ID NO: 150 - DNA, 1481 bp, Micromonas micros
  misc_feature: Micromonas micros ACM 5086
gacgaacgctggcggcgtgcttaacacatgcaagtcgaacgtgatttttgtggaaattctttcgggaatggaaatgaaatgaaagtggcgaacgggtgagtaacacgtgagcaacctaccttacacagggggatagccgttggaaacgacgattaataccgcatgagaccacagaatcgcatgatataggggtcaaagatttatcggtgtaagaagggctcgcgtctgattagctagttggaagggtaaaggcctaccaaggcgacgatcagtagccggtctgagaggatgaacggccacattggaactgagacacggtccaaactcctacgggaggcagcagtggggaatattgcacaatggggggaaccctgatgcagcgacgccgcgtgagcgaagaaggttttcgaatcgtaaagctctgtcctatgagaagataatgacggtatcataggaggaagccccggctaaatacgtgccagcagccgcggtaatacgtatggggcgagcgttgtccggaattattgggcgtaaagggtacgtaggcggttttttaagtcaggtgtgaaagcgtgaggcttaacctcattaagcacttgaaactggaagacttgagtgaaggagaggaaagtggaattcctagtgtagcggtgaaatgcgtagatattaggaggaataccggtggcgaaggcgactttctggacttttactgacgctcaggtacgaaagcgtggggagcaaacaggattagataccctggtagtccacgccgtaaacgatgaatgctaggtgttgggagtcaaatctcggtgccgaagttaacacattaagcattccgcctggggagtacggtggcaacactgaaactcaaaggaattgacggggacccgcacaagcagcggagcatgtggtttaattcgaagcaacgcgaagaaccttaccaaggcttgacatatagttgagttattgagaaattgataagtccctcgggacaactatacaggtggtgcatggttgtcgtcagctcgtgtcgtgagatgttgggttaagtcccgcaacgagcgcaacccttatcttcagttgccagcacgtagaggtgggaactctggagagactgccgatgacaaatcggaggaaggtggggatgacgtcaaatcatcatgccctttatgtcttgggctacacacgtgctacaatggttggtacaacgagaagcgagatagagatgttaagcgaaactctaaaaaccaatctcagttcggattgtaggctgcaactcgcctacatgaagtcggagttgctagtaatcgcgaatcagaatgtcgcggtgaatgcgttcccgggtcttgtacacaccgcccgtcacaccatgggagttggcaatacccgaagccgccgatctaaccgcaaggaggaaggcgtcgaaggtagggttaatgactggggtgaagtcctaacaaggtagccgtatcggaaggtgc

SEQ ID NO: 151 - DNA, 1455 bp, Sutterella stercoricanis
  misc_feature: Sutterella stercoricanis 5BAC4
ggcggcatgctttacacatgcaagtcgaacggcagcacagggagcttgctcctgggtggcgagtggcgcacgggtgagtaatacatcggaacgtgtcctattgtgggggataactgtcggaaagggtggctaataccgcataagacctgagggtgaaagcgggggatcgcaagacctcgcgcaattggagcggccgatgcccgattagctagttggtgaggtaatagcttaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaaggattgtgctaataccatgatctgctgacggtacctgaagaataagcaccggctaactacgtgccagcagccgcggtaatacgtagggtgcaagcgttaatcggaattactgggcgtaaagcgtgcgcaggcggttctgtaagacagatgtgaaatccccgggctcaacctgggaattgcatttgtgactgcaggactagagttcatcagaggggggtggaattccaagtgtagcagtgaaatgcgtagatatttggaagaacaccaatggcgaaggcagccccctgggatgcgactgacgctcatgcacgaaagcgtggggagcaaacaggattagataccctggtagtccacgccctaaacgatgtctactggttgttggggattaatatccttggtaacgaagctaacgcgtgaagtagaccccctggggagagcggtcgcaagattaaaactcaaaggaattgacggggacccgcacaagcggtggatgatgtggattaattcgatgcaacgcgaaaaaccttacgtagccttgacatgccaggaatcctgaagagatttgggagtgcccgcaagggaatctggacacaggtgctgcatggctgtcgtcagctcgtgtcgtgagatgttgggttaagtcccgcaacgagcgcaacccttgtcattagttgctacgcaagagcactctaatgagactgccggtgacaaaccggaggaaggtggggatgacgtcaagtcctcatggcccttatggctagggcctcacacgtcatacaatggtcggaacagagggcagcgaagccgcgaggtggagcaaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgtctaaccgtaaggaaggacggcgtccacggtgggtttcatgactggggtgaagtcgt

(19) United States
(12) Patent Application Publication
(10) Pub. No.: US 2023/0030056 A1
(43) Pub. Date: Feb. 2, 2023

(54) METHODS AND COMPOSITIONS FOR TREATING ULCERATIVE COLITIS

(71) Applicant: Second Genome, Inc., Brisbane, CA (US)

(72) Inventors: Jayamary Divya Ravichandar, Brisbane, CA (US); Andrew Wonhee Han, Brisbane, CA (US); Todd Zachary DeSantis, Brisbane, CA (US); Cheryl-Emiliane T. Chow, Brisbane, CA (US)

(21) Appl. No.: 17/758,713

(22) PCT Filed: Jan. 15, 2021
(86) PCT No.: PCT/US2021/013756

§ 371 Date: Jul. 13, 2022

Related U.S. Application Data
(60) Provisional application No. 62/962,775, filed on Jan. 17, 2020.

(51) Int. Cl. G01N 33/569

(52) U.S. Cl. CPC G01N 33/56911; G01N 2800/065

(57) ABSTRACT

Methods and compositions are provided herein for treating and/or diagnosing ulcerative colitis in a subject, using one or more bacterial strains such as Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, or Ruminococcus callidus VPI 57-31.

### CROSS-REFERENCE TO RELATED APPLICATIONS

This application claims priority to U.S. Provisional Application No. 62/962,775 filed on Jan. 17, 2020, which is herein incorporated by reference in its entirety.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: 47192_0046P01_ST25.txt, date created, Jan. 17, 2020, file size≈320 kilobytes.

### TECHNICAL FIELD

The present disclosure is related to bacterial strains and compositions thereof, and using such bacterial strains and compositions thereof for treating ulcerative colitis in a subject.

### BACKGROUND

Inflammatory diseases represent a substantial problem in the healthcare industry. For example, chronic inflammatory diseases include many common conditions such as inflammatory bowel disease (IBD), rheumatoid arthritis, restenosis, psoriasis, multiple sclerosis, surgical adhesions, tuberculosis, and chronic inflammatory lung diseases. It is estimated that about 3 million people in the United States currently suffer from IBD alone, and the incidence of IBD has dramatically increased in developed countries in recent years. IBD is characterized by chronic inflammation of the gastrointestinal tract and can lead to stenoses, abscesses, fistulas, extraintestinal manifestations and colitis-associated neoplasias and cancer. Crohn's disease (CD) and ulcerative colitis (UC) are the two main forms of IBD, which have distinct as well as overlapping pathologic and clinical characteristics. It is known that both genetic and environmental factors, including impaired host immune response to the intestinal microbiota, contribute to IBD.

The microbiome of the gastrointestinal tract comprises a diverse array of microorganisms, primarily prokaryotes, which play a significant role in the health of the host organism. The complexity of the microbiome, in terms of both its population makeup and composite function, has recently become an intense area of study as research increasingly shows that manipulation of the microbiome can provide health benefits and may be effective in treating a number of diseases and disorders. Currently, a number of probiotics are marketed which contain live bacteria and yeast and are believed to augment the benefits of these microbes which naturally occur in the human body. Increasingly, live biotherapeutic products (LBPs) are being developed for controlled clinical studies and regulatory approval in the treatment of disease.

Finding a microbe-based therapy for inflammatory diseases such as ulcerative colitis has been challenging. In IBD, for example, this is at least partly because it is a heterogeneous disease with a complex diagnosis, as well as multiple etiologies, pathologies, and treatment responses. Furthermore, finding consistent associations between inflammatory diseases and the microbiome has proven difficult. Improved microbe-based therapies are desirable.

### SUMMARY

Provided herein are methods of identifying a subject as having or having an increased likelihood of developing ulcerative colitis that include a) identifying a subject having a sample that has i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as having or having an increased likelihood of developing ulcerative colitis; or b) identifying a subject having a sample that does not have (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as not having or not having an increased likelihood of developing ulcerative colitis.

Also provided herein are methods of diagnosing a subject as having ulcerative colitis that include a) identifying a subject having a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as having ulcerative colitis; or b) identifying a subject having a sample that does not have (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonasvirosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as not having ulcerative colitis.

Also provided herein are methods of treating ulcerative colitis in a subject that include a) administering a ulcerative colitis therapy to a subject determined to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonasvirosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus; or b) not administering a ulcerative colitis therapy to a subject determined not to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

Also provided herein are methods for treating a subject in need thereof that include a) administering a composition comprising an effective amount of a bacterial species selected from the group consisting of Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus; or b) not administering a composition comprising an effective amount of a bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined not to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

In some embodiments of any of the methods described herein, the subject has ulcerative colitis.

In some embodiments of any of the methods described herein, the method comprises detecting the level of one or more bacterial species in the sample from the subject. In some embodiments of any of the methods described herein, the level of Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, or Sutterella stercoricanis is increased in comparison to the same bacterial species in a reference sample.

In some embodiments of any of the methods described herein, in the method comprises determining that the sample has (i) an increased level of two or more (e.g., three or more, four or more, five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of two or more (e.g., three or more, four or more, five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonasvirosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

In some embodiments of any of the methods described herein, the method comprises determining that the sample has (i) an increased level of three or more (e.g., four or more, five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of three or more (e.g., four or more, five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

In some embodiments of any of the methods described herein, the method comprises determining that the sample has (i) an increased level of four or more (e.g., five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of four or more (e.g., five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

Also provided herein are methods for treating a subject in need thereof that include administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, and a combination thereof. In some embodiments of any of the methods described herein, the subject has ulcerative colitis.

Also provided herein are methods for treating ulcerative colitis in a subject that include administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, and a combination thereof.

Also provided herein are methods for treating ulcerative colitis in a subject that include (a) detecting a dysbiosis associated with ulcerative colitis in a sample from the subject; and (b) administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, and a combination thereof.

In some embodiments of any of the methods described herein, the sample is a biopsy sample. In some embodiments of any of the methods described herein, the sample is a colorectal biopsy sample. In some embodiments of any of the methods described herein, the sample is a fecal sample.

In some embodiments of any of the methods described herein, detecting the dysbiosis associated with ulcerative colitis comprises determining bacterial gene expression in the sample from the subject. In some embodiments of any of the methods described herein, detecting the dysbiosis associated with ulcerative colitis comprises determining bacterial composition in the sample from the subject. In some embodiments of any of the methods described herein, detecting the dysbiosis associated with ulcerative colitis comprises determining that Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, Sutterella stercoricanis, or a combination thereof, is increased (e.g., enriched) in the sample from subject. In some embodiments of any of the methods described herein, detecting the dysbiosis associated with ulcerative colitis comprises determining that Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, or a combination thereof, is decreased in the sample from subject.

In some embodiments of any of the methods described herein, Bacillus sp., Bacteroides coprocola, Bacteroides eggerthii, Bacteroides vulgatus, Butyricimonasvirosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, or a combination thereof, is decreased in the gastrointestinal tract of the subject.

Also provided herein are methods for treating a subject in need thereof that include decreasing a population of an increased (e.g., enriched) bacterial strain in the subject, wherein the increased (e.g., enriched) bacterial strain is selected from the group consisting of Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, Sutterella stercoricanis, and a combination thereof.

In some embodiments of any of the methods described herein, the subject has ulcerative colitis.

In some embodiments of any of the methods described herein, decreasing the population of an increased (e.g., enriched) bacterial strain comprises administering to the subject a bacteriophage. In some embodiments of any of the methods described herein, decreasing the population of an increased (e.g., enriched) bacterial strain comprises administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, Ruminococcus callidus, and a combination thereof.

In some embodiments of any of the methods described herein, the ulcerative colitis is distal colitis. In some embodiments of any of the methods described herein, the ulcerative colitis is extensive colitis. In some embodiments of any of the methods described herein, the ulcerative colitis is selected from the group consisting of: ulcerative proctitis, left-sided colitis, proctosigmoiditis, and pancolitis.

In some embodiments of any of the methods described herein, the bacterial strain comprises Bacillus sp. BT1B_CT2. In some embodiments of any of the methods described herein, the bacterial strain comprises Bacteroides coprocola M16. In some embodiments of any of the methods described herein, the bacterial strain comprises Bacteroides eggerthii CCUG 9559. In some embodiments of any of the methods described herein, the bacterial strain comprises Bacteroides vulgatus 8482. In some embodiments of any of the methods described herein, the bacterial strain comprises Butyricimonas virosa MT12. In some embodiments of any of the methods described herein, the bacterial strain comprises Coprococcus eutactus ATCC 27759. In some embodiments of any of the methods described herein, the bacterial strain comprises Desulfovibrio piger DSM 749. In some embodiments of any of the methods described herein, the bacterial strain comprises Dolosigranulum pigrum IFO 15550. In some embodiments of any of the methods described herein, the bacterial strain comprises Eubacterium biforme DSM 3989. In some embodiments of any of the methods described herein, the bacterial strain comprises Eubacterium eligens DSM 3376. In some embodiments of any of the methods described herein, the bacterial strain comprises Fusobacterium prausnitzii ATCC 27768. In some embodiments of any of the methods described herein, the bacterial strain comprises Howardella ureilytica GPC 589. In some embodiments of any of the methods described herein, the bacterial strain comprises Megasphaera elsdenii LC1. In some embodiments of any of the methods described herein, the bacterial strain comprises Parabacteroides johnsonii M-165. In some embodiments of any of the methods described herein, the bacterial strain comprises Phascolarctobacterium sp. YIT 12067. In some embodiments of any of the methods described herein, the bacterial strain comprises Roseburia hominus A2-183. In some embodiments of any of the methods described herein, the bacterial strain comprises Ruminococcus callidus VPI 57-31.

In some embodiments of any of the methods described herein, the bacterial strain is selected from the group consisting of: Roseburia hominus A2-183, Eubacterium eligens DSM 3376, and a combination thereof.

In some embodiments of any of the methods described herein, the bacterial strain improves intestinal barrier function of the subject.

In some embodiments of any of the methods described herein, the strain Roseburia hominus A2-183 has a 16S RNA gene that is at least 95% identical to SEQ ID NO:99. In some embodiments of any of the methods described herein, the strain Roseburia hominus A2-183 has a 16S RNA gene that is at least 95% identical to SEQ ID NO:100. In some embodiments of any of the methods described herein, the strain Roseburia hominus A2-183 has a 16S RNA gene that is at least 95% identical to SEQ ID NO:101. In some embodiments of any of the methods described herein, the strain Roseburia hominus A2-183 has a 16S RNA gene that is at least 95% identical to SEQ ID NO:102. In some embodiments of any of the methods described herein, the strain Roseburia hominus A2-183 has a 16S RNA gene that is at least 95% identical to SEQ ID NO:103. In some embodiments of any of the methods described herein, the strain Roseburia hominus A2-183 has a 16S RNA gene that is at least 95% identical to SEQ ID NO:104.

In some embodiments of any of the methods described herein, the strain Eubacterium eligens DSM 3376 has a 16S RNA gene that is at least 90% identical to SEQ ID NO:73. In some embodiments of any of the methods described herein, the strain Eubacterium eligens DSM 3376 has a 16S RNA gene that is at least 95% identical to SEQ ID NO:74. In some embodiments of any of the methods described herein, the strain Eubacterium eligens DSM 3376 has a 16S RNA gene that is at least 95% identical to SEQ ID NO:75. In some embodiments of any of the methods described herein, the strain Eubacterium eligens DSM 3376 has a 16S RNA gene that is at least 90% identical to SEQ ID NO:76. In some embodiments of any of the methods described herein, the strain Eubacterium eligens DSM 3376 has a 16S RNA gene that is at least 95% identical to SEQ ID NO:77. In some embodiments of any of the methods described herein, the strain Eubacterium eligens DSM 3376 has a 16S RNA gene that is at least 95% identical to SEQ ID NO:78. In some embodiments of any of the methods described herein, the strain Eubacterium eligens DSM 3376 has a 16S RNA gene that is at least 95% identical to SEQ ID NO:79.

In some embodiments of any of the methods described herein, the bacterial strain in the composition is viable. In some embodiments of any of the methods described herein, the bacterial strain is lyophilized.

In some embodiments of any of the methods described herein, the composition further comprises one or more cryopreservants.

In some embodiments of any of the methods described herein, the effective amount of the bacterial strain comprises at least about 1×10³ colony forming units (CFU) of the bacterial strain. In some embodiments of any of the methods described herein, the effective amount of the bacterial strain comprises about 1×10⁴ to about 1×10¹⁵ CFU of the bacterial strain. In some embodiments of any of the methods described herein, the effective amount of the bacterial strain comprises about 1×10⁶ to about 1×10¹⁰ CFU of the bacterial strain.

In some embodiments of any of the methods described herein, the bacterial strain in the composition is non-viable. In some embodiments of any of the methods described herein, the non-viable bacterial strain is heat-killed, irradiated, or lysed.

In some embodiments of any of the methods described herein, the method comprises administering the composition to the subject once, twice, or three times per day.

In some embodiments of any of the methods described herein, the composition is formulated for oral administration. In some embodiments of any of the methods described herein, the composition is formulated for rectal administration. In some embodiments of any of the methods described herein, the composition is formulated as a tablet, a capsule, a powder, or a liquid. In some embodiments of any of the methods described herein, the composition is formulated as a tablet. In some embodiments of any of the methods described herein, the tablet is coated. In some embodiments of any of the methods described herein, the coating comprises an enteric coating.

In some embodiments of any of the methods described herein, the method further comprises administering another treatment for ulcerative colitis and/or adjunct therapy to the subject. In some embodiments of any of the methods described herein, the composition comprising the bacterial strain and the treatment for ulcerative colitis and/or adjunct therapy are administered simultaneously. In some embodiments of any of the methods described herein, the composition comprising the bacterial strain and the treatment for ulcerative colitis and/or adjunct therapy are administered sequentially.

In some embodiments of any of the methods described herein, the treatment for ulcerative colitis and/or adjunct therapy comprises a probiotic.

In some embodiments of any of the methods described herein, the treatment for ulcerative colitis and/or adjunct therapy comprises a therapeutic agent. In some embodiments of any of the methods described herein, the therapeutic agent comprises an anti-inflammatory agent, an immunosuppressant, an antibiotic, an anti-diarrheal agent, a pain reliever, an iron supplement, or a combination thereof. In some embodiments of any of the methods described herein, the anti-inflammatory agent comprises sulfasalazine, mesalamine, balsalazide, olsalazine, prednisone, hydrocortisone, rednisone, budesonide or a combination thereof. In some embodiments of any of the methods described herein, the immunosuppressant comprises azathioprine, mercaptopurine, cyclosporine, infliximab, adalimumab, golimumab, vedolizumab, or a combination thereof.

In some embodiments of any of the methods described herein, the composition comprising the bacterial strain further comprises the therapeutic agent.

In some embodiments of any of the methods described herein, the subject is a human.

Also provided herein are methods for treating a subject in need thereof that include administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

In some embodiments of any of the methods described herein, the subject has ulcerative colitis.

Also provided herein are methods for treating ulcerative colitis in a subject that include administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

Also provided herein are methods for treating ulcerative colitis in a subject that include detecting a dysbiosis associated with ulcerative colitis in a sample from the subject; and administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

In some embodiments of any of the methods described herein, the sample is a biopsy sample. In some embodiments of any of the methods described herein, the sample is a colorectal biopsy sample. In some embodiments of any of the methods described herein, the sample is a fecal sample.

In some embodiments of any of the methods described herein, detecting the dysbiosis associated with ulcerative colitis comprises determining bacterial gene expression in the sample from the subject. In some embodiments of any of the methods described herein, detecting the dysbiosis associated with ulcerative colitis comprises determining bacterial composition in the sample from the subject. In some embodiments of any of the methods described herein, detecting the dysbiosis associated with ulcerative colitis comprises determining that Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased (e.g., enriched) in the sample from subject. In some embodiments of any of the methods described herein, detecting the dysbiosis associated with ulcerative colitis comprises determining that Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased in the sample from subject.

In some embodiments of any of the methods described herein, Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased in the gastrointestinal tract of the subject.

Also provided herein are methods for treating a subject in need thereof that include decreasing a population of an increased (e.g., enriched) bacterial strain in the subject, wherein the increased bacterial strain is selected from the group consisting of Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC45, and a combination thereof.

In some embodiments of any of the methods described herein, the subject has ulcerative colitis.

In some embodiments of any of the methods described herein, decreasing the population of an increased (e.g., enriched) bacterial strain comprises administering to the subject a bacteriophage. In some embodiments of any of the methods described herein, decreasing the population of an increased (e.g., enriched) bacterial strain comprises administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

In some embodiments of any of the methods described herein, the ulcerative colitis is distal colitis. In some embodiments of any of the methods described herein, the ulcerative colitis is extensive colitis. In some embodiments of any of the methods described herein, the ulcerative colitis is selected from the group consisting of: ulcerative proctitis, left-sided colitis, proctosigmoiditis, and pancolitis.

In some embodiments of any of the methods described herein, the bacterial strain comprises Bacillus sp. BT1B_CT2. In some embodiments of any of the methods described herein, the bacterial strain comprises Bacteroides coprocola M16. In some embodiments of any of the methods described herein, the bacterial strain comprises Bacteroides eggerthii CCUG 9559. In some embodiments of any of the methods described herein, the bacterial strain comprises Bacteroides vulgatus 8482. In some embodiments of any of the methods described herein, the bacterial strain comprises Butyricimonas virosa MT12. In some embodiments of any of the methods described herein, the bacterial strain comprises Coprococcus eutactus ATCC 27759. In some embodiments of any of the methods described herein, the bacterial strain comprises Desulfovibrio piger DSM 749. In some embodiments of any of the methods described herein, the bacterial strain comprises Dolosigranulum pigrum IFO 15550. In some embodiments of any of the methods described herein, the bacterial strain comprises Eubacterium biforme DSM 3989. In some embodiments of any of the methods described herein, the bacterial strain comprises Eubacterium eligens DSM 3376. In some embodiments of any of the methods described herein, the bacterial strain comprises Fusobacterium prausnitzii ATCC 27768. In some embodiments of any of the methods described herein, the bacterial strain comprises Howardella ureilytica GPC 589. In some embodiments of any of the methods described herein, the bacterial strain comprises Megasphaera elsdenii LC1. In some embodiments of any of the methods described herein, the bacterial strain comprises Parabacteroides johnsonii M-165. In some embodiments of any of the methods described herein, the bacterial strain comprises Phascolarctobacterium sp. YIT 12067. In some embodiments of any of the methods described herein, the bacterial strain comprises Roseburia hominus A2-183. In some embodiments of any of the methods described herein, the bacterial strain comprises Ruminococcus callidus VPI 57-31.

In some embodiments of any of the methods described herein, the bacterial strain is selected from the group consisting of: Roseburia hominus A2-183, Eubacterium eligens DSM 3376, and a combination thereof.

In some embodiments of any of the methods described herein, wherein the bacterial strain improves intestinal barrier function of the subject.

In some embodiments of any of the methods described herein, the strain Roseburia hominus A2-183 has a 16S RNA gene that is at least 95% identical to SEQ ID NO:99. In some embodiments of any of the methods described herein, the strain Roseburia hominus A2-183 has a 16S RNA gene that is at least 95% identical to SEQ ID NO:100. In some embodiments of any of the methods described herein, the strain Roseburia hominus A2-183 has a 16S RNA gene that is at least 95% identical to SEQ ID NO:101. In some embodiments of any of the methods described herein, the strain Roseburia hominus A2-183 has a 16S RNA gene that is at least 95% identical to SEQ ID NO:102. In some embodiments of any of the methods described herein, the strain Roseburia hominus A2-183 has a 16S RNA gene that is at least 95% identical to SEQ ID NO:103. In some embodiments of any of the methods described herein, the strain Roseburia hominus A2-183 has a 16S RNA gene that is at least 95% identical to SEQ ID NO:104.

In some embodiments of any of the methods described herein, the strain Eubacterium eligens DSM 3376 has a 16S RNA gene that is at least 90% identical to SEQ ID NO:73. In some embodiments of any of the methods described herein, the strain Eubacterium eligens DSM 3376 has a 16S RNA gene that is at least 95% identical to SEQ ID NO:74. In some embodiments of any of the methods described herein, the strain Eubacterium eligens DSM 3376 has a 16S RNA gene that is at least 95% identical to SEQ ID NO:75. In some embodiments of any of the methods described herein, the strain Eubacterium eligens DSM 3376 has a 16S RNA gene that is at least 90% identical to SEQ ID NO:76. In some embodiments of any of the methods described herein, the strain Eubacterium eligens DSM 3376 has a 16S RNA gene that is at least 95% identical to SEQ ID NO:77. In some embodiments of any of the methods described herein, the strain Eubacterium eligens DSM 3376 has a 16S RNA gene that is at least 95% identical to SEQ ID NO:78. In some embodiments of any of the methods described herein, the strain Eubacterium eligens DSM 3376 has a 16S RNA gene that is at least 95% identical to SEQ ID NO:79.

In some embodiments of any of the methods described herein, the bacterial strain in the composition is viable. In some embodiments of any of the methods described herein, the bacterial strain is lyophilized.

In some embodiments of any of the methods described herein, the composition further comprises one or more cryopreservants.

In some embodiments of any of the methods described herein, the effective amount of the bacterial strain comprises at least about 1×10³ colony forming units (CFU) of the bacterial strain. In some embodiments of any of the methods described herein, the effective amount of the bacterial strain comprises about 1×10⁴ to about 1×10¹⁵ CFU of the bacterial strain. In some embodiments of any of the methods described herein, the effective amount of the bacterial strain comprises about 1×10⁶ to about 1×10¹⁰ CFU of the bacterial strain.

In some embodiments of any of the methods described herein, the bacterial strain in the composition is non-viable. In some embodiments of any of the methods described herein, the non-viable bacterial strain is heat-killed, irradiated, or lysed.

In some embodiments of any of the methods described herein, the method comprises administering the composition to the subject once, twice, or three times per day.

In some embodiments of any of the methods described herein, the composition is formulated for oral administration. In some embodiments of any of the methods described herein, the composition is formulated for rectal administration. In some embodiments of any of the methods described herein, the composition is formulated as a tablet, a capsule, a powder, or a liquid. In some embodiments of any of the methods described herein, the composition is formulated as a tablet. In some embodiments of any of the methods described herein, the tablet is coated. In some embodiments of any of the methods described herein, the coating comprises an enteric coating.

In some embodiments of any of the methods described herein, the method further comprises administering another treatment for ulcerative colitis and/or adjunct therapy to the subject. In some embodiments of any of the methods described herein, the composition comprising the bacterial strain and the treatment for ulcerative colitis and/or adjunct therapy are administered simultaneously. In some embodiments of any of the methods described herein, the composition comprising the bacterial strain and the treatment for ulcerative colitis and/or adjunct therapy are administered sequentially. In some embodiments of any of the methods described herein, the treatment for ulcerative colitis and/or adjunct therapy comprises a probiotic.

In some embodiments of any of the methods described herein, the treatment for ulcerative colitis and/or adjunct therapy comprises a therapeutic agent. In some embodiments of any of the methods described herein, the therapeutic agent comprises an anti-inflammatory agent, an immunosuppressant, an antibiotic, an anti-diarrheal agent, a pain reliever, an iron supplement, or a combination thereof. In some embodiments of any of the methods described herein, the anti-inflammatory agent comprises sulfasalazine, mesalamine, balsalazide, olsalazine, prednisone, hydrocortisone, rednisone, budesonide or a combination thereof. In some embodiments of any of the methods described herein, the immunosuppressant comprises azathioprine, mercaptopurine, cyclosporine, infliximab, adalimumab, golimumab, vedolizumab, or a combination thereof.

In some embodiments of any of the methods described herein, the composition comprising the bacterial strain further comprises the therapeutic agent.

In some embodiments of any of the methods described herein, the subject is a human.

Provided herein are methods and compositions for treating a subject in need thereof. For example, methods provided herein can include administering to the subject a composition comprising an effective amount of a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof. In some embodiments, the bacterial strain is selected from the group consisting of Roseburia hominis A2-183, Eubacterium eligens DSM 3376, and a combination thereof. In some embodiments, the bacterial strain is Roseburia hominis A2-183. In some embodiments, the subject has ulcerative colitis.

Also, provided herein are methods for treating ulcerative colitis in a subject that include administering to the subject a composition comprising an effective amount of a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof. In some embodiments, the bacterial strain is selected from the group consisting of Roseburia hominis A2-183, Eubacterium eligens DSM 3376, and a combination thereof. In some embodiments, the bacterial strain is Roseburia hominis A2-183.

Also provided herein are methods for treating ulcerative colitis in a subject that include (a) detecting a dysbiosis associated with ulcerative colitis in a sample (e.g., a biopsy sample, a colorectal biopsy sample, or a fecal sample) from the subject; and (b) administering to the subject a composition comprising an effective amount of a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof. In some embodiments, the bacterial strain is selected from the group consisting of Roseburia hominis A2-183, Eubacterium eligens DSM 3376, and a combination thereof. In some embodiments, the bacterial strain is Roseburia hominis A2-183.

In some embodiments, detecting the dysbiosis associated with ulcerative colitis comprises determining bacterial gene expression in the sample from the subject. In some embodiments, detecting the dysbiosis associated with ulcerative colitis comprises determining bacterial composition in the sample from the subject. In some embodiments, detecting the dysbiosis associated with ulcerative colitis comprises determining that Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased (e.g., enriched) in the sample from subject. In some embodiments, detecting the dysbiosis associated with ulcerative colitis comprises determining that Acinetobacter johnsonii B8, Acinetobacter sp. LMG 995, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased (e.g., enriched) in the sample from subject.

In some embodiments, detecting the dysbiosis associated with ulcerative colitis comprises determining that Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased (e.g., decreased) in the sample from subject. In some embodiments, Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased (e.g., decreased) in the gastrointestinal tract of the subject.

Also provided herein are methods for treating a subject in need thereof (e.g., the subject has ulcerative colitis). The method can include decreasing a population of an increased (e.g., enriched) bacterial strain in the subject, wherein the increased (e.g., enriched) bacterial strain is selected from the group consisting of Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, and a combination thereof. In some embodiments, decreasing the population of an increased (e.g., enriched) bacterial strain can include administering to the subject a bacteriophage. In some embodiments, decreasing the population of an increased (e.g., enriched) bacterial strain can include administering to the subject a composition comprising an effective amount of a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof. In some embodiments, the bacterial strain is selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof. In some embodiments, the bacterial strain is selected from the group consisting of Roseburia hominis A2-183, Eubacterium eligens DSM 3376, and a combination thereof. In some embodiments, the bacterial strain is Roseburia hominis A2-183.

In some embodiments, the ulcerative colitis is distal colitis. In some embodiments, the ulcerative colitis is extensive colitis. In some embodiments, the ulcerative colitis is selected from the group consisting of ulcerative proctitis, left-sided colitis, proctosigmoiditis, and pancolitis.

In some embodiments, the bacterial strain comprises Bacillus sp. BT1B_CT2. In some embodiments, the bacterial strain comprises Bacteroides coprocola M16. In some embodiments, the bacterial strain comprises Bacteroides eggerthii CCUG 9559. In some embodiments, the bacterial strain comprises Bacteroides vulgatus 8482. In some embodiments, the bacterial strain comprises Butyricimonas virosa MT12. In some embodiments, the bacterial strain comprises Coprococcus eutactus ATCC 27759. In some embodiments, the bacterial strain comprises Desulfovibrio piger DSM 749. In some embodiments, the bacterial strain comprises Dolosigranulum pigrum IFO 15550. In some embodiments, the bacterial strain comprises Eubacterium biforme DSM 3989. In some embodiments, the bacterial strain comprises Eubacterium eligens DSM 3376. In some embodiments, the bacterial strain comprises Fusobacterium.prausnitzii ATCC 27768. In some embodiments, the bacterial strain comprises Howardella ureilytica GPC 589. In some embodiments, the bacterial strain comprises Megasphaera elsdenii LC1. In some embodiments, the bacterial strain comprises Parabacteroides johnsonii M-165. In some embodiments, the bacterial strain comprises Phascolarctobacterium sp. YIT 12067. In some embodiments, the bacterial strain comprises Roseburia hominis A2-183. In some embodiments, the bacterial strain comprises Ruminococcus callidus VPI 57-31.

In some embodiments, the bacterial strain is selected from the group consisting of: Roseburia hominus A2-183, Eubacterium eligens DSM 3376, and a combination thereof.

In some embodiments, the bacterial strain improves intestinal barrier function of the subject.

In some embodiments, strain Bacillus sp. BT1B_CT2 has a 16S RNA gene that is at least 95% identical to one or more (e.g., one, two, three, four, five, six, seven, eight, nine, ten, or all) of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12. In some embodiments, strain Bacteroides coprocola M16 has a 16S RNA gene that is at least 95% identical to one or more (e.g., one, two, three, four, five, six, seven, eight, nine, ten, eleven, or all) of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. In some embodiments, strain Bacteroides eggerthii CCUG 9559 has a 16S RNA gene that is at least 95% identical to one or more (e.g., one, two, three, four, five, or all) of SEQ ID NO:25, SEQ ID NO:26, SEQ ID NO:27, SEQ ID NO:28, SEQ ID NO:29, and SEQ ID NO:30. In some embodiments, strain Bacteroides vulgatus 8482 has a 16S RNA gene that is at least 95% identical to one or more (e.g., one, two, three, four, five, six, seven, eight, nine, ten, eleven, or all) of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42. In some embodiments, strain Butyricimonas virosa MT12 has a 16S RNA gene that is at least 95% identical to SEQ ID NO:43. In some embodiments, strain Coprococcus eutactus ATCC 27759 has a 16S RNA gene that is at least 95% identical to one or more (e.g., one, two, three, four, five, six, seven, eight, nine, ten, eleven, twelve, or all) of SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, and SEQ ID NO:56. In some embodiments, strain Desulfovibrio piger DSM 749 has a 16S RNA gene that is at least 95% identical to SEQ ID NO:57. In some embodiments, strain Dolosigranulum pigrum IFO 15550 has a 16S RNA gene that is at least 95% identical to one or more (e.g., one, two, three, four, five, six, seven, eight, nine, ten, or all) of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, and SEQ ID NO:68. In some embodiments, strain Eubacterium biforme DSM 3989 has a 16S RNA gene that is at least 95% identical to one or more (e.g., one, two, three, or all) of SEQ ID NO:69, SEQ ID NO:70, SEQ ID NO:71, and SEQ ID NO:72. In some embodiments, strain Eubacterium eligens DSM 3376 has a 16S RNA gene that is at least 95% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79. In some embodiments, strain Fusobacterium.prausnitzii ATCC 27768 has a 16S RNA gene that is at least 95% identical to SEQ ID NO:80. In some embodiments, strain Howardella ureilytica GPC 589 has a 16S RNA gene that is at least 95% identical to SEQ ID NO:81. In some embodiments, strain Megasphaera elsdenii LC1 has a 16S RNA gene that is at least 95% identical to one or more (e.g., one, two, three, four, five, six, seven, eight, or all) of SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90. In some embodiments, strain Parabacteroides johnsonii M-165 has a 16S RNA gene that is at least 95% identical to one or more (e.g., one, two, three, or all) of SEQ ID NO:91, SEQ ID NO:92, SEQ ID NO:93, and SEQ ID NO:94. In some embodiments, strain Phascolarctobacterium sp. YIT 12067 has a 16S RNA gene that is at least 95% identical to one or more (e.g., one, two, three, or all) of SEQ ID NO:95, SEQ ID NO:96, SEQ ID NO:97, and SEQ ID NO:98. In some embodiments, strain Roseburia hominus A2-183 has a 16S RNA gene that is at least 95% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104. In some embodiments, strain Ruminococcus callidus VPI 57-31 has a 16S RNA gene that is at least 95% identical to one or both of SEQ ID NO:105 and SEQ ID NO:106.

In some embodiments, strain Roseburia hominus A2-183 has a 16S RNA gene that is at least 90% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104. In some embodiments, strain Roseburia hominus A2-183 has a 16S RNA gene that is at least 94% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104. In some embodiments, strain Roseburia hominus A2-183 has a 16S RNA gene that is at least 99% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104.

In some embodiments, strain Eubacterium eligens DSM 3376 has a 16S RNA gene that is at least 90% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79. In some embodiments, strain Eubacterium eligens DSM 3376 has a 16S RNA gene that is at least 94% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79. In some embodiments, strain Eubacterium eligens DSM 3376 has a 16S RNA gene that is at least 99% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79.

In some embodiments, the bacterial strain in the composition is viable. In some embodiments, the bacterial strain is lyophilized. In some embodiments, the composition further comprises one or more cryopreservants.

In some embodiments, the effective amount of the bacterial strain comprises at least about 1×10³ colony forming units (CFU) of the bacterial strain. In some embodiments, the effective amount of the bacterial strain comprises about 1×10⁴ to about 1×10¹⁵ CFU of the bacterial strain. In some embodiments, the effective amount of the bacterial strain comprises about 1×10⁶ to about 1×10¹⁰ CFU of the bacterial strain.

In some embodiments, the bacterial strain in the composition is non-viable. In some embodiments, the non-viable bacterial strain is heat-killed, irradiated, or lysed.

In some embodiments, the method comprises administering the composition to the subject once, twice, or three times per day.

In some embodiments, the composition further includes one or more excipients. In some embodiments, the composition is formulated for oral administration. In some embodiments, the composition is formulated for rectal administration. In some embodiments, the composition is formulated as a tablet, a capsule, a powder, or a liquid. In some embodiments, the composition is formulated as a tablet. In some embodiments, the tablet is coated. In some embodiments, the coating comprises an enteric coating.

In some embodiments, methods described herein comprise administering a composition comprising a bacterial strain as described herein in combination with one or more treatments of ulcerative colitis and/or other adjunct therapies to the subject. In some embodiments, the treatment of ulcerative colitis and/or other adjunct therapy can be a therapeutic agent such as an anti-inflammatory agent, an immunosuppressant, an antibiotic, an anti-diarrheal agent, a pain reliever, an iron supplement, or a combination thereof. In some embodiments, the anti-inflammatory agent comprises sulfasalazine, mesalamine, balsalazide, olsalazine, prednisone, hydrocortisone, rednisone, budesonide or a combination thereof. In some embodiments, the immunosuppressant comprises azathioprine, mercaptopurine, cyclosporine, infliximab, adalimumab, golimumab, vedolizumab, or a combination thereof. In some embodiments, the treatment of ulcerative colitis and/or other adjunct therapy comprises a probiotic.

In some embodiments, the composition comprising the bacterial strain and the one or more treatments of ulcerative colitis and/or adjunct therapies are administered simultaneously (e.g., in the same composition or different compositions). In some embodiments, the composition comprising the bacterial strain and the one or more treatments of ulcerative colitis and/or other adjunct therapies are administered sequentially.

In any of the methods, the subject can be a human.

As used herein, the phrase an “effective amount” of a bacterial strain can refer to an amount of the bacterial strain sufficient enough to reduce or eliminate one or more symptoms of the disorder or in some cases, to effect a cure upon administration. Effective amounts of a bacterial strain will vary with the bacterial strain chosen, the particular condition or conditions being treated, the severity of the condition, the duration of the treatment, the specific components of the composition being used, and like factors. An “effective amount” can also refer to an amount of a combination of two or more bacterial strains or a combination of a bacterial strain and a therapeutic agent sufficient to reduce or eliminate one or more symptoms of the disorder or in some cases, to effect a cure upon administration. For example, an “effective amount” can refer to an amount of a combination of bacterial strains or a combination of a bacterial strain and another treatment (e.g., a therapeutic agent) when an additive or synergistic effect is observed with the combination compared to administration of the bacterial strain(s) and/or therapeutic agent(s) alone.

As used herein, “subject” or “patient” refers to any subject, particularly a mammalian subject such as a human, for whom diagnosis, prognosis, or therapy is desired.

As used herein, “treatment” or “treating” of a disease, disorder, or condition encompasses alleviation of at least one symptom thereof, a reduction in the severity thereof, or the delay or inhibition of the progression thereof. Treatment need not mean that the disease, disorder, or condition is totally cured. A useful composition herein needs only to reduce the severity of a disease, disorder, or condition, reduce the severity of one or more symptoms associated therewith, or improve a patient or subject's quality of life.

The term “preventing” as used herein means the prevention of the onset, recurrence, or spread, in whole or in part, of the disease or condition as described herein, or a symptom thereof.

The term “administration” or “administering” refers to a method of giving an amount of a bacterial strain, or a composition thereof, or other treatment of ulcerative colitis and/or adjunct therapy to a subject. The method of administration can vary depending on various factors, e.g., the components of the composition, the site of the disease, and the severity of the disease.

“Microbiome” refers to the collection of microorganisms and viruses and/or their genes from a given environment. For example, “microbiome” can refer to the collection of the microorganisms and viruses and/or their genes from the gastrointestinal tract of humans. “Microbiota” refers to the microorganisms in a specific environment.

“Dysbiosis” refers to a state of the microbiota or microbiome of the gut or other body area (e.g., mucosal or skin surfaces or any other microbiota niche) of a subject (i.e., the host) in which the diversity and/or function of the ecological network is disrupted, e.g., as compared to the state of the microbiota or microbiome of the gut or other body area in a control population (e.g., reference population). A control population can include individuals that meet one or more qualifications such as individuals that have not been diagnosed with a disease (e.g., the same disease as the subject); individuals that do not have a known genetic predisposition to a disease (e.g., the same disease as the subject); or individuals that do not have a known environmental predisposition to a disease (e.g., the same disease as the subject); or individuals that do not have a known predisposition that would prevent treatment of and/or recovery from a disease (e.g., the same disease as the subject). In some embodiments, the individuals in the control population meet one of the above control population qualifications. In some embodiments, the individuals in the control population meet two of the above control population qualifications. In some embodiments, the individuals in the control population meet three of the above control population qualifications. In some embodiments, the individuals in the control population meet four of the above control population qualifications. In some embodiments, the control population is homogenous with respect to at least one of the qualifications. Any disruption in the microbiota or microbiome of a subject (i.e., host) compared to the microbiota or microbiome of a control population can be considered a dysbiosis, even if such dysbiosis does not result in a detectable decrease in health of the subject. Dysbiosis in a subject may be unhealthy for the subject (e.g., result in a diseased state in the subject), it may be unhealthy for the subject under only certain conditions (e.g., result in diseased state under only certain conditions), or it may prevent the subject from becoming healthier (e.g., may prevent a subject from responding to treatment or recovering from a disease or disorder). Dysbiosis may be due to a decrease in diversity of the microbiota population composition (e.g., a depletion of one or more bacterial strains, an overgrowth of one or more bacterial strains, or a combination thereof), the overgrowth of one or more population of pathogens (e.g., a population of pathogenic bacteria) or pathobionts, the presence of and/or overgrowth of a symbiotic organism able to cause disease only when certain genetic and/or environmental conditions are present in a subject, or a shift to an ecological network that no longer provides a beneficial function to the host and therefore no longer promotes health.

As used herein the terms “microorganism” or “microbe” should be taken broadly. These terms are used interchangeably and include, but are not limited to, the two prokaryotic domains, Bacteria and Archaea, as well as eukaryotic fungi and protists. In some embodiments, the disclosure refers to a “bacterium” or a “microbe.” This characterization can refer to not only the identified taxonomic bacterial genera of the microbe, but also the identified taxonomic species, as well as the bacterial strains. A “strain” can include descendants of a single isolation in pure culture that is usually made up of a succession of cultures ultimately derived from an initial single colony. In some embodiments, a strain includes an isolate or a group of isolates that can be distinguished from other isolates of the same genus and species by phenotypic characteristics, genotypic characteristics, or both.

The term “relative abundance” as used herein, is the number or percentage of a microbe present in the gastrointestinal tract or any other microbiota niche of a subject, such as the ocular, placental, lung, cutaneous, urogenital, or oral microbiota niches, relative to the number or percentage of total microbes present in the gastrointestinal tract or the other microbiota niche of the subject. The relative abundance may also be determined for particular types of microbes such as bacteria, fungi, viruses, and/or protozoa, relative to the total number or percentage of bacteria, fungi, viruses, and/or protozoa present. Relative abundance can be determined by a number of methods readily known to the ordinarily skilled artisan, including, but not limited to, array or microarray hybridization, sequencing, quantitative PCR, and culturing and performance of colony forming unit (cfu, CFU) assays or plaque forming unit (pfu, PFU) assays performed on a sample from the gastrointestinal tract or other microbiota niche.

As used herein, terms such as “isolate” and “isolated” in reference to a microbe, are intended to mean that a microbe has been separated from at least one of the materials with which it is associated in a particular environment (for example, gastrointestinal fluid, gastrointestinal tissue, human digestive fluid, human digestive tissue, etc.). Accordingly, an “isolated microbe” does not exist in its naturally occurring environment. In some embodiments, an isolated microbe, e.g., a bacterial strain, may exist as, for example, a biologically pure culture, or as spores (or other forms of the bacterial strain) in association with a pharmaceutically acceptable excipient suitable for human administration. In some embodiments, more than one microbe can be isolated. For example, “isolated microbes” can refer to a mixture of two or more microbes that have been separated from at least one of the materials with which they are associated in a particular environment.

In some embodiments, the isolated microbes exist as isolated and biologically pure cultures. As used herein, the term “biologically pure” refers to a composition comprising a species or strains of a microbe, wherein the composition is substantially free from the material from which the microbe was isolated or produced and from other microbes (e.g., other species or strains and other microbes of a different taxonomic classification). In some embodiments, “biologically pure” can refer to a composition that comprises a strain of a bacterial strain that is substantially free from the material from which the bacterial strain was isolated or produced and from other microbes, e.g., other strains of the same bacterial strain, other species of the same bacteria, and other bacteria and/or microbes of a different taxonomic classification). It will be appreciated by one of skill in the art, that an isolated and biologically pure culture of a particular microbe, denotes that said culture is substantially free (within scientific reason) of other living organisms and contains only the individual microbe in question. As used herein, “substantially free” means that a composition comprising a species or strain of a microbe is at least about 80%, about 85%, about 90%, about 91%, about 92%, about 93%, about 94%, about 95%, about 96%, about 97%, about 98%, about 99%, or more than about 99% free of the material from which the microbe was isolated or produced and from other microbes. In some embodiments, a biologically pure composition contains no other bacterial strain in quantities that can be detected by typical bacteriological techniques.

As used herein, the term “mutation” includes a natural or induced mutation comprising at least a single base or amino acid alteration in a DNA or protein sequence. For example, a mutation can include a base substitution, a deletion, an insertion, a transversion, or any other modification known to those skilled in the art, including a genetic modification introduced into a parent nucleotide or an amino acid sequence.

As used herein, “probiotic” refers to a substantially pure microbe (i.e., a single isolate) or a mixture of microbes, and may also include any additional components that can be administered to a subject (e.g., a human), for restoring or altering the microbiota or microbiome in the subject. In some embodiments, a probiotic or microbial inoculant composition can be administered with an agent to allow the microbe(s) to survive the environment of the gastrointestinal tract, i.e., to resist low pH and/or to grow in the gastrointestinal environment. In some embodiments, a composition as described herein includes a probiotic.

As used herein, “prebiotic” refers to an agent that increases the number and/or activity of one or more microbes. Such microbes can include microbes for restoring or altering the microbiota or microbiome of a subject. Non-limiting examples of a prebiotic include a fructooligosaccharide (e.g., oligofructose, inulin, or an inulin-type fructan), a galactooligosaccharide, an amino acid, an alcohol. See, for example, Ramirez-Farias et al. (2008. Br. J Nutr. 4:1-10) and Pool-Zobel and Sauer (2007. J Nutr. 137:2580-2584).

As used herein, a “live biotherapeutic product” or “LBP” refers to a biological product that: 1) contains live organisms, such as bacteria, and 2) is applicable to the prevention, treatment, and/or cure of a disease or condition of a subject.

A “combination” of two or more bacteria, e.g., bacterial strains, can refer to the physical co-existence of the bacteria, either in the same material or product. In some embodiments, a combination of two or more bacteria can include the temporal co-administration or co-localization of the two or more bacteria.

The terms “percent identity” or “identity” in the context of two or more nucleic acids or polypeptides, refers to the measurement of the similarity between the two or more sequences. The percent identity can be measured by any method known to one of skill in the art including using a sequence comparison software, an algorithm, and by visual inspection.

In general, the percent identity for two or more sequences (e.g., a nucleic acid or amino acid sequence), also referred to as the “percent sequence identity”, is calculated by determining the number of matched positions in the aligned nucleic acid or amino acid sequences, dividing the number of matched positions by the total number of aligned nucleotides or amino acids, respectively, and multiplying by 100. A matched position refers to a position in which identical nucleotides or amino acids occur at the same position in the aligned sequences. As an example, the total number of aligned nucleotides can refer to the minimum number of the 16S rRNA gene nucleotides that are necessary to align the second sequence, and does not include alignment (e.g., forced alignment) with non-16S rRNA gene sequences. The total number of aligned nucleotides may correspond to the entire 16S rRNA gene sequence or may correspond to fragments of the full-length 16S rRNA gene sequence.

Sequences can be aligned using an algorithm, for example, the algorithm as described by Altschul et al. (Nucleic Acids Res, 25:3389-3402, 1997) and incorporated into BLAST (basic local alignment search tool) programs, which are available at ncbi.nlm.nih.gov. BLAST searches or alignments can be performed to determine percent sequence identity between a 16S rRNA gene nucleic acid and any other sequence or portion thereof using the Altschul et al. algorithm. BLASTN can be used to align and compare the identity between nucleic acid sequences, while BLASTP can be used to align and compare the identity between amino acid sequences. When utilizing a BLAST program to calculate the percent identity between a 16S rRNA gene sequence and another sequence, the default parameters of the program are used.

Generally, a bacterial strain genomic sequence will contain multiple copies of 16S rRNA sequences. The 16S rRNA sequences can be used for making distinctions between species and strains. For example, if one or more of the 16S rRNA sequences shares less than 97% sequence identity from a reference sequence, then the two organisms from which the sequences were obtained can be of different species or strains.

The term “combination therapy” as used herein refers to a dosing regimen of one or more bacterial strains and one or more other treatments of ulcerative colitis and/or adjunct therapies, wherein the bacterial strain and other treatment (e.g., a therapeutic agent) are administered together or separately in a manner prescribed by a medical care taker or according to a regulatory agency. As can be appreciated in the art, a combination therapy can be administered to a patient for a period of time. In some embodiments, the period of time occurs following the administration of one or more of: a different bacterial strain, a different treatment/therapeutic agent, and a different combination of treatments/therapeutic agents to the subject. In some embodiments, the period of time occurs before the administration of one or more of: a different bacterial strain, a different treatment/therapeutic agent, and a different combination of therapeutic treatments/agents to the subject.

The term “fixed combination” means that one or more bacterial strains as described herein, or a composition thereof, and at least one other treatment and/or adjunct therapy (e.g., a prebiotic, a probiotic, an immunosuppressant, an anti-inflammatory agent, an antibiotic, an anti-diarrheal agent, a pain reliever, an iron supplement, or a combination thereof), are both administered to a subject simultaneously in the form of a single composition or dosage.

The term “non-fixed combination” means that one or more bacterial strains as described herein, or a composition thereof, and at least one other treatment or adjunct therapy (e.g., a prebiotic, a probiotic, an immunosuppressant, an anti-inflammatory agent, an antibiotic, an anti-diarrheal agent, a pain reliever, an iron supplement, or a combination thereof) are formulated as separate compositions or dosages such that they may be administered to a subject simultaneously or sequentially with variable intervening time limits. These also apply to cocktail therapies, e.g., the administration of three or more therapeutic agents.

Reference to the term “about” has its usual meaning in the context of compositions to allow for reasonable variations in amounts that can achieve the same effect and also refers herein to a value of plus or minus 10% of the provided value. For example, “about 20” means or includes amounts from 18 to and including 22.

Unless otherwise required by context, singular terms shall include pluralities and plural terms shall include the singular. As used herein, the singular form “a”, “an”, and “the” include plural references unless indicated otherwise. For example, “an” excipient includes one or more excipients. It is understood that aspects and variations of the invention described herein include “consisting of” and/or “consisting essentially of” aspects and variations.

The details of one or more embodiments of the invention are set forth in the accompanying drawings and the description below. Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1 is an exemplary schematic of the analysis.

FIG. 2 has plots showing the tallies of strains detected (after prevalence filtering). The top panel tallies the strains detected in increasing number of datasets. The middle panel shows the number of strains that were significantly differentially abundant in 1 or more datasets. The bottom panel shows the number of strains that exhibit concordance in direction of the log 2 fold change, in increasing proportions of datasets in which they were detected. Only strains detected in at least two datasets are included.

FIG. 3 is a plot showing that significant findings from isolated dataset analysis rarely exhibits concordance in the direction of shifts across multiple datasets. Each row represents a strain identified as significantly differentially abundant by at least one isolated analysis. Asterisk denotes dataset(s) where significant changes of a strain was observed. Cell color indicates direction of the log 2 fold change: decreased (e.g., decreased) (green) or increased (e.g., enriched) (pink) in dysbiosis and not detected (white). Cells are shaded dark if the direction of log 2 fold change is concordant across all datasets a strain was detected in, i.e., the strain is associated with homeostasis or dysbiosis only. See FIG. 9 for details on contrasts analyzed within each cohort of subjects.

FIG. 4 is a plot showing the distribution of effect sizes per dataset. 16S-Hyb refers to profiling of the 16S rRNA gene on the PhyloChip; 16S-NGS and 16S-Sanger refer to sequencing of the 16S rRNA gene via next generation sequencing (NGS) and Sanger sequencing, respectively.

FIG. 5 is a plot showing strains (dots) significantly differentially abundant in eubiotic or dysbiotic state by isolated-dataset analysis (grey squares) or MTMA (grey spheres) in atopic dermatitis. Strains (dots) are sized by the number of datasets in which they were detected and colored as follows: significant by isolated analysis only (dark green); MTMA only (purple); or both (blue). Solid lines connect MTMA results to strains and dashed lines connect isolated analysis results to strains. Thick and thin lines indicate significant and non-significant findings, respectively. Red and green lines indicate enrichment and reduction in dysbiotic state, respectively. Annotation for strains described here are provided in FIG. 10.

FIG. 6 is a forest plot demonstrating distribution of log 2 fold changes and 95% confidence intervals for strains that were identified as significantly differentially abundant by MTMA. Circles and triangles indicate log 2 fold change estimated by isolated analysis and MTMA, respectively. Error bars in the forest plots correspond to the 95% confidence interval. Green and blue indicate significant and nonsignificant findings, respectively, and grey indicates cases where an adjusted p-value could not be imputed by the statistical test.

FIG. 7 is a plot of MTMA-derived adjusted p-values and log 2 fold changes. Data points are shaded according to the proportion of datasets in which the strain was detected. Significantly dysbiosis-associated strains plot in the upper left quadrant, whereas homeostasis-associated strains plot in the upper right quadrant. FIG. 10 provides strain names for strain identifiers indicated in the plot.

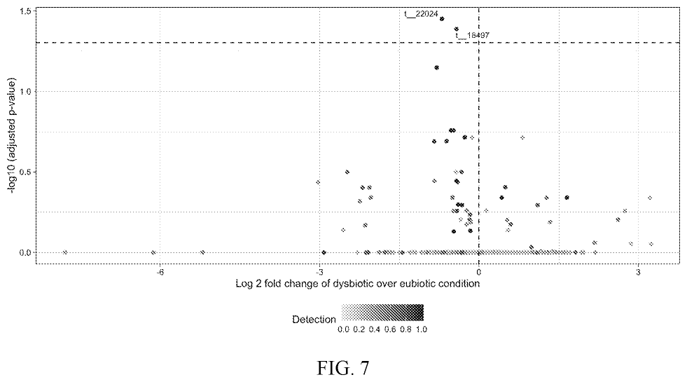

FIG. 8 has plots showing the 4KDa-FITC and colon weight/length for mice administered Roseburia hominus A2-183 and Eubacterium eligens DSM 3376 cocktail. Mice were administered 2.5% DSS in the drinking water and were concurrently treated with vehicle, or Roseburia hominus A2-183 (2.2×10⁸ CFU) and Eubacterium eligens DSM 3376 (4.7×10⁷ CFU) cocktail twice a day by oral gavage (n=10 per group). On day six, a, translocation of 4KDa-FITC-dextran into serum was measured following oral gavage and b, colon weight to length ratios were calculated. Statistical analyses were performed via a one-way ANOVA compared to DSS+vehicle using a Dunnett's multiple comparison test, *=p<0.05. Graphs depict mean±SEM and summarize the data of an individual experiment.

FIG. 9 is a table showing details on contrasts analyzed within each cohort of subjects.

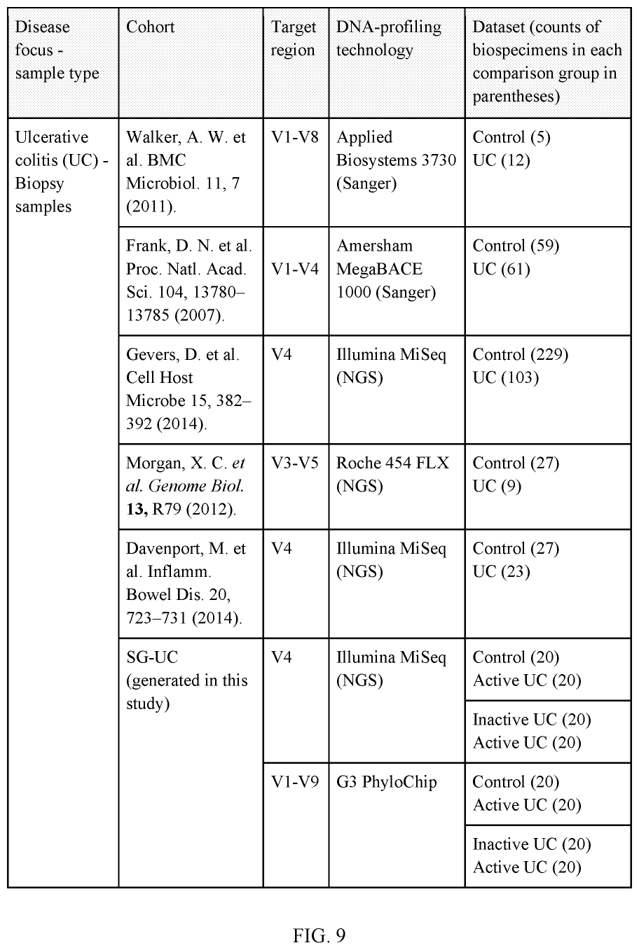

FIG. 10 is a table showing the association between the strain identifiers and strain names.

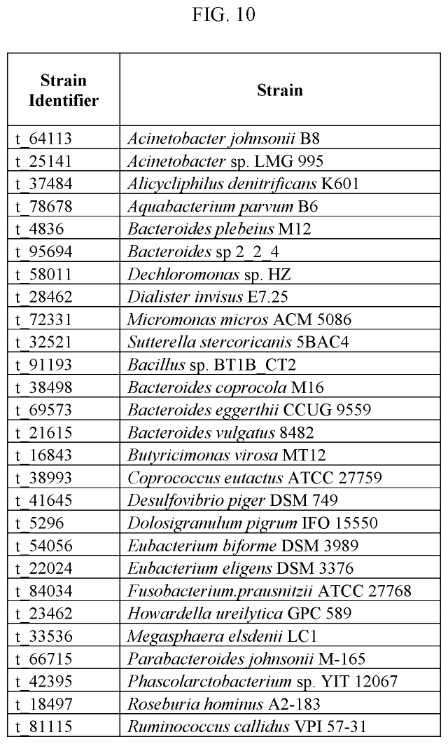

### DETAILED DESCRIPTION

This document provides compositions and methods for treating subjects in need thereof (e.g., subjects having ulcerative colitis) using one or more bacterial strains. Inflammation involves the activation of the immune system in response to harmful stimuli, such as a pathogen, an infection, an irritant, or damage to cells. Inflammatory disorders can involve the immune system attacking the body's own cells or tissues, which can lead to abnormal inflammation. Ulcerative colitis can cause long-lasting inflammation and sores (ulcers), in the innermost lining of the large intestine (colon) and rectum. Ulcerative colitis typically presents with shallow, continuous inflammation. This inflammation generally extends from the rectum and can include part of or the entire colon, and classification of ulcerative colitis is typically according to its location. For example, ulcerative proctitis can refer to ulcerative colitis in which inflammation is confined to the area closest to the anus; proctosigmoiditis can refer to ulcerative colitis in which inflammation involves the rectum and sigmoid colon (lower end of the colon); left-sided colitis (e.g., distal colitis) can refer to ulcerative colitis in which inflammation extends from the rectum up through the sigmoid and descending colon; pancolitis can refer to ulcerative colitis that affects the entire colon; and acute severe ulcerative colitis refers to ulcerative colitis that affects the entire colon and causes severe pain, profuse diarrhea, bleeding, fever and inability to eat. Subjects with inflammation limited to part of the colon (e.g., proctitis) can have mild but frequently recurrent symptoms, whereas subjects with extensive disease (e.g., pancolitis) more commonly have severe symptoms. See, for example, Botoman et al., Am. Fam. Physician, Vol. 57(1):57-68, 1998.

In some embodiments, methods for treating a subject in need thereof are provided herein. In some embodiments, one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are decreased (e.g., decreased) in a sample (e.g., fecal sample or a biopsy sample such as an intestinal biopsy sample or a colorectal biopsy sample) from the subject in need thereof. For example, one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are decreased (e.g., reduced) in the sample (e.g., fecal sample or a biopsy sample such as an intestinal biopsy sample or a colorectal biopsy sample) from the subject in need thereof compared to a control sample (e.g., a reference sample). Determining that one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are decreased (e.g., reduced) in the sample from the subject in need thereof can comprise sequencing one or more nucleic acids from the bacteria. In some embodiments, the subject in need thereof has been diagnosed with ulcerative colitis.

The methods provided herein can include administering to the subject a composition that includes an effective amount of a bacterial strain. In some embodiments, the bacterial strain can be selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof (e.g., any two, any three, any four, any five, any six, any seven, any eight, any nine, any ten, any eleven, any twelve, any thirteen, any fourteen, any fifteen, any sixteen, or all seventeen of the bacterial strains). In some embodiments, the bacterial strain can be selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof (e.g., any two, any three, any four, any five, any six, any seven, any eight, any nine, any ten, any eleven, any twelve, any thirteen, any fourteen, any fifteen, or all sixteen of the bacterial strains). In some embodiments, the bacterial strain can be selected from the group consisting of Roseburia hominis A2-183, Eubacterium eligens DSM 3376, and a combination thereof. In some embodiments, the bacterial strain is Roseburia hominis A2-183.

In some embodiments, the bacterial strain in the composition comprises Bacillus sp. BT1B_CT2. In some embodiments, Bacillus sp. BT1B_CT2 included in a composition provided herein has a 16S RNA gene that is at least 90% identical to one or more (e.g., one, two, three, four, five, six, seven, eight, nine, ten, or all) of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, or all) of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

In some embodiments, the bacterial strain in the composition comprises Bacteroides coprocola M16. A complete genomic sequence for Bacteroides coprocola M16 is available in the GenBank database as, e.g., Accession No. GCF_000154845. In some embodiments, the Bacteroides coprocola M16 included in a composition provided herein can have a genomic sequence with at least about 95% sequence identity to the genomic sequence published as GCF_000154845. For example, Bacteroides coprocola M16 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000154845. In some embodiments, Bacteroides coprocola M16 included in a composition provided herein has a 16S RNA gene that is at least 90% identical to one or more (e.g., one, two, three, four, five, six, seven, eight, nine, ten, eleven, or all) of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven, or all) of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, and SEQ ID NO:24.

In some embodiments, the bacterial strain in the composition comprises Bacteroides eggerthii CCUG 9559. A complete genomic sequence for Bacteroides eggerthii CCUG 9559 is available in the GenBank database as, e.g., Accession No. GCF_000155815. In some embodiments, the Bacteroides eggerthii CCUG 9559 included in a composition provided herein can have a genomic sequence with at least about 95% sequence identity to the genomic sequence published as GCF_000155815. For example, Bacteroides eggerthii CCUG 9559 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000155815. In some embodiments, Bacteroides eggerthii CCUG 9559 included in a composition provided herein has a 16S RNA gene that is at least 90% identical to one or more (e.g., one, two, three, four, five, or all) of SEQ ID NO:25, SEQ ID NO:26, SEQ ID NO:27, SEQ ID NO:28, SEQ ID NO:29, and SEQ ID NO:30. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:25, SEQ ID NO:26, SEQ ID NO:27, SEQ ID NO:28, SEQ ID NO:29, and SEQ ID NO:30.

In some embodiments, the bacterial strain in the composition comprises Bacteroides vulgatus 8482. A complete genomic sequence for Bacteroides vulgatus 8482 is available in the GenBank database as, e.g., Accession No. GCF_000012825. In some embodiments, the Bacteroides vulgatus 8482 included in a composition provided herein can have a genomic sequence with at least about 95% sequence identity to the genomic sequence published as GCF_000012825. For example, Bacteroides vulgatus 8482 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000012825. In some embodiments, Bacteroides vulgatus 8482 included in a composition provided herein has a 16S RNA gene that is at least 90% identical to one or more (e.g., one, two, three, four, five, six, seven, eight, nine, ten, eleven, or all) of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven, or all) of SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, and SEQ ID NO:42.

In some embodiments, the bacterial strain in the composition comprises Butyricimonas virosa MT12. In some embodiments, Butyricimonas virosa MT12 included in a composition provided herein has a 16S RNA gene that is at least 90% identical to SEQ ID NO:43. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:43.

In some embodiments, the bacterial strain in the composition comprises Coprococcus eutactus ATCC 27759. A complete genomic sequence for Coprococcus eutactus ATCC 27759 is available in the GenBank database as, e.g., Accession No. GCF_000154425. In some embodiments, the Coprococcus eutactus ATCC 27759 included in a composition provided herein can have a genomic sequence with at least about 95% sequence identity to the genomic sequence published as GCF_000154425. For example, Coprococcus eutactus ATCC 27759 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000154425. In some embodiments, Coprococcus eutactus ATCC 27759 included in a composition provided herein has a 16S RNA gene that is at least 90% identical to one or more (e.g., one, two, three, four, five, six, seven, eight, nine, ten, eleven, twelve, or all) of SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, and SEQ ID NO:56. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, eleven, twelve, or all) of SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, and SEQ ID NO:56.

In some embodiments, the bacterial strain in the composition comprises Desulfovibrio piger DSM 749. A complete genomic sequence for Desulfovibrio piger DSM 749 is available in the GenBank database as, e.g., Accession No. GCF_000156375. In some embodiments, the Desulfovibrio piger DSM 749 included in a composition provided herein can have a genomic sequence with at least about 95% sequence identity to the genomic sequence published as GCF_000156375. For example, Desulfovibrio piger DSM 749 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156375. In some embodiments, Desulfovibrio piger DSM 749 included in a composition provided herein has a 16S RNA gene that is at least 90% identical to SEQ ID NO:57. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:57.

In some embodiments, the bacterial strain in the composition comprises Dolosigranulum pigrum IFO 15550. A complete genomic sequence for Dolosigranulum pigrum IFO 15550 is available in the GenBank database as, e.g., Accession No. GCF_000245815. In some embodiments, the Dolosigranulum pigrum IFO 15550 included in a composition provided herein can have a genomic sequence with at least about 95% sequence identity to the genomic sequence published as GCF_000245815. For example, Dolosigranulum pigrum IFO 15550 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000245815. In some embodiments, Dolosigranulum pigrum IFO 15550 included in a composition provided herein has a 16S RNA gene that is at least 90% identical to one or more (e.g., one, two, three, four, five, six, seven, eight, nine, ten, or all) of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, and SEQ ID NO:68. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, or all) of SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, and SEQ ID NO:68.

In some embodiments, the bacterial strain in the composition comprises Eubacterium biforme DSM 3989. A complete genomic sequence for Eubacterium biforme DSM 3989 is available in the GenBank database as, e.g., Accession No. GCF_000156655. In some embodiments, the Eubacterium biforme DSM 3989 included in a composition provided herein can have a genomic sequence with at least about 95% sequence identity to the genomic sequence published as GCF_000156655. For example, Eubacterium biforme DSM 3989 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156655. In some embodiments, Eubacterium biforme DSM 3989 included in a composition provided herein has a 16S RNA gene that is at least 90% identical to one or more (e.g., one, two, three, or all) of SEQ ID NO:69, SEQ ID NO:70, SEQ ID NO:71, and SEQ ID NO:72. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:69, SEQ ID NO:70, SEQ ID NO:71, and SEQ ID NO:72.

In some embodiments, the bacterial strain in the composition comprises Eubacterium eligens DSM 3376. A complete genomic sequence for Eubacterium eligens DSM 3376 is available in the GenBank database as, e.g., Accession No. GCF_000146185. In some embodiments, the Eubacterium eligens DSM 3376 included in a composition provided herein can have a genomic sequence with at least about 95% sequence identity to the genomic sequence published as GCF_000146185. For example, Eubacterium eligens DSM 3376 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000146185. In some embodiments, Eubacterium eligens DSM 3376 included in a composition provided herein has a 16S RNA gene that is at least 90% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:73, SEQ ID NO:74, SEQ ID NO:75, SEQ ID NO:76, SEQ ID NO:77, SEQ ID NO:78, and SEQ ID NO:79.

In some embodiments, the bacterial strain in the composition comprises Fusobacterium.prausnitzii ATCC 27768. In some embodiments, Fusobacterium.prausnitzii ATCC 27768 included in a composition provided herein has a 16S RNA gene that is at least 90% identical to SEQ ID NO:80. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:80.

In some embodiments, the bacterial strain in the composition comprises Howardella ureilytica GPC 589. In some embodiments, Howardella ureilytica GPC 589 included in a composition provided herein has a 16S RNA gene that is at least 90% identical to SEQ ID NO:81. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:81.

In some embodiments, the bacterial strain in the composition comprises Megasphaera elsdenii LC1. A complete genomic sequence for Megasphaera elsdenii LC1 is available in the GenBank database as, e.g., Accession No. GCF_000283495. In some embodiments, the Megasphaera elsdenii LC1 included in a composition provided herein can have a genomic sequence with at least about 95% sequence identity to the genomic sequence published as GCF_000283495. For example, Megasphaera elsdenii LC1 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000283495. In some embodiments, Megasphaera elsdenii LC1 included in a composition provided herein has a 16S RNA gene that is at least 90% identical to one or more (e.g., one, two, three, four, five, six, seven, eight, or all) of SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, or all) of SEQ ID NO:82, SEQ ID NO:83, SEQ ID NO:84, SEQ ID NO:85, SEQ ID NO:86, SEQ ID NO:87, SEQ ID NO:88, SEQ ID NO:89, and SEQ ID NO:90.

In some embodiments, the bacterial strain in the composition comprises Parabacteroides johnsonii M-165. A complete genomic sequence for Parabacteroides johnsonii M-165 is available in the GenBank database as, e.g., Accession No. GCF_000156495. In some embodiments, the Parabacteroides johnsonii M-165 included in a composition provided herein can have a genomic sequence with at least about 95% sequence identity to the genomic sequence published as GCF_000156495. For example, Parabacteroides johnsonii M-165 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000156495. In some embodiments, Parabacteroides johnsonii M-165 included in a composition provided herein has a 16S RNA gene that is at least 90% identical to one or more (e.g., one, two, three, or all) of SEQ ID NO:91, SEQ ID NO:92, SEQ ID NO:93, and SEQ ID NO:94. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:91, SEQ ID NO:92, SEQ ID NO:93, and SEQ ID NO:94.

In some embodiments, the bacterial strain in the composition comprises Phascolarctobacterium sp. YIT 12067. A complete genomic sequence for Phascolarctobacterium sp. YIT 12067 is available in the GenBank database as, e.g., Accession No. GCF_000188175_. In some embodiments, the Phascolarctobacterium sp. YIT 12067 included in a composition provided herein can have a genomic sequence with at least about 95% sequence identity to the genomic sequence published as GCF_000188175. For example, Phascolarctobacterium sp. YIT 12067 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000188175. In some embodiments, Phascolarctobacterium sp. YIT 12067 included in a composition provided herein has a 16S RNA gene that is at least 90% identical to one or more (e.g., one, two, three, or all) of SEQ ID NO:95, SEQ ID NO:96, SEQ ID NO:97, and SEQ ID NO:98. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, or all) of SEQ ID NO:95, SEQ ID NO:96, SEQ ID NO:97, and SEQ ID NO:98.

In some embodiments, the bacterial strain in the composition comprises Roseburia hominis A2-183. A complete genomic sequence for Roseburia hominis A2-183 is available in the GenBank database as, e.g., Accession No. GCF_000225345. In some embodiments, the Roseburia hominis A2-183 included in a composition provided herein can have a genomic sequence with at least about 95% sequence identity to the genomic sequence published as GCF_000225345. For example, Roseburia hominis A2-183 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000225345. In some embodiments, Roseburia hominis A2-183 included in a composition provided herein has a 16S RNA gene that is at least 90% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:99, SEQ ID NO:100, SEQ ID NO:101, SEQ ID NO:102, SEQ ID NO:103, and SEQ ID NO:104.

In some embodiments, the bacterial strain in the composition comprises Ruminococcus callidus VPI 57-31. A complete genomic sequence for Ruminococcus callidus VPI 57-31 is available in the GenBank database as, e.g., Accession No. GCF_000468015. In some embodiments, the Ruminococcus callidus VPI 57-31 included in a composition provided herein can have a genomic sequence with at least about 95% sequence identity to the genomic sequence published as GCF_000468015. For example, Ruminococcus callidus VPI 57-31 included in a composition provided herein can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000468015. In some embodiments, Ruminococcus callidus VPI 57-31 included in a composition provided herein has a 16S RNA gene that is at least 90% identical to one or both of SEQ ID NO:105 and SEQ ID NO:106. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:105 and SEQ ID NO:106.

In some embodiments, the composition can include two or more bacterial strains selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31. For example, the composition can include three or more, four or more, five or more, six or more, seven or more, eight or more, nine or more, ten or more, eleven or more, twelve or more, thirteen or more, fourteen or more, fifteen or more, sixteen or more, or all seventeen bacterial strains selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31. Identifying characteristics of each strain are described above.

In some embodiments, the composition can include two or more bacterial strains selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31. For example, the composition can include three or more, four or more, five or more, six or more, seven or more, eight or more, nine or more, ten or more, eleven or more, twelve or more, thirteen or more, fourteen or more, fifteen or more, or all sixteen bacterial strains selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31. Identifying characteristics of each strain are described above.

In some embodiments, a method can include detecting, in a sample from the subject, a dysbiosis associated with ulcerative colitis, e.g., before administering to the subject an effective amount of a bacterial strain or a composition containing the bacterial strain. The sample can be a biopsy sample such as an intestinal biopsy sample or a colorectal biopsy sample. In some embodiments, the sample is a fecal sample.

In some embodiments, detecting the dysbiosis associated with ulcerative colitis can include determining bacterial gene expression in the sample from the subject (e.g., fecal sample or a biopsy sample such as an intestinal biopsy sample or a colorectal biopsy sample). For example, the bacterial gene expression can be determined in the sample from the subject e.g., before administering to the subject an effective amount of a bacterial strain or a composition containing the bacterial strain and/or after administering to the subject an effective amount of a bacterial strain or a composition containing the bacterial strain. Determining the bacterial gene expression can include performing, for example, RNAseq and/or RT-qPCR. In some embodiments, detecting the dysbiosis associated with ulcerative colitis comprises determining bacterial composition in the sample from the subject (e.g., fecal sample or a biopsy sample such as an intestinal biopsy sample or a colorectal biopsy sample). For example, the bacterial composition can be determined in a sample from the subject, e.g., before administering to the subject an effective amount of a bacterial strain or a composition containing the bacterial strain and/or after administering to the subject an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, detecting the dysbiosis comprises determining that Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased (e.g., reduced) in the sample from subject (e.g., decreased (e.g., reduced) in the gastrointestinal tract of the subject). In some embodiments, detecting the dysbiosis comprises determining that Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased (e.g., reduced) in the sample from subject (e.g., decreased (e.g., reduced) in the gastrointestinal tract of the subject).

In some embodiments, detecting the dysbiosis associated with ulcerative colitis comprises determining that Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased (e.g., enriched) in the sample from subject. In some embodiments, detecting the dysbiosis associated with ulcerative colitis comprises determining that Acinetobacter johnsonii B8, Acinetobacter sp. LMG 995, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased (e.g., enriched) in the sample from subject.

In some embodiments, a method as provided herein can include decreasing a population of an increased (e.g., enriched) bacterial strain in a subject (e.g., a subject with ulcerative colitis). In some embodiments, detecting the decrease in the population of an increased (e.g., enriched) bacterial strain comprises determining the bacterial composition in a sample from the subject (e.g., fecal sample or a biopsy sample such as an intestinal biopsy sample or a colorectal biopsy sample). For example, the bacterial composition can be determined in a sample from the subject before administering to the subject an effective amount of a bacterial strain or a composition containing the bacterial strain and after administering to the subject an effective amount of a bacterial strain or a composition containing the bacterial strain. For example, the population of an increased (e.g., enriched) bacterial strain can be decreased by at least 1%, at least 2%, at least 3%, at least 4%, at least 5%, at least 6%, at least 7%, at least 10%, at least 20%, at least 25%, at least 30%, at least 35%, at least 40%, or at least 50%, e.g., in a sample from the subject after administration of a bacterial strain or a composition containing the bacterial strain to the subject compared to before administration to the subject of an effective amount of a bacterial strain or a composition containing the bacterial strain. Determining the bacterial composition can include, for example, sequencing one or more nucleic acids from the bacteria. In some embodiments, bacteria can be identified by their 16S rRNA gene sequence.

In some embodiments, the increased (e.g., enriched) bacterial strain can be selected from the group consisting of: Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof.

A complete genomic sequence for Acinetobacter johnsonii B8 is available in the GenBank database as, e.g., Accession No. GCF_000368045 and GCF_000949655. In some embodiments, the Acinetobacter johnsonii B8 can have a genomic sequence with at least about 95% sequence identity to the genomic sequence published as one or both of GCF_000368045 and GCF_000949655. For example, Acinetobacter johnsonii B8 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as one or both of GCF_000368045 and GCF_000949655. In some embodiments, Acinetobacter johnsonii B8 can have a 16S RNA gene that is at least 90% identical to one or more (e.g., one, two, three, four, five, six, seven, eight, nine, ten, or all) of SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, and SEQ ID NO:116, and SEQ ID NO:117 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, eight, nine, ten, or all) of SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, and SEQ ID NO:116, and SEQ ID NO:117).

A complete genomic sequence for Acinetobacter sp. LMG 995 is available in the GenBank database as, e.g., Accession No. GCF_000248215 and GCF_000369645. In some embodiments, the Acinetobacter sp. LMG 995 can have a genomic sequence with at least about 95% sequence identity to the genomic sequence published as one of both of GCF_000248215 and GCF_000369645. For example, Acinetobacter sp. LMG 995 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as one or both of GCF_000248215 and GCF_000369645. In some embodiments, Acinetobacter sp. LMG 995 can have a 16S RNA gene that is at least 90% identical to one or more (e.g., one, two, three, four, five, six, seven, or all) of SEQ ID NO:118, SEQ ID NO:119, SEQ ID NO:120, SEQ ID NO:121, SEQ ID NO:122, SEQ ID NO:123, SEQ ID NO:124, and SEQ ID NO:125 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, seven, or all) of SEQ ID NO:118, SEQ ID NO:119, SEQ ID NO:120, SEQ ID NO:121, SEQ ID NO:122, SEQ ID NO:123, SEQ ID NO:124, and SEQ ID NO:125).

A complete genomic sequence for Alicycliphilus denitrificans K601 is available in the GenBank database as, e.g., Accession No. GCF_000204645. In some embodiments, the Alicycliphilus denitrificans K601 can have a genomic sequence with at least about 95% sequence identity to the genomic sequence published as GCF_000204645. For example, Alicycliphilus denitrificans K601 included can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000204645. In some embodiments, Alicycliphilus denitrificans K601 has a 16S RNA gene that is at least 90% identical to one or more (e.g., one, two, three, four, or all) of SEQ ID NO:126, SEQ ID NO:127, SEQ ID NO:128, SEQ ID NO:129, and SEQ ID NO:130. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, or all) of SEQ ID NO:126, SEQ ID NO:127, SEQ ID NO:128, SEQ ID NO:129, and SEQ ID NO:130.

A complete genomic sequence for Aquabacterium parvum B6 is available in the GenBank database as, e.g., Accession No. GCF_001447195. In some embodiments, the Aquabacterium parvum B6 can have a genomic sequence with at least about 95% sequence identity to the genomic sequence published as GCF_001447195. For example, Aquabacterium parvum B6 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_001447195. In some embodiments, Aquabacterium parvum B6 can have a 16S RNA gene that is at least 90% identical to SEQ ID NO:131 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:131).

A complete genomic sequence for Bacteroides plebeius M12 is available in the GenBank database as, e.g., Accession No. GCF_000187895. In some embodiments, the Bacteroides plebeius M12 can have a genomic sequence with at least about 95% sequence identity to the genomic sequence published as GCF_000187895. For example, Bacteroides plebeius M12 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000187895. In some embodiments, Bacteroides plebeius M12 has a 16S RNA gene that is at least 90% identical to one or more (e.g., one, two, or all) of SEQ ID NO:132, SEQ ID NO:133, and SEQ ID NO:134. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, or all) of SEQ ID NO:132, SEQ ID NO:133, and SEQ ID NO:134.

A complete genomic sequence for Bacteroides sp 2_2_4 is available in the GenBank database as, e.g., Accession No. GCF_000157055. In some embodiments, the Bacteroides sp 2_2_4 can have a genomic sequence with at least about 95% sequence identity to the genomic sequence published as GCF_000157055. For example, Bacteroides sp 2_2_4 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000157055. In some embodiments, Bacteroides sp 2_2_4 has a 16S RNA gene that is at least 90% identical to one or both of SEQ ID NO:135 and SEQ ID NO:136. For example, at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or both of SEQ ID NO:135 and SEQ ID NO:136.

In some embodiments, Dechloromonas sp. HZ can have a 16S RNA gene that is at least 90% identical to SEQ ID NO:137 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:137).

A complete genomic sequence for Dialister invisus E7.25 is available in the GenBank database as, e.g., Accession No. GCF_000160055. In some embodiments, the Dialister invisus E7.25 can have a genomic sequence with at least about 95% sequence identity to the genomic sequence published as GCF_000160055. For example, Dialister invisus E7.25 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000160055. In some embodiments, Dialister invisus E7.25 can have a 16S RNA gene that is at least 90% identical to one or more (e.g., one, two, three, four, five, six, or all) of SEQ ID NO:138, SEQ ID NO:139, SEQ ID NO:140, SEQ ID NO:141, SEQ ID NO:142, SEQ ID NO:143, and SEQ ID NO:144 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, six, or all) of SEQ ID NO:138, SEQ ID NO:139, SEQ ID NO:140, SEQ ID NO:141, SEQ ID NO:142, SEQ ID NO:143, and SEQ ID NO:144).

A complete genomic sequence for Micromonas micros ACM 5086 is available in the GenBank database as, e.g., Accession No. GCF_000154405. In some embodiments, the Micromonas micros ACM 5086 can have a genomic sequence with at least about 95% sequence identity to the genomic sequence published as GCF_000154405. For example, Micromonas micros ACM 5086 can have a genomic sequence with at least about 96%, about 97%, about 98%, about 99%, about 99.1%, about 99.2%, about 99.3%, about 99.4%, about 99.5%, about 99.6%, about 99.7%, about 99.8%, or about 99.9% sequence identity to the genomic sequence published as GCF_000154405. In some embodiments, Micromonas micros ACM 5086 can have a 16S RNA gene that is at least 90% identical to one or more (e.g., one, two, three, four, five, or all) of SEQ ID NO:145, SEQ ID NO:146, SEQ ID NO:147, SEQ ID NO:148, SEQ ID NO:149, and SEQ ID NO:150 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to one or more (e.g., one, two, three, four, five, or all) of SEQ ID NO:145, SEQ ID NO:146, SEQ ID NO:147, SEQ ID NO:148, SEQ ID NO:149, and SEQ ID NO:150).

In some embodiments, Sutterella stercoricanis 5BAC4 can have a 16S RNA gene that is at least 90% identical to SEQ ID NO:151 (e.g., at least about 91%, about 91.5% about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.1%, about 99.2%, about 99.3%, about 99.4%, about 99.5%, about 99.6%, about 99.7%, about 99.8%, or about 99.9% identical to SEQ ID NO:151).

In some embodiments, decreasing the population of an increased (e.g., enriched) bacterial strain can include administering a bacteriophage to the subject. See, for example, Sabino et al. Aliment Pharmacol Ther. 51(1):53-63, 2020. In some embodiments, decreasing the population of an increased (e.g., enriched) bacterial strain can include administering to the subject a composition comprising an effective amount of a bacterial strain (e.g., a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof; or a bacterial strain selected from the group consisting of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31).

In some embodiments, methods provided herein can include administering the composition that includes an effective amount of one or more bacterial strains to the subject at least once per day. For example, the composition can be administered two, three, four, or more times per day. In some embodiments, an effective amount of the bacterial strain is administered in one dose, e.g., once per day. In some embodiments, an effective amount of the bacterial strain is administered in more than one dose, e.g., more than once per day. In some embodiments, the method comprises administering the composition to the subject daily, every other day, every three days, or once a week.

In some embodiments, an effective amount of a bacterial strain (e.g., Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof) in a composition described herein can include at least about 1×10³ CFUs of the bacterial strain. For example, an effective amount of a bacterial strain can be at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³to about 1×10¹⁵ CFUs of the bacterial strain (e.g., about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain).

In some embodiments, methods provided herein can include administering a composition comprising a bacterial strain as described herein in combination with one or more other treatments of ulcerative colitis and/or in combination with adjunct therapies such as a therapeutic agent to control pain and/or inflammation. In some embodiments, the treatment of ulcerative colitis can be surgery (e.g., a proctocolectomy). In some embodiments, the treatment of ulcerative colitis and/or adjunct therapy can be a therapeutic agent such as an anti-inflammatory agent, an immunosuppressant, a corticosteroid, or an antibiotic. The composition comprising a bacterial strain and any other treatments and/or adjunct therapies can be administered together (e.g., in the same formulation), or the composition comprising the bacterial strain can be administered concurrently with, prior to, or subsequent to, the one or more other treatments or adjunct therapies.

In some embodiments, a treatment of ulcerative colitis and/or an adjunct therapy administered in combination with a composition comprising a bacterial strain as described herein comprises an anti-inflammatory agent, an immunosuppressant, a corticosteroid, an antibiotic, an anti-diarrheal agent, a pain reliever, an iron supplement, a probiotic, a prebiotic, or a combination thereof. In some embodiments, the anti-inflammatory agent comprises a JAK inhibitor (e.g., tofacitinib, ruxolitinib, and/or baricitinib), sulfasalazine, mesalamine, balsalazide, olsalazine, a corticosteroid, or a combination thereof. In some embodiments, the immunosuppressant comprises azathioprine, mercaptopurine, cyclosporine, infliximab, adalimumab, golimumab, vedolizumab, or a combination thereof. In some embodiments, the corticosteroid comprises prednisone, hydrocortisone, rednisone, budesonide, or a combination thereof. See, for example, Kayal and Shah. J Clin Med. 2019 Dec. 30; 9(1).

In some embodiments, a prebiotic and/or probiotic can be administered in combination with a composition comprising a bacterial strain as described herein. Non-limiting examples of a probiotic include one of more of Bifidobacteria (e.g., B. animalis, B. breve, B. lactis, B. longum, B. longum, or B. infantis), Lactobacillus (e.g., L. acidophilus, L. reuteri, L. bulgaricus, L. lactis, L. casei, L. rhamnosus, L. plantarum, L. paracasei, or L. delbreuckii/bulgaricus), Saccharomyces boulardii, E. coli Nissle 1917, and Streptococcus thermophiles. Non-limiting examples of a prebiotic include a fructooligosaccharide (e.g., oligofructose, inulin, or an inulin-type fructan), a galactooligosaccharide, an amino acid, or an alcohol. See, for example, Ramirez-Farias et al. (2008. Br. J Nutr. 4:1-10) and Pool-Zobel and Sauer (2007. J Nutr. 137:2580-2584).

In some embodiments, an effective amount of the therapeutic agent is administered in combination with a composition comprising a bacterial strain as described herein.

In some embodiments, methods provided herein can include monitoring the subject after treatment with a composition described herein to determine if one or more symptoms have been alleviated, if the severity of one or more symptoms has been decreased (e.g., reduced), or if progression of the disease has been delayed or inhibited in the subject. Non-limiting examples of ulcerative colitis symptoms include: diarrhea, abdominal pain, abdominal cramping, rectal pain, rectal bleeding, urgency to defecate, inability to defecate despite urgency (tenesmus), weight loss, fatigue, fever, and failure to grow (in children).

There are numerous scores and clinical markers that can be utilized to assess the efficacy of administering a composition that includes bacterial strain as described herein in treating an inflammatory disease.

As an example, there are two general approaches to evaluating patients with an inflammatory bowel disease such as ulcerative colitis. The first involves the visual examination of the mucosa and relies on the observation of signs of damage to the mucosa. Any procedure that allows an assessment of the mucosa can be used. Non-limiting examples include a barium enema, an x-ray, and endoscopy. An endoscopy may be of the esophagus, stomach and duodenum (esophagogastroduodenoscopy), small intestine (enteroscopy), or large intestine/colon (colonoscopy, sigmoidoscopy). These techniques can be used to identify an area of inflammation, an ulcer, and/or an abnormal growth such as a polyp. A scoring system based on the visual examination of the GI tract can be used to determine the status and/or severity of IBD. The scoring systems can help ensure the uniform assessment of different subjects, e.g., even if subjects are assessed by different medical professionals. For example, non-limiting examples of evaluations based on visual examination of UC are described and compared in Daperno et al. J Crohns Colitis. 2011 5:484-98. In some embodiments, findings on endoscopy or other examination of the mucosa can be incorporated into a clinical scoring systems. A clinical scoring system can also incorporate data based on one or more symptoms including stool frequency, rectal bleeding, and a physician's global assessment. Some of these scoring systems also take into account a quantitative assessment of the effect on quality of life.

An example of a scoring system for UC is the Mayo scoring system (Schroeder et al., N Eng J Med, 1987, 317:1625-1629). Other non-limiting examples of scoring systems for UC include the Ulcerative Colitis Endoscopic Index of Severity (UCEIS) score (Travis et al, 2012, Gut, 61:535-542), Baron Score (Baron et al., 1964, BMJ, 1:89), Ulcerative Colitis Colonoscopic Index of Severity (UCCIS) (Thia et al., 2011, Inflamm Bowel Dis, 17:1757-1764), Rachmilewitz Endoscopic Index (Rachmilewitz, 1989, BMJ, 298:82-86), Sutherland Index (also known as the UC Disease Activity Index (UCDAI) scoring system; Sutherland et al., 1987, Gastroenterology, 92:1994-1998), Matts Score (Matts, 1961, QJM, 30:393-407), and Blackstone Index (Blackstone, 1984, Inflammatory bowel disease. In: Blackstone MO (ed.) Endoscopic interpretation: normal and pathologic appearances of the gastrointestinal tract, 1984, pp. 464-494). Also see, for example, Paine, 2014, Gastroenterol Rep 2:161-168.

In some embodiments, treatment of ulcerative colitis in a subject can be assessed using one or more indexes or biomarkers selected from the group consisting of: the UC Disease Activity Index, the UC Disease Activity Index, the Mayo Score, Mayo Score, the Harvey-Bradshaw Index, an IBD questionnaire, Manitoba IBD Index, IBD-Control questionnaire, CRP levels, calprotectin levels, fecal lactoferrin levels, an endoscopy, Rachmilewitz Endoscopic Index, Ulcerative Colitis Endoscopi Index of Severity, and CD Digestive Damage Score. For example, an improvement in one or more of the above indexes or biomarkers after administering a bacterial strain, or a composition thereof, as described herein to the subject indicates treatment of the ulcerative colitis. In some embodiments, a reduction in the Mayo Score indicates treatment of UC in a subject. The Mayo Score is a combined endoscopic and clinical scale used to assess the severity of UC and has a scale of 1-12. In some embodiments, treatment comprises a reduction in the Mayo Score by at least 1, 2 or 3 points in at least one of: rectal bleeding, blood streaks seen in the stool, endoscopy subscore, and physician's global assessment.

Diagnosing ulcerative colitis in a subject can include ruling out other possible causes for a subject's symptoms (e.g., an infection caused by a bacteria, virus, or parasite). In some embodiments, diagnosing ulcerative colitis in a subject can include performing one or more tests or procedures such as a blood test (e.g., a test for anemia or a test for an infection using a blood sample form the subject), a stool sample (e.g., a test for white blood cells in stool sample from the subject), a colonoscopy, a biopsy, a sigmoidoscopy, an X-ray, a computerized tomography (CT) scan, a CT enterography, and a magnetic resonance (MR) enterography. Imaging methods such as a colonoscopy, a sigmoidoscopy, an X-ray, a computerized tomography (CT) scan, a CT enterography, and a magnetic resonance (MR) enterography, can be used to determine the level and location of inflammation in the colon of a subject.

In some embodiments, compositions provided herein can include one or more excipients and can be formulated for any of a number of delivery systems suitable for administration to a subject (e.g., probiotic or LBP delivery systems). Non-limiting examples of an excipient include a buffering agent, a diluent, a preservative, a stabilizer, a binding agent, a filler, a lubricant, a dispersion enhancer, a disintegrant, a lubricant, a disintegrant, a wetting agent, a glidant, a flavoring agent, a sweetener, and a coloring agent. For example, in some embodiments, tablets or capsules can be prepared by conventional means with excipients such as binding agents, fillers, lubricants, disintegrants, or wetting agents. Any of the compositions described herein can be administered to a subject to treat ulcerative colitis as described herein.

In some embodiments, a composition as described herein can be formulated for oral delivery. In some embodiments, the composition can be formulated as a tablet, a chewable tablet, a capsule, a stick pack, a powder, effervescent powder, or a liquid. In some embodiments, a composition can include coated beads that contain the bacterial strain. In some embodiments, a powder comprising the bacterial strain can be suspended or dissolved in a drinkable liquid such as water for administration. In some embodiments, the composition is a solid composition.

In some embodiments, a composition described herein can be formulated for various immediate and controlled release profiles of the bacterial strain. For example, a controlled release formulation can include a controlled release coating disposed over the bacterial strain. In some embodiments, the controlled release coating is an enteric coating, a semi-enteric coating, a delayed release coating, or a pulsed release coating. In some embodiments, a coating can be suitable if it provides an appropriate lag in active release (i.e., release of the bacterial strain). For example, in some embodiments, the composition can be formulated as a tablet that includes a coating (e.g., an enteric coating).

In some embodiments, the composition can be formulated for topical delivery. In some embodiments, the composition can be in the form of a paste, gel, cream, spray, suppository, mousse, emollient, ointment, foam, or suspension.

In some embodiments, the bacterial strain in the composition is a culture of a single strain of organism. In some embodiments, the composition comprises a bacterial strain that is isolated. In some embodiments, the bacterial strain is isolated and cultured in vitro to increase the number or concentration of the bacterial strain. Increasing the number or concentration of the bacterial strain can be useful, for example, to enhance the efficacy of a composition comprising the bacterial strain.

In some embodiments, an effective amount of the bacterial strain in a composition described herein comprises at least about 1×10³ CFU of the bacterial strain. For example, at least about 1×10³, about 1×10⁴, about 1×10⁵, about 1×10⁶, about 1×10⁷, about 1×10⁸, about 1×10⁹, about 1×10¹⁰, about 1×10¹¹, about 1×10¹², about 1×10¹³, or about 1×10¹⁴ CFUs of the bacterial strain. In some embodiments, the effective amount of a bacterial strain in a composition described herein comprises about 1×10³ to about 1×10¹⁵ CFUs of the bacterial strain. For example, about 1×10³ to about 1×10⁶, about 1×10³ to about 1×10⁸, about 1×10³ to about 1×10¹⁰, about 1×10³ to about 1×10¹², about 1×10³ to about 1×10¹⁴, about 1×10⁷ to about 1×10¹², about 1×10¹³ to about 1×10¹⁵, about 1×10¹¹ to about 1×10¹⁵, about 1×10⁹ to about 1×10¹⁵, about 1×10⁷ to about 1×10¹⁵, or about 1×10⁵ to about 1×10¹⁵ CFUs of the bacterial strain.

In some embodiments, the composition can include one or more biologically pure strains (e.g., two or more, three or more, four or more, five or more, six or more, seven or more, eight or more, nine or more, ten or more, eleven or more, twelve or more, thirteen or more, fourteen or more, or fifteen or more bacterial strains). For example, the composition can include biologically pure Bacillus sp. BT1B_CT2, biologically pure Bacteroides coprocola M16, biologically pure Bacteroides eggerthii CCUG 9559, biologically pure Bacteroides vulgatus 8482, biologically pure Butyricimonas virosa MT12, biologically pure Coprococcus eutactus ATCC 27759, biologically pure Desulfovibrio piger DSM 749, biologically pure Dolosigranulum pigrum IFO 15550, biologically pure Eubacterium biforme DSM 3989, biologically pure Eubacterium eligens DSM 3376, biologically pure Fusobacterium.prausnitzii ATCC 27768, biologically pure Howardella ureilytica GPC 589, biologically pure Megasphaera elsdenii LC1, biologically pure Parabacteroides johnsonii M-165, biologically pure Phascolarctobacterium sp. YIT 12067, biologically pure Roseburia hominus A2-183, biologically pure Ruminococcus callidus VPI 57-31, or any combination thereof.

In some embodiments, the composition is a solid composition that includes at least 1×10³ CFUs of a bacterial strain (e.g., a biologically pure strain) and one or more excipients. Identifying characteristics of suitable strains, including homology to 16S rRNA sequences are described above.

In some embodiments, each member of the same bacterial strain has a 16S rRNA gene sequence with at least about 90%, about 91%, about 92%, about 93%, about 94%, about 95%, about 96%, about 97%, about 98%, about 99%, about 99.5%, about 99.6%, about 99.7%, about 99.8%, or about 99.9% sequence identity to the 16S rRNA gene sequence of each other member of the same bacterial strain.

In some embodiments, a bacterial strain in a composition described herein is preserved. Methods for preserving bacterial strains can include lyophilization and cryopreservation, optionally in the presence of a protectant. Non-limiting examples of protectants include sucrose, inulin, and glycerol. In some embodiments, a composition can include a lyophilized or cryopreserved bacterial strain such as Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, and an optional protectant. In some embodiments, a composition can include a lyophilized or cryopreserved bacterial strain such as Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, and an optional protectant.

In some embodiments, wherein the bacterial strain is a combination of two or more of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are lyophilized or cryopreserved.

In some embodiments, the composition is a live bacterial product (LBP). In some embodiments, the bacterial strain in the composition is viable. The viable bacterial strain can be, for example, cryopreserved and/or lyophilized. In some embodiments, a composition for delivery of live bacterial strains (e.g., Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof), can be formulated to maintain viability of the bacterial strain.

In some embodiments, wherein the bacterial strain is a combination of two or more of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are viable.

In some embodiments, the composition comprises elements that protect the bacterial strain from the acidic environment of the stomach (e.g., an enteric coating).

In some embodiments, the bacterial strain in the composition can be non-viable. In some embodiments, the non-viable bacterial strain is heat-killed, irradiated, or lysed.

In some embodiments, wherein the bacterial strain is a combination of two or more of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 one or more of Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, and Ruminococcus callidus VPI 57-31 are non-viable (e.g., heat-killed, irradiated, or lysed).

In some embodiments, the bacterial strain as described herein may be used in prophylactic applications. For example, in a prophylactic application, a bacterial strain or a composition described herein can be administered to a subject susceptible to, or otherwise at risk of, a particular disease in an amount that is sufficient to at least partially reduce the risk of developing a disease. One of ordinary skill in the art will appreciate that the precise amounts of the bacterial strain administered may depend on a number of subject specific factors such as the subject's state of health and/or weight.

Also provided herein are methods of identifying a subject as having or having an increased likelihood of developing ulcerative colitis that include (a) identifying a subject having a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or (ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as having or having an increased likelihood of developing ulcerative colitis; or (b) identifying a subject having a sample that does not have (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or (ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as not having or not having an increased likelihood of developing ulcerative colitis.

Also provided herein are methods of diagnosing a subject as having ulcerative colitis that include (a) identifying a subject having a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or (ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as having ulcerative colitis; or b) identifying a subject having a sample that does not have (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as not having ulcerative colitis.

Also provided herein are methods of treating ulcerative colitis in a subject that include a) administering a ulcerative colitis therapy to a subject determined to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus; or b) not administering a ulcerative colitis therapy to a subject determined not to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

Also provided herein are methods for treating a subject in need thereof that include a) administering a composition comprising an effective amount of a bacterial species selected from the group consisting of Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus; or b) not administering a composition comprising an effective amount of a bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined not to have a sample that has (i) an increased level of one or more (e.g., two or more, three or more, four or more, five or more, six or more, seven or more, eight or more, nine or more, or ten) bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of one or more (e.g., two or more, three or more, four or more, five or more, six or more, seven or more, eight or more, nine or more, ten or more, eleven or more, twelve or more, thirteen or more, fourteen or more, fifteen or more, sixteen or more, or seventeen) bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus. In some embodiments, the subject has ulcerative colitis.

Some embodiments of any of the methods described herein include detecting the level of one or more bacterial species in the sample from the subject. In some embodiments of any of the methods described herein, the level of Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, or Sutterella stercoricanis is increased in comparison to the same bacterial species in a reference sample.

Some embodiments of any of the methods described herein include determining that the sample has (i) an increased level of two or more (e.g., three or more, four or more, five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of two or more (e.g., three or more, four or more, five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

Some embodiments of any of the methods described herein include determining that the sample has (i) an increased level of three or more (e.g., four or more, five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of three or more (e.g., four or more, five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

Some embodiments of any of the methods described herein include determining that the sample has (i) an increased level of four or more (e.g., five or more, six or more, seven or more, eight or more, nine or more, or ten) of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or ii) a decreased level of four or more (e.g., five or more, six or more, seven or more, eight or more, nine or more, ten or more, eleven or more, twelve or more, thirteen or more, fourteen or more, fifteen or more, sixteen or more, or seventeen) of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium prausnitzii, Howardella ureilytica, Megasphaera elsdenii, Parabacteroides johnsonii, Phascolarctobacterium sp., Roseburia hominus, and Ruminococcus callidus.

The invention will be further described in the following examples, which do not limit the scope of the invention described in the claims.

### EXAMPLES

### Example 1. Identification of Bacterial Strains Associated with Ulcerative Colitis

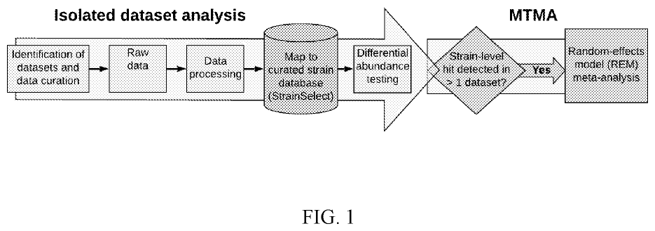

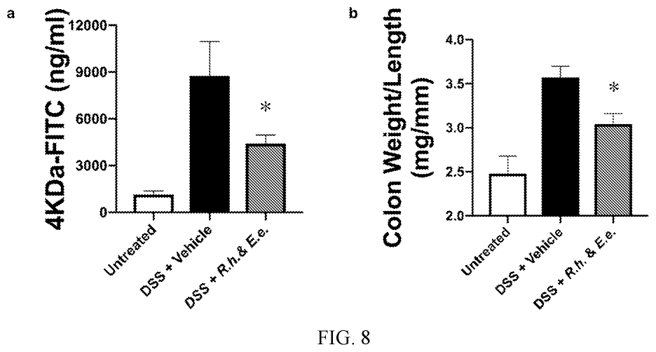

A multiple-technology meta-analysis (MTMA) approach was applied to datasets, which led to the identification of strains unique to colorectal cancer. To overcome challenges of comparing clinical variables across datasets from multiple institutes, a controlled vocabulary of hierarchically organized terms and manually re-annotated metadata from public datasets using this vocabulary was created. FIG. 8 provides the details regarding the cohorts, datasets, and contrasts analyzed to identify differentially abundant (DA) taxa correlating with disease progression. Statistical analysis of each dataset was performed using the workflow described in FIG. 1. Strain-level annotation was achieved using StrainSelect, a database containing sequence information of bacterial and archaeal strains connected to genome identifiers, which facilitated comparative analysis of taxa abundances at a strain-level across datasets.

Methods

Patient Populations

Mucosal biopsies were collected at the University of Calgary during initial diagnosis or follow up endoscopy from September 2007 and April 2013 (BioProject accession number PRJNA527097). A diagnosis of ulcerative colitis was confirmed by a qualified gastroenterologist at the Foothills Medical Center, University of Calgary. Informed consent for use of biopsy samples was obtained by the University of Calgary Intestinal Inflammation Tissue Bank and the study was approved by the University of Calgary, Conjoint Health Research Ethics Board (ID:18142 and 14-2429).

Procurement of Raw Data and Metadata Curation

DNA was isolated from samples using the MoBio Ultraclean Tissue and Cells DNA isolation kit (MoBio Laboratories, Carlsbad, Calif.). For 16S-NGS, the V4 region of the 16S rRNA gene was PCR-amplified using fusion primers designed against surrounding conserved regions and tailed to incorporate Illumina adaptors and indexing barcodes as described previously (Caporaso, J. G. et al. ISME J. 6, 1621-1624 (2012)). Amplicons were sequenced on the Illumina MiSeq (Illumina, San Diego, Calif.). For 16S-Hyb, V1 though V9 16S rRNA gene analysis was performed on the G3 PhyloChip using lab protocols and image-scoring procedures previously described (Sarhan, M. S. et al. 33, 317-325 (2018)). The G3 PhyloChip design was described in Hazen et al. and Mendes et al. (Hazen, T. C. et al. Science 330, 204-8 (2010) and Mendes, R. et al. Science. 332, 1097-1100 (2011)).

Fastq/Fasta files and metadata were procured from public repositories. Metadata stored with raw data, such as NCBI's RunInfo table associated with the SRA Run Selector, and/or metadata published in tables in the primary text or supplementary files of the publication, were retrieved and manually re-annotated using a controlled vocabulary of hierarchically organized terms. An in-house database was created to store all study-related data and facilitate appropriate metadata annotation of all datasets via manual curation. Clinical metadata was stored in this database as a series of label:value pairs attached to the biospecimen from which the data files were generated.

Processing, Strain Annotation and Statistical Analyses of Raw Data

16S rRNA Sequencing Datasets (16S-NGS and 16S-Sanger)

Paired-end reads were merged using USEARCH and resulting sequences were compared to the StrainSelect database version 2014 (SS14) using USEARCH (usearch_global) (Edgar, R. C. Bioinformatics 26, 2460-2461 (2010) to generate strain-level abundance information. StrainSelect is a repository of strain identifiers obtained from gene sequencing, genome sequencing, draft genomes, and metagenomic assemblies of known prokaryotic strains (StrainSelect, www.secondgenome.com/platform/data-analysis-tools/strainselect). All sequences matching a unique strain at an identity ≥99% was assigned a strain-level annotation. To ensure specificity of these strain matches, a difference of ≥0.25% between the identity of the best match and the second best match was required (e.g., 99.75 vs. 99.5).

For each strain OTU, a single read was selected to represent all sequences belonging to that OTU and it was mapped by USEARCH (usearch_global) against all other strain OTU representatives to infer strain abundance(s). Remaining non-strain sequences were quality filtered and dereplicated with USEARCH, and clustered at ≥97% similarity via UPARSE (Allali, I. et al. doi:10.1186/s12866-017-1101-8) (de novo OTU clustering). A representative consensus sequence was then assigned to each de novo OTU. As the UPARSE clustering algorithm performs chimera filtering, all OTUs likely to be chimeric in nature were discarded and omitted from consideration. All non-strain sequences that passed quality filtering were mapped to their corresponding representative consensus sequences, enabling the generation of abundance tables for de novo OTUs alongside strain abundances.

Any sample yielding fewer than 15 sequence reads was removed from consideration prior to statistical analysis. For all datasets, downstream of a 5% prevalence filter, DESeq2⁴⁴ was used to calculate significant differences across all bins (OTUs, and strains) and adjusted p-values were determined with the Benjamini-Hochberg correction. Significant results were determined as adjusted p-values <0.05. Log 2 fold change and standard error were calculated via DESeq2 and applied to subsequent analyses.

PhylaChip Datasets (16S-Hyb)

Emperical OTUs (eOTUs) generated using image-scoring procedures were annotated against SS14 to obtain strain-level annotations as described previously (Sarhan, et al. Microbes Environ. 33, 317-325 (2018)).

Downstream of a 5% prevalence filter, significant differences for all eOTUs were calculated via Welch's t-tests, and adjusted p-values were determined with the Benjamini-Hochberg correction. Significant results were determined as adjusted p-values <0.05. Fold change and variance were calculated using the metafor package in R⁴⁵. Standard error was calculated as the square root of the variance and both fold change and standard error were extrapolated to a log 2 scale.

Random-Effects Model

Log 2 fold change, p-values and standard errors pertaining to per-dataset statistical results in each disease area were integrated in MTMA using a Random effects model, generated using the metafor R package. Only bins with strain-level annotations in each dataset, and only those strains observed in at least two datasets, post prevalence filtering, were retained for REM analysis. False discovery correction for REM generated p-values was achieved using the Benjamini-Hochberg method. Throughout this paper, differences are deemed to be statistically significant at adjusted (Benjamini-Hochberg corrected) p-values <0.05 in both isolated dataset analysis and MTMA.

### Results

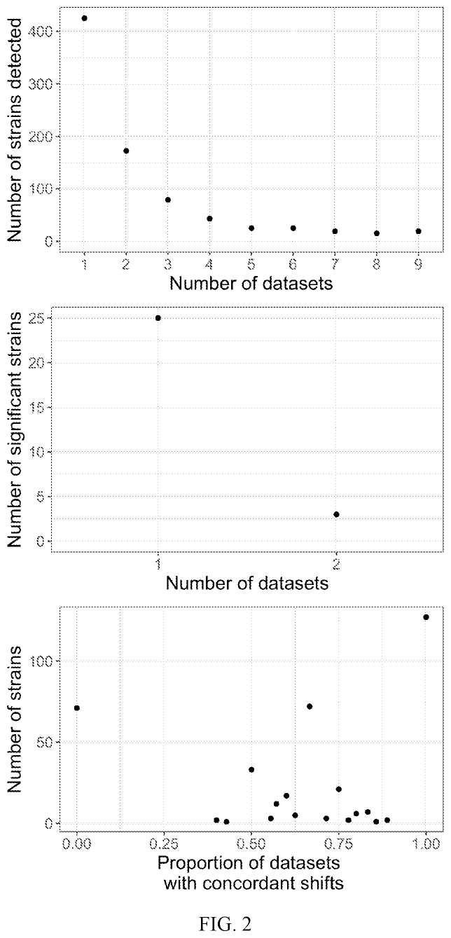

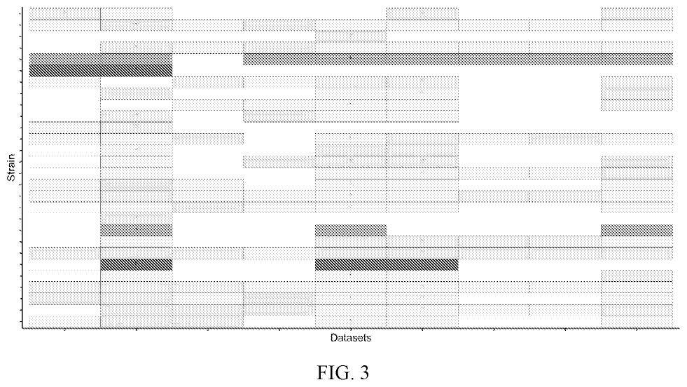

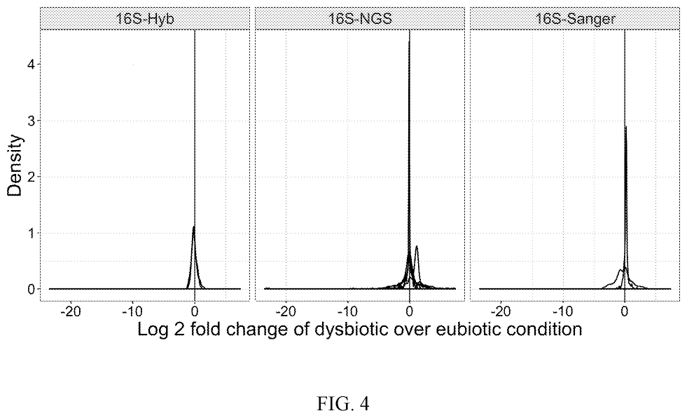

Identification of differentially abundant strains across cohorts from a simple comparison of isolated datasets was limited as less than 3% of the strains were detected across all datasets (FIG. 2, top panel). Further, while isolated analysis identified between 28 strains as significantly differentially abundant within a disease, these strain-disease associations were mostly cohort-specific (FIG. 2, middle panel) and in many cases not supported in trend, i.e a strain being consistently associated with either homeostasis or dysbiosis, across other datasets (FIG. 3; light-shaded rows). Variation was also observed in the magnitude of differential abundance derived from the cohorts and especially the profiling technologies (log 2 fold change; FIG. 4). As independent analyses of individual microbiome datasets detect signals specific to a particular dataset, they are intrinsically biased by differences in cohort or experimental design. This, coupled with sparsity in overlap of significantly-DA strains identified across datasets and variation in effect sizes across DNA-profiling technologies, underscores the need for more robust means of discerning concordant signatures across datasets.

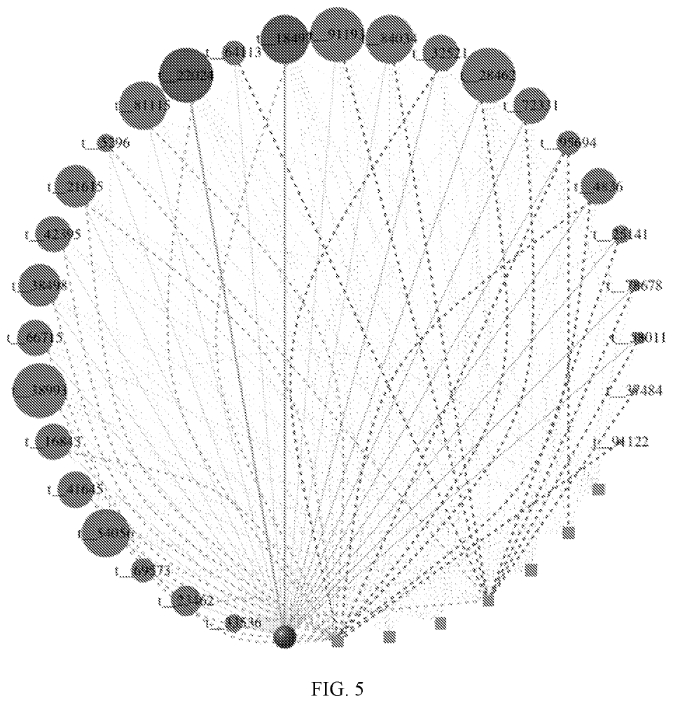

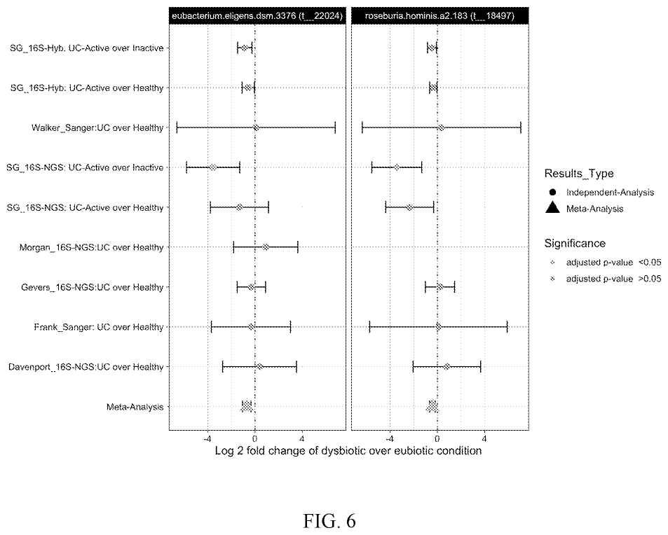

Strain-level results were integrated from isolated analyses in each disease via MTMA as described in FIG. 1. Significant strain-disease associations were identified only when the direction of differential abundance of the strain was supported by multiple datasets (FIG. 5; blue strains connected to MTMA nodes via thick-solid lines). In most cases, significantly-differentially abundant strains identified by MTMA demonstrated concordance in the direction of their shifts in respective isolated analyses (FIG. 6). In diseases with highly heterogeneous populations such as ulcerative colitis, MTMA identified significant disease associations with less concordance in direction of differential abundance across cohorts. By accounting for variations in measurements (large confidence intervals) across datasets, MTMA identified two strains significantly decreased (e.g., reduced) in ulcerative colitis even where most isolated analyses failed to significantly identify the associations (FIG. 6).

The associations in isolated analyses that were not supported in trend by other datasets were not significant by MTMA (FIG. 5; dark-green circles). Several strain-disease associations identified in MTMA were not identified in isolated analyses of the datasets (FIG. 5; purple dots). Thus, MTMA corroborates findings from isolated analysis if supported across datasets but eliminates if discordant, and MTMA identifies novel disease-strain associations that isolated analyses failed to detect.

While previous meta-analyses have correlated higher-order taxa with disease, pin-pointing the biological relevance of these findings can be challenging as these taxa encompass organisms with varied biological functions. For example while isolated and meta-analysis have associated genera within Ruminococcacaea and Lachnospiracaea with inflammatory bowel disease, translating this association to worsened colonic health from a reduction of butyrate producing bacteria is challenging as these genera encompass species not all of which are butyrate producers (Duvallet et al. Nat. Commun. 8, 1784 (2017)). However, the strain-level bacterial signatures of disease identified herein offers the resolution required for deciphering the biological function of bacteria in modulating disease.

MTMA can enable synthesis of existing knowledge of the microbiome, and the approach as shown in FIG. 1 can facilitate comparative analysis of taxa abundances at a strain-level across datasets generated with different DNA-profiling technologies. Harnessing the MTMA framework, with its ability to integrate datasets across DNA-profiling technologies and pinpoint specific strains, can allow for identification of robust microbiome modulators of disease by integrating the growing body of evidence on the role played by microbiome in disease.

### Other Embodiments

It is to be understood that while the invention has been described in conjunction with the detailed description thereof, the foregoing description is intended to illustrate and not limit the scope of the invention which is defined by the scope of the appended claims. Other aspects, advantages, and modification are within the scope of the following claims.

What is claimed is:

1. A method of identifying a subject as having or having an increased likelihood of developing ulcerative colitis, the method comprising:
(a) identifying a subject having a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as having or having an increased likelihood of developing ulcerative colitis; or
(b) identifying a subject having a sample that does not have:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as not having or not having an increased likelihood of developing ulcerative colitis.

2. A method of diagnosing a subject as having ulcerative colitis, the method comprising:
(a) identifying a subject having a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as having ulcerative colitis; or
(b) identifying a subject having a sample that does not have:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as not having ulcerative colitis.

3. A method of treating ulcerative colitis in a subject, the method comprising:
(a) administering a ulcerative colitis therapy to a subject determined to have a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus; or
(b) not administering a ulcerative colitis therapy to a subject determined not to have a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus.

4. A method for treating a subject in need thereof, the method comprising:
(a) administering a composition comprising an effective amount of a bacterial species selected from the group consisting of Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined to have a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus; or
(b) not administering a composition comprising an effective amount of a bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus, as a monotherapy, or in conjunction with another ulcerative colitis therapy, to a subject determined not to have a sample that has:
(i) an increased level of one or more bacterial species selected from the group consisting of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of one or more bacterial species selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus.

5. The method of claim 4, wherein the subject has ulcerative colitis.

6. The method of any one of claims 1-5, wherein the method comprises detecting the level of one or more bacterial species in the sample from the subject.

7. The method of any one of claims 1-6, wherein the level of Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, or Sutterella stercoricanis is increased in comparison to the same bacterial species in a reference sample.

8. The method of any one of claims 1-7, wherein the method comprises determining that the sample has:
(i) an increased level of two or more of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of two or more of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus.

9. The method of any one of claims 1-8, wherein the method comprises determining that the sample has:
(i) an increased level of three or more of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of three or more of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus.

10. The method of any one of claims 1-9, wherein the method comprises determining that the sample has:
(i) an increased level of four or more of: Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, and Sutterella stercoricanis; and/or
(ii) a decreased level of four or more of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, and Ruminococcus callidus.

11. A method for treating ulcerative colitis in a subject, the method comprising administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, Ruminococcus callidus, and a combination thereof.

12. A method for treating ulcerative colitis in a subject, the method comprising:
(a) detecting a dysbiosis associated with ulcerative colitis in a sample from the subject; and
(b) administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, Ruminococcus callidus, and a combination thereof.

13. The method of claims 1-10 and 12, wherein the sample is a biopsy sample.

14. The method of claim 13, wherein the sample is a colorectal biopsy sample.

15. The method of claim 13, wherein the sample is a fecal sample.

16. The method of any one of claims 12-15, wherein detecting the dysbiosis associated with ulcerative colitis comprises determining bacterial gene expression in the sample from the subject.

17. The method of any one of claims 12-16, wherein detecting the dysbiosis associated with ulcerative colitis comprises determining bacterial composition in the sample from the subject.

18. The method of claim any one of claims 12-17, wherein detecting the dysbiosis associated with ulcerative colitis comprises determining that Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, Sutterella stercoricanis, or a combination thereof, is increased in the sample from subject.

19. The method of claim any one of claims 12-18, wherein detecting the dysbiosis associated with ulcerative colitis comprises determining that Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, Ruminococcus callidus, or a combination thereof, is decreased in the sample from subject.

20. The method of claim 19, wherein Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, Ruminococcus callidus, or a combination thereof, is decreased in the gastrointestinal tract of the subject.

21. A method for treating a subject in need thereof, the method comprising decreasing a population of an increased bacterial strain in the subject, wherein the increased bacterial strain is selected from the group consisting of Acinetobacter johnsonii, Acinetobacter sp., Alicycliphilus denitrificans, Aquabacterium parvum, Bacteroides plebeius, Bacteroides sp, Dechloromonas sp., Dialister invisus, Micromonas micros, Sutterella stercoricanis, and a combination thereof.

22. The method of claim 21, wherein the subject has ulcerative colitis.

23. The method of claim 21 or claim 22, wherein decreasing the population of an increased bacterial strain comprises administering to the subject a bacteriophage.

24. The method of any one of claims 21-23, wherein decreasing the population of an increased bacterial strain comprises administering to the subject a composition comprising an effective amount of a bacterial strain selected from the group consisting of: Bacillus sp., Bacteroides coprocola, Bacteroides eggerthii, Bacteroides vulgatus, Butyricimonas virosa, Coprococcus eutactus, Desulfovibrio piger, Dolosigranulum pigrum, Eubacterium biforme, Eubacterium eligens, Fusobacterium.prausnitzii, Howardella ureilytica, Megasphaera elsdenii, Parabacteroides johnsonii, Phascolarctobacterium sp., Roseburia hominus, Ruminococcus callidus, and a combination thereof.

25. The method of any one of claims 1-20 and 22-24, wherein the ulcerative colitis is distal colitis.

26. The method of any one of claims 1-20 and 22-24, wherein the ulcerative colitis is extensive colitis.

27. The method of any one of claims 1-20 and 22-24, wherein the ulcerative colitis is selected from the group consisting of: ulcerative proctitis, left-sided colitis, proctosigmoiditis, and pancolitis.

28. The method of any one of claims 1-20 and 24-27, wherein the bacterial species Bacillus sp. comprises the bacterial strain Bacillus sp. BT1B_CT2.

29. The method of any one of claims 1-20 and 24-28, wherein the bacterial species Bacteroides coprocola comprises the bacterial strain Bacteroides coprocola M16.

30. The method of any one of claims 1-20 and 24-29, wherein the bacterial species Bacteroides coprocola comprises the bacterial strain c CCUG 9559.

31. The method of any one of claims 1-20 and 24-30, wherein the bacterial species Bacteroides vulgatus comprises the bacterial strain Bacteroides vulgatus 8482.

32. The method of any one of claims 1-20 and 24-31, wherein the bacterial species Butyricimonas virosa comprises the bacterial strain Butyricimonas virosa MT12.

33. The method of any one of claims 1-20 and 24-32, wherein the bacterial species Coprococcus eutactus comprises the bacterial strain Coprococcus eutactus ATCC 27759.

34. The method of any one of claims 1-20 and 24-33, wherein the bacterial species Desulfovibrio piger comprises the bacterial strain Desulfovibrio piger DSM 749.

35. The method of any one of claims 1-20 and 24-34, wherein the bacterial species Dolosigranulum pigrum comprises the bacterial strain Dolosigranulum pigrum IFO 15550.

36. The method of any one of claims 1-20 and 24-35, wherein the bacterial species Eubacterium biforme comprises the bacterial strain Eubacterium biforme DSM 3989.

37. The method of any one of claims 1-20 and 24-36, wherein the bacterial species Eubacterium eligens comprises the bacterial strain Eubacterium eligens DSM 3376.

38. The method of any one of claims 1-20 and 24-37, wherein the bacterial species Fusobacterium prausnitzii comprises the bacterial strain Fusobacterium.prausnitzii ATCC 27768.

39. The method of any one of claims 1-20 and 24-38, wherein the bacterial species Howardella ureilytica comprises the bacterial strain Howardella ureilytica GPC 589.

40. The method of any one of claims 1-20 and 24-39, wherein the bacterial species Megasphaera elsdenii comprises the bacterial strain Megasphaera elsdenii LC1.

41. The method of any one of claims 1-20 and 24-40, wherein the bacterial species Parabacteroides johnsonii comprises the bacterial strain Parabacteroides johnsonii M-165.

42. The method of any one of claims 1-20 and 24-41, wherein the bacterial species Phascolarctobacterium sp. comprises the bacterial strain Phascolarctobacterium sp. YIT 12067.

43. The method of any one of claims 1-20 and 24-42, wherein the bacterial species Roseburia hominus comprises the bacterial strain Roseburia hominus A2-183.

44. The method of any one of claims 1-20 and 24-43, wherein the bacterial species Ruminococcus callidus comprises the bacterial strain Ruminococcus callidus VPI 57-31.

45. The method of any one of claims 1-20 and 24-44, wherein the bacterial strain is selected from the group consisting of: Roseburia hominus A2-183, Eubacterium eligens DSM 3376, and a combination thereof.

46. The method of any one of claims 27-45, wherein the bacterial strain improves intestinal barrier function of the subject.

47. The method of any one of claims 43-46, wherein strain Roseburia hominus A2-183 has a 16S RNA gene that is at least 95% identical to SEQ ID NO:99.

48. The method of any one of claims 43-46, wherein strain Roseburia hominus A2-183 has a 16S RNA gene that is at least 95% identical to SEQ ID NO:100.

49. The method of any one of claims 43-46, wherein strain Roseburia hominus A2-183 has a 16S RNA gene that is at least 95% identical to SEQ ID NO:101.

50. The method of any one of claims 43-46, wherein strain Roseburia hominus A2-183 has a 16S RNA gene that is at least 95% identical to SEQ ID NO:102.

51. The method of any one of claims 43-46, wherein strain Roseburia hominus A2-183 has a 16S RNA gene that is at least 95% identical to SEQ ID NO:103.

52. The method of any one of claims 43-46, wherein strain Roseburia hominus A2-183 has a 16S RNA gene that is at least 95% identical to SEQ ID NO:104.

53. The method of any one of claims 37-46, wherein strain Eubacterium eligens DSM 3376 has a 16S RNA gene that is at least 90% identical to SEQ ID NO:73.

54. The method of any one of claims 37-53, wherein strain Eubacterium eligens DSM 3376 has a 16S RNA gene that is at least 95% identical to SEQ ID NO:74.

55. The method of any one of claims 37-53, wherein strain Eubacterium eligens DSM 3376 has a 16S RNA gene that is at least 95% identical to SEQ ID NO:75.

56. The method of any one of claims 37-53, wherein strain Eubacterium eligens DSM 3376 has a 16S RNA gene that is at least 90% identical to SEQ ID NO:76.

57. The method of any one of claims 37-53, wherein strain Eubacterium eligens DSM 3376 has a 16S RNA gene that is at least 95% identical to SEQ ID NO:77.

58. The method of any one of claims 37-53, wherein strain Eubacterium eligens DSM 3376 has a 16S RNA gene that is at least 95% identical to SEQ ID NO:78.

59. The method of any one of claims 37-53, wherein strain Eubacterium eligens DSM 3376 has a 16S RNA gene that is at least 95% identical to SEQ ID NO:79.

60. The method of any one of claims 27-59, wherein the bacterial strain in the composition is viable.

61. The method of any one of claims 27-60, wherein the bacterial strain is lyophilized.

62. The method of any one of claims 11-20 and 24-61, wherein the composition further comprises one or more cryopreservants.

63. The method of any one of claims 27-62, wherein the effective amount of the bacterial strain comprises at least about 1×10³ colony forming units (CFU) of the bacterial strain.

64. The method of any one of claims 24-63, wherein the effective amount of the bacterial strain comprises about 1×10⁴ to about 1×10¹⁵ CFU of the bacterial strain.

65. The method of any one of claims 27-62, wherein the effective amount of the bacterial strain comprises about 1×10⁶ to about 1×10¹⁰ CFU of the bacterial strain.

66. The method of any one of claims 27-66, wherein the bacterial strain in the composition is non-viable.

67. The method of claim 66, wherein the non-viable bacterial strain is heat-killed, irradiated, or lysed.

68. The method of any one of claims 27-67, wherein the method comprises administering the composition to the subject once, twice, or three times per day.

69. The method of any one of claims 27-68, wherein the composition is formulated for oral administration.

70. The method of any one of claims 27-69, wherein the composition is formulated for rectal administration.

71. The method of any one of claims 27-70, wherein the composition is formulated as a tablet, a capsule, a powder, or a liquid.

72. The method of any one of claims 27-71, wherein the composition is formulated as a tablet.

73. The method of claim 72, wherein the tablet is coated.

74. The method of claim 73, wherein the coating comprises an enteric coating.

75. The method of any one of claims 11-74, wherein the method further comprises administering another treatment for ulcerative colitis and/or adjunct therapy to the subject.

76. The method of claim 75, wherein the composition comprising the bacterial strain and the treatment for ulcerative colitis and/or adjunct therapy are administered simultaneously.

77. The method of claim 75, wherein the composition comprising the bacterial strain and the treatment for ulcerative colitis and/or adjunct therapy are administered sequentially.

78. The method of any one of claims 75-77, wherein the treatment for ulcerative colitis and/or adjunct therapy comprises a probiotic.

79. The method of any one of claims 75-78, wherein the treatment for ulcerative colitis and/or adjunct therapy comprises a therapeutic agent.

80. The method of claim 79, wherein the therapeutic agent comprises an anti-inflammatory agent, an immunosuppressant, an antibiotic, an anti-diarrheal agent, a pain reliever, an iron supplement, or a combination thereof.

81. The method of claim 80, wherein the anti-inflammatory agent comprises sulfasalazine, mesalamine, balsalazide, olsalazine, prednisone, hydrocortisone, rednisone, budesonide or a combination thereof.

82. The method of claim 79 or 80, wherein the immunosuppressant comprises azathioprine, mercaptopurine, cyclosporine, infliximab, adalimumab, golimumab, vedolizumab, or a combination thereof.

83. The method of any one of claims 79-82, wherein the composition comprising the bacterial strain further comprises the therapeutic agent.

84. The method of any one of claims 1-83, wherein the subject is a human.

85. A method for treating a subject in need thereof, the method comprising administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

86. The method of claim 85, wherein the subject has ulcerative colitis.

87. A method for treating ulcerative colitis in a subject, the method comprising administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

88. A method for treating ulcerative colitis in a subject, the method comprising:
(a) detecting a dysbiosis associated with ulcerative colitis in a sample from the subject; and
(b) administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

89. The method of claim 88, wherein the sample is a biopsy sample.

90. The method of claim 89, wherein the sample is a colorectal biopsy sample.

91. The method of claim 88, wherein the sample is a fecal sample.

92. The method of any one of claims 88-91, wherein detecting the dysbiosis associated with ulcerative colitis comprises determining bacterial gene expression in the sample from the subject.

93. The method of any one of claims 88-92, wherein detecting the dysbiosis associated with ulcerative colitis comprises determining bacterial composition in the sample from the subject.

94. The method of claim any one of claims 88-93, wherein detecting the dysbiosis associated with ulcerative colitis comprises determining that Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC4, or a combination thereof, is increased in the sample from subject.

95. The method of claim any one of claims 88-94, wherein detecting the dysbiosis associated with ulcerative colitis comprises determining that Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased in the sample from subject.

96. The method of claim 95, wherein Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, or a combination thereof, is decreased in the gastrointestinal tract of the subject.

97. A method for treating a subject in need thereof, the method comprising decreasing a population of an increased bacterial strain in the subject, wherein the increased bacterial strain is selected from the group consisting of Acinetobacter johnsonii B8, Acinetobacter sp. LMG 995, Alicycliphilus denitrificans K601, Aquabacterium parvum B6, Bacteroides plebeius M12, Bacteroides sp 2_2_4, Dechloromonas sp. HZ, Dialister invisus E7.25, Micromonas micros ACM 5086, Sutterella stercoricanis 5BAC45, and a combination thereof.

98. The method of claim 97, wherein the subject has ulcerative colitis.

99. The method of claim 97 or claim 98, wherein decreasing the population of an increased bacterial strain comprises administering to the subject a bacteriophage.

100. The method of any one of claims 97-99, wherein decreasing the population of an increased bacterial strain comprises administering to the subject a composition comprising an effective amount of a bacterial strain selected from the group consisting of: Bacillus sp. BT1B_CT2, Bacteroides coprocola M16, Bacteroides eggerthii CCUG 9559, Bacteroides vulgatus 8482, Butyricimonas virosa MT12, Coprococcus eutactus ATCC 27759, Desulfovibrio piger DSM 749, Dolosigranulum pigrum IFO 15550, Eubacterium biforme DSM 3989, Eubacterium eligens DSM 3376, Fusobacterium.prausnitzii ATCC 27768, Howardella ureilytica GPC 589, Megasphaera elsdenii LC1, Parabacteroides johnsonii M-165, Phascolarctobacterium sp. YIT 12067, Roseburia hominus A2-183, Ruminococcus callidus VPI 57-31, and a combination thereof.

101. The method of any one of claims 86-96 and 98-100, wherein the ulcerative colitis is distal colitis.

102. The method of any one of claims 86-96 and 98-100, wherein the ulcerative colitis is extensive colitis.

103. The method of any one of claims 86-96 and 98-100, wherein the ulcerative colitis is selected from the group consisting of: ulcerative proctitis, left-sided colitis, proctosigmoiditis, and pancolitis.

104. The method of any one of claims 85-96 and 100-103, wherein the bacterial strain comprises Bacillus sp. BT1B_CT2.

105. The method of any one of claims 85-96 and 100-104, wherein the bacterial strain comprises Bacteroides coprocola M16.

106. The method of any one of claims 85-96 and 100-105, wherein the bacterial strain comprises Bacteroides eggerthii CCUG 9559.

107. The method of any one of claims 85-96 and 100-106, wherein the bacterial strain comprises Bacteroides vulgatus 8482.

108. The method of any one of claims 85-96 and 100-107, wherein the bacterial strain comprises Butyricimonas virosa MT12.

109. The method of any one of claims 85-96 and 100-108, wherein the bacterial strain comprises Coprococcus eutactus ATCC 27759.

110. The method of any one of claims 85-96 and 100-109, wherein the bacterial strain comprises Desulfovibrio piger DSM 749.

111. The method of any one of claims 85-96 and 100-110, wherein the bacterial strain comprises Dolosigranulum pigrum IFO 15550.

112. The method of any one of claims 85-96 and 100-111, wherein the bacterial strain comprises Eubacterium biforme DSM 3989.

113. The method of any one of claims 85-96 and 100-112, wherein the bacterial strain comprises Eubacterium eligens DSM 3376.

114. The method of any one of claims 85-96 and 100-113, wherein the bacterial strain comprises Fusobacterium.prausnitzii ATCC 27768.

115. The method of any one of claims 85-96 and 100-114, wherein the bacterial strain comprises Howardella ureilytica GPC 589.

116. The method of any one of claims 85-96 and 100-115, wherein the bacterial strain comprises Megasphaera elsdenii LC1.

117. The method of any one of claims 85-96 and 100-116, wherein the bacterial strain comprises Parabacteroides johnsonii M-165.

118. The method of any one of claims 85-96 and 100-117, wherein the bacterial strain comprises Phascolarctobacterium sp. YIT 12067.

119. The method of any one of claims 85-96 and 100-118, wherein the bacterial strain comprises Roseburia hominus A2-183.

120. The method of any one of claims 85-96 and 100-119, wherein the bacterial strain comprises Ruminococcus callidus VPI 57-31.

121. The method of any one of claims 85-96 and 100-120, wherein the bacterial strain is selected from the group consisting of: Roseburia hominus A2-183, Eubacterium eligens DSM 3376, and a combination thereof.

122. The method of any one of claims 85-96 and 100-121, wherein the bacterial strain improves intestinal barrier function of the subject.

123. The method of any one of claims 85-96 and 100-122, wherein strain Roseburia hominus A2-183 has a 16S RNA gene that is at least 95% identical to SEQ ID NO:99.

124. The method of any one of claims 85-96 and 100-123, wherein strain Roseburia hominus A2-183 has a 16S RNA gene that is at least 95% identical to SEQ ID NO:100.

125. The method of any one of claims 85-96 and 100-124, wherein strain Roseburia hominus A2-183 has a 16S RNA gene that is at least 95% identical to SEQ ID NO:101.

126. The method of any one of claims 85-96 and 100-125, wherein strain Roseburia hominus A2-183 has a 16S RNA gene that is at least 95% identical to SEQ ID NO:102.

127. The method of any one of claims 85-96 and 100-126, wherein strain Roseburia hominus A2-183 has a 16S RNA gene that is at least 95% identical to SEQ ID NO:103.

128. The method of any one of claims 85-96 and 100-127, wherein strain Roseburia hominus A2-183 has a 16S RNA gene that is at least 95% identical to SEQ ID NO:104.

129. The method of any one of claims 85-96 and 100-128, wherein strain Eubacterium eligens DSM 3376 has a 16S RNA gene that is at least 90% identical to SEQ ID NO:73.

130. The method of any one of claims 85-96 and 100-129, wherein strain Eubacterium eligens DSM 3376 has a 16S RNA gene that is at least 95% identical to SEQ ID NO:74.

131. The method of any one of claims 85-96 and 100-130, wherein strain Eubacterium eligens DSM 3376 has a 16S RNA gene that is at least 95% identical to SEQ ID NO:75.

132. The method of any one of claims 85-96 and 100-131, wherein strain Eubacterium eligens DSM 3376 has a 16S RNA gene that is at least 90% identical to SEQ ID NO:76.

133. The method of any one of claims 85-96 and 100-132, wherein strain Eubacterium eligens DSM 3376 has a 16S RNA gene that is at least 95% identical to SEQ ID NO:77.

134. The method of any one of claims 85-96 and 100-133, wherein strain Eubacterium eligens DSM 3376 has a 16S RNA gene that is at least 95% identical to SEQ ID NO:78.

135. The method of any one of claims 85-96 and 100-134, wherein strain Eubacterium eligens DSM 3376 has a 16S RNA gene that is at least 95% identical to SEQ ID NO:79.

136. The method of any one of claims 85-96 and 100-135, wherein the bacterial strain in the composition is viable.

137. The method of any one of claims 85-96 and 100-136, wherein the bacterial strain is lyophilized.

138. The method of any one of claims 85-96 and 100-137, wherein the composition further comprises one or more cryopreservants.

139. The method of any one of claims 85-96 and 100-138, wherein the effective amount of the bacterial strain comprises at least about 1×10³ colony forming units (CFU) of the bacterial strain.

140. The method of any one of claims 85-96 and 100-139, wherein the effective amount of the bacterial strain comprises about 1×10⁴ to about 1×10¹⁵ CFU of the bacterial strain.

141. The method of any one of claims 85-96 and 100-140, wherein the effective amount of the bacterial strain comprises about 1×10⁶ to about 1×10¹⁰ CFU of the bacterial strain.

142. The method of any one of claims 85-96 and 100-141, wherein the bacterial strain in the composition is non-viable.

143. The method of claim 142, wherein the non-viable bacterial strain is heat-killed, irradiated, or lysed.

144. The method of any one of claims 85-143, wherein the method comprises administering the composition to the subject once, twice, or three times per day.

145. The method of any one of claims 85-144, wherein the composition is formulated for oral administration.

146. The method of any one of claims 85-145, wherein the composition is formulated for rectal administration.

147. The method of any one of claims 85-146, wherein the composition is formulated as a tablet, a capsule, a powder, or a liquid.

148. The method of any one of claims 85-147, wherein the composition is formulated as a tablet.

149. The method of claim 148, wherein the tablet is coated.

150. The method of claim 149, wherein the coating comprises an enteric coating.

151. The method of any one of claims 85-150, wherein the method further comprises administering another treatment for ulcerative colitis and/or adjunct therapy to the subject.

152. The method of claim 151, wherein the composition comprising the bacterial strain and the treatment for ulcerative colitis and/or adjunct therapy are administered simultaneously.

153. The method of claim 152, wherein the composition comprising the bacterial strain and the treatment for ulcerative colitis and/or adjunct therapy are administered sequentially.

154. The method of any one of claims 151-153, wherein the treatment for ulcerative colitis and/or adjunct therapy comprises a probiotic.

155. The method of any one of claims 151-154, wherein the treatment for ulcerative colitis and/or adjunct therapy comprises a therapeutic agent.

156. The method of claim 155, wherein the therapeutic agent comprises an anti-inflammatory agent, an immunosuppressant, an antibiotic, an anti-diarrheal agent, a pain reliever, an iron supplement, or a combination thereof.

157. The method of claim 156, wherein the anti-inflammatory agent comprises sulfasalazine, mesalamine, balsalazide, olsalazine, prednisone, hydrocortisone, rednisone, budesonide or a combination thereof.

158. The method of claim 155 or 156, wherein the immunosuppressant comprises azathioprine, mercaptopurine, cyclosporine, infliximab, adalimumab, golimumab, vedolizumab, or a combination thereof.

159. The method of any one of claims 155-158, wherein the composition comprising the bacterial strain further comprises the therapeutic agent.

160. The method of any one of claims 85-159, wherein the subject is a human.
